(19) United States
(12) Patent Application Publication
(10) Pub. No.: US 2024/0426799 A1
(43) Pub. Date: Dec. 26, 2024

(54) ENVIRONMENTAL-CONDITIONS SENSING APPARATUS FOR MODELING INTERNAL CONDITIONS OF ITEMS

(71) Applicant: LINEAGE LOGISTICS, LLC, Irvine, CA (US)

(72) Inventors: Nadia BOLIS, Berkeley, CA (US); Cameron SAYLOR, Davis, CA (US); Sam BAIJENS, Boxtel (NL); Kenneth NGUYEN, San Diego, CA (US); Sebastian PODIONO, San Diego, CA (US)

(21) Appl. No.: 18/738,659

(22) Filed: Jun. 10, 2024

Related U.S. Application Data
(60) Provisional application No. 63/509,686, filed on Jun. 22, 2023.

(51) Int. Cl. G01N 33/02

(52) U.S. Cl. CPC G01N 33/02

(57) ABSTRACT

Disclosed are systems and methods for detecting phase state information of objects in a facility. An apparatus can include a case with sensors. The sensors can detect environmental conditions related to an object and generate signals based on the detected conditions. The sensors can include temperature sensors, acoustic sensors, ultrasonic transducers, and/or humidity sensors. The apparatus can include a wireless transceiver to transmit the signals to a computer system. The computer system can process the signals using machine learning models to determine temperature and/or phase state information of the object, such as food products that are retained in/by the object. The apparatus can be removably affixed to the object. Sometimes, the apparatus can be positioned amongst many objects, such as multiple item cases on a pallet to determine an overall temperature and/or phase of the items on the pallet. The temperature/phase information can be used to optimize facility cooling operations.

### INCORPORATION BY REFERENCE

This application claims the benefit of priority of U.S. Provisional Patent Application No. 63/509,686, filed Jun. 22, 2023, the entirety of which is incorporated herein by reference.

### TECHNICAL FIELD

This document generally describes devices, systems, apparatuses, techniques, and methods related to modeling internal item conditions, such as temperature and/or phases of food products, in a controlled environment, such as during a cooling cycle in a facility (e.g., a cold storage warehouse), based on sensing and processing ambient conditions in the controlled environment.

### BACKGROUND

A facility, such as a storage facility, distribution center, and/or warehouse, can include one or more environment-controlled locations. The environment-controlled locations can include controlled environments, such as blast cells, and/or cold storage locations. The controlled environments, or blast cells, can be configured to cool or freeze items entering the facility to desired cooled conditions associated with the items before the items are then routed to storage locations in the facility, such as the cold storage locations. The cold storage locations can be temperature-controlled locations in the facility where items that require cold storage can be maintained until requested and delivered to customers or other relevant stakeholders.

The items can be contained in cases when entering the facility and when being stored. The cases can limit access to the items therein in order to protect and preserve conditions (e.g., freshness, food safety) of the items while in storage. The cases of items can also be loaded into the controlled environments for cooling or freezing the items inside the cases. The cases of items can remain inside the controlled environments for extended periods of time to ensure that the items appropriately cool to desired temperatures for the items while also maintaining food safety conditions. Because the items are contained in the cases during a cooling or freezing process, it can be challenging to know an exact, real-time temperature of the items to then determine when to remove the cases of items from the controlled environments. Sometimes, the cases of items can be removed prematurely, which can cause the items to spoil, perish, or otherwise not comply with food safety regulations. Sometimes, the cases of items can be removed significant amounts of time after the items have already reached desired temperatures, which can impact efficiency of operations in the facility as a whole.

The controlled environments can be blast cells. The controlled environments can include refrigeration systems or other types of cooling systems configured to cool or freeze the cases of items placed inside the controlled environments to desired temperatures for the items. Some facilities can have multiple controlled environments, which can be operated and controlled independently of each other. Some facilities can have fewer controlled environments.

### SUMMARY

The document generally describes technology, systems, devices, methods, techniques, and apparatuses for modeling temperature conditions and/or phase states of items (e.g., food products) in a facility, such as a storage facility, cold storage facility, and/or warehouse, by processing sensed ambient conditions surrounding the items. More particularly, the disclosed technology includes a sensor device that can be positioned adjacent to or on an outer surface of a case or pallet of items in the facility, such as in a blast cell or other cold storage/controlled location in the facility, to collect ambient conditions signals during predetermined time intervals. The signals can include, but are not limited to, temperature signals, acoustic signals, humidity signals, ultrasonic wave signals, and/or other types of sound signals. The collected signals can be transmitted to a computer system that can be configured to model the collected signals to determine internal conditions of the items inside the case/pallet. The internal conditions can include current temperature and/or humidity of the items, expected or projected temperature and/or humidity of the items, current phase state of the items, and/or expected or projected phase state of the items.

Currently, facilities with blast freezing or other cooling operations may not directly know when their items are finished freezing. One way to know is to measure the item's core temperature. However, this can be challenging to achieve since placing a temperature probe directly into the item can cause for food safety concerns, waste, and/or spoilage. As a result, to ensure that the items do not spoil from invasive temperature probe operations, many of the food items may be frozen or otherwise cooled for longer periods of time than necessary. The disclosed technology can therefore provide non-invasive techniques to accurately and efficiently detect when the items are frozen or have reached a desired internal temperature/phase state during blast freezing or other cooling operations to determine when to stop the blast freezing or cooling of the items. As described herein, temperature data and/or ultrasonic data can be collected and modeled using a variety of complex algorithms and/or trained machine learning models (e.g., neural networks (NNs), convolutional neural networks (CNNs), physics-based models, physics-informed machine learning) to make such determinations. Ultrasonic pulses and information, such as an acoustic impedance of water being different than ice, can additionally or alternatively be modeled to detect when the items finish phase transitioning to a solid and/or are ready to be removed from the blast freezing or other cooling operations.

The sensor device described herein can be configured to detect temperature, sound, humidity, ultrasonic waves, and/or other ambient conditions surrounding the case or pallet of items. For example, the sensor device can measure, in real-time or near real-time, supply air temperatures in the controlled environment having the case of items. The detected conditions can be transmitted to a computer system that can be configured to model the temperature conditions of the items inside the case using the detected conditions. The control system can provide the detected conditions as inputs to a machine learning model that was trained using training data that correlates different ambient conditions with internal item temperature readings. As another illustrative example, the sensor data can measure, at predetermined time intervals, ambient conditions, ultrasonic waves, and/or temperature signals, which can then be transmitted to the computer system and provided as inputs to one or more trained machine learning models. The machine learning models can be trained to process the inputs to determine likelihood that the items in the case or pallet are in a solid phase, a liquid phase, or a mixed phase (e.g., solid and liquid). The computer system may additionally process the model(s) determinations to identify a current, expected, and/or projected phase of the items.

Determinations made by the computer system (e.g., modeled temperature conditions of the items, phase of the items) can also be used by a control system or other computing systems associated with the facility to determine one or more operations to perform in the facility. As an illustrative example, the temperature conditions and/or the phase of the items can be used to determine when and for how long to cool or freeze the items in a controlled environment, such as a blast cell. The temperature conditions and/or the phase of the items can be used to determine when to remove the items from the controlled environment during a cooling cycle. The temperature conditions and/or the phase of the items can be used to determine one or more other controls of cooling cycles and/or operations of the controlled environment(s).

One or more embodiments described herein can include an apparatus for detecting phase state conditions of an object in a facility, the apparatus including: a case, a circuit board housed within the case, a group of sensors communicably connected to the circuit board, the group of sensors being configured to detect environmental conditions related to an object and generate signals based on the detected environmental conditions, the group of sensors including two or more of temperature sensors, ultrasonic transducers, acoustic sensors, or humidity sensors, a wireless transceiver inside the case and connected to the circuit board, the wireless transceiver being configured to transmit the signals from the group of sensors to a computer system, and a power source inside the case and configured to provide power to the group of sensors, the wireless transceiver, and the circuit board.

The apparatus can optionally include one or more of the following features. For example, the apparatus may also include a group of wires connected to the circuit board and at least partially extending from the case, the group of sensors being attached to endpoints of the group of wires and configured to extend to different portions of the object so that the group of sensors can detect the environmental conditions around the different portions of the object. The case can be removably affixed to a side of the object. The apparatus can be positioned amongst a group of objects on a pallet in a controlled environment, the controlled environment including at least one of a cold storage room, a blast cell, or a temperature-controlled transit vehicle.

One or more embodiments described herein can include a system for detecting phase state conditions of an object in a facility, the system including: a sensor device having a group of sensors that can be configured to generate signals indicating detected conditions associated with an object, the sensor device being configured to be removably affixed to the object, and a computer system in network communication with the sensor device, the computer system being configured to: receive the signals generated by the group of sensors, provide the received signals as inputs to one or more machine learning models, receive, as output from the machine learning models, information identifying potential conditions of the object, determining, based on applying one or more phase determination criteria to the information identifying the potential conditions of the object, a phase state of the object, and returning information about the phase state of the object.

The system can optionally include one or more of the following features. For example, the machine learning models could have been trained to generate values indicating likelihoods that the object is at one or more predetermined phases, the one or more predetermined phases including a solid phase, a liquid phase, and a mixed phase. The object can include a case of items, the items including food products. The group of sensors can include two or more of temperature sensors, acoustic sensors, ultrasonic transducers, or humidity sensors. The group of sensors can include acoustic sensors, the acoustic sensors including at least one transducer that can be configured to emit and receive an acoustic signal. Sometimes, the group of sensors can include a first transducer that can be configured to emit a sensor signal and a second transducer that can be configured to receive an echo or reflection of the emitted sensor signal.

Sometimes, determining, based on applying one or more phase determination criteria to the information identifying the potential conditions of the object, a phase state of the object can include: identifying a time interval to compute a moving average of the received signals, for each data point in the received signals for the identified time interval, computing an average of data points within the time interval centered at the data point, comparing the received signals to the computed moving averages, determining whether a threshold quantity of consecutive signals amongst the received signals satisfy the one or more phase determination criteria based on performing the comparison, and identifying the object as having a phase that corresponds to a phase of the threshold quantity of the consecutive signals amongst the received signals that satisfy the one or more phase determination criteria.

In some implementations, the one or more machine learning models could have been trained, by the computer system, in a process that may include: receiving ultrasonic wave signals and temperature signals for a group of objects, encoding or labeling the temperature signals with corresponding phase information, correlating the ultrasonic waves signals with the encoded temperature signals to generate correlated training data, and training the machine learning models to determine likelihoods of phases based on the correlated training data. The sensor device can be configured to be positioned amongst a group of objects on a pallet in the facility.

One or more embodiments described herein can optionally include a method for detecting phase state conditions of an object in a facility, the method including: receiving, from at least one sensor, environmental conditions detected in or around an object in a controlled environment, retrieving, from a data store, a model that may have been trained with machine learning techniques to correlate the detected environmental conditions with internal conditions of the object, applying the model to the detected environmental conditions, determining, based on applying the model, the internal conditions of the object, generating, based on applying one or more rules to the internal conditions of the object, cooling operations for the controlled environment, and returning the cooling operations for execution.

The method may optionally include one or more of the following features. For example, the object can include a case of items, and the model could have been trained using labeled or encoded training data to determine the internal conditions of a group of different types of items and a group of different packaging and materials of the case. The model may include parameters that can be adjustable based on a type of the items inside the case. The at least one sensor can be configured to (i) emit a wave signal that passes through the case and (ii) receive an echo or reflection of the wave signal when the wave signal reflects off a surface of the case that is opposite a location of the at least one sensor. The wave signal can include at least one of a soundwave signal or an ultrasonic wave signal. Sometimes, the method may also include: receiving the echo or reflection of the wave signal, filtering the echo or reflection of the wave signal with a band-pass filter, processing the filtered wave signal to determine a time of flight (TOF) for each reflection of the wave signal, and determining the internal conditions of the items inside the case based on applying the model to the TOF for each reflection of the wave signal.

In some implementations, determining, based on applying the model, the internal conditions of the object can include determining a percentage of the object that may be frozen. The model could have been trained using a process that may include: receiving temperature data for items inside a group of cases of items, the temperature data including ambient temperature conditions surrounding the group of cases of items and internal temperature conditions of the items inside the group of cases, applying a physics model to the received temperature data to correlate the ambient temperature conditions with the internal temperature conditions of the items inside the group of cases, and training the model to predict the internal temperature conditions of the items based on the correlated temperature data. Generating the cooling operation may include: generating a recommended action to complete a cooling cycle for the object, based on a determination that the internal conditions may satisfy one or more cooling criteria that correspond to the object.

One or more embodiments described herein can include an apparatus for detecting environmental conditions, the apparatus including: a case, a circuit board housed within the case, a group of wires connected to the circuit board and at least partially extending from the case, a group of sensors attached to endpoints of the group of wires, the group of sensors being configured to detect environmental conditions related to an object and generate signals based on the detected environmental conditions, the group of sensors including at least one of temperature sensors or acoustic sensors, a wireless transceiver inside the case that can be configured to transmit the signals from the group of sensors to a remote computing system in real-time or near real-time, and a power source inside the case that can be configured to provide power to the group of sensors and the wireless transceiver. The case can be configured to be removably affixed to the object, and the group of wires can be configured to extend to different portions of the object so that the group of sensors can detect the environmental conditions in or around the different portions of the object.

In some implementations, the embodiments described herein can optionally include one or more of the following features. For example, the object can be in a temperature-controlled environment. The temperature-controlled environment can be at least one of a cold storage room or a freezer. The temperature-controlled environment can be a blast cell. The acoustic sensors can include a transducer that can be configured to emit and receive an acoustic signal. The acoustic signal can be an ultrasound wave. The acoustic sensors can include a first transducer that can be configured to emit and acoustic signal and a second transducer that can be configured to receive an echo or reflection of the emitted acoustic signal. The object can be a case of items. The items can include food products. The object can be an item. The object can be a pallet of items. The environmental conditions can include ambient conditions in or around the object. The environmental conditions can include temperature conditions in or around the object. The environmental conditions can include audible conditions in or around the object.

As another example, the apparatus can also include a controller for the circuit board, the controller being configured to receive, process, and direct transmission of the signals from the group of sensors. The apparatus can include memory in the circuit board, the memory being configured to store the signals from the group of sensors over a predetermined period of time. The controller can also be configured to (i) combine one or more of the stored signals over the predetermined period of time and (ii) transmit, to the remote computing system via the wireless transceiver, one or more of the combined signals at an end of the predetermined period of time. The (i) combining and (ii) transmitting operations can be performed iteratively. The (i) combining and (ii) transmitting operations can be performed every time period. The wireless transceiver can be part of the circuit board. The wireless transceiver can be communicatively connected to the circuit board. The power source can be configured to provide power to the circuit board.

One or more embodiments described herein can include a method for detecting environmental conditions, the method including: receiving, by a control system and from at least one sensor, environmental conditions detected in or around an object, retrieving, by the control system and from a data store, at least one model that was trained with machine learning techniques to correlate the detected environmental conditions with internal conditions of the object, applying, by the control system, the at least one model to the detected environmental conditions, determining, by the control system, and based on applying the at least one model, the internal conditions of the object, generating, by the control system and based on the internal conditions of the object, cooling operation recommendations, and returning, by the control system, the cooling operation recommendations.

The method can optionally include one or more of the following features. For example, the object can include a case of items, and the at least one model can be trained to determine the internal conditions of a group of different types of items, the at least one model including parameters that can be adjustable based on a type of item inside the case. The parameters can include item type and packaging material of the case. The at least one sensor can be configured to emit a soundwave that can be configured to pass through the case and receive an echo of the soundwave when the soundwave reflects off a surface of the case that is opposite a location of the at least one sensor. The method can also include receiving, by the control system, the echo of the soundwave, filtering, by the control system, the echo of the soundwave with a band-pass filter, processing, by the control system, the filtered soundwave to determine a time of flight (TOF) for each reflection of the soundwave, and determining, by the control system, the internal conditions of the items inside the case based on applying the at least one model to the TOF for each reflection of the soundwave.

As another example, the internal conditions can include at least one of an object temperature, a current phase of the object, a percentage of the object that is frozen, or a percentage of the object that is unfrozen. Determining, by the control system, and based on applying the at least one model, the internal conditions of the object can include determining a current temperature of the object. Determining, by the control system, and based on applying the at least one model, the internal conditions of the object can include determining a current phase of the object, the current phase being at least one of a liquid phase, a solid phase, or a phase change. Determining, by the control system, and based on applying the at least one model, the internal conditions of the object can include determining a percentage of the object that is frozen.

In some implementations, the at least one model can be trained, by the control system, in a process including: receiving temperature data for items inside a group of cases of items, the temperature data including ambient temperature conditions surrounding the group of cases of items and internal temperature conditions of the items inside the group of cases, applying a physics model to the received temperature data to correlate the ambient temperature conditions with the internal temperature conditions of the items inside the group of cases, training the at least one model to predict the internal temperature conditions of the items based on the correlated temperature data, and returning the at least one model for runtime use.

Generating, by the control system and based on the internal conditions of the object, cooling operation recommendations can include: determining whether the internal conditions satisfy one or more cooling criteria that correspond to a type of the object. Generating, by the control system and based on the internal conditions of the object, cooling operation recommendations further can include: generating at least one recommended action for completing the cooling cycle for the object, based on a determination that the internal conditions satisfy the one or more cooling criteria that correspond to the object, and returning the at least one recommended action. The at least one recommended action can include instructions for removing the object from a temperature-controlled environment. The at least one recommended action can include instructions for stopping the cooling cycle for the object. The at least one recommended action can include instructions for routing the object to one or more storage locations within a storage facility.

Sometimes, the one or more cooling criteria can include threshold temperature values for a particular type of the object or food safety regulations that correspond to the object. Generating, by the control system and based on the internal conditions of the object, cooling operation recommendations further can include: generating at least one recommended action for adjusting the cooling cycle for the object, based on a determination that the internal conditions do not satisfy the one or more cooling criteria that correspond to the object, and returning the at least one recommended action. The at least one recommended action can include instructions for continuing the cooling cycle for the object until the internal conditions satisfy the one or more cooling criteria. The at least one recommended action can include instructions for at least one of (i) increasing a fan speed during the cooling cycle for the object for a predetermined amount of time or (ii) decreasing a temperature of a refrigeration system that is configured to provide supply air to cool the object. The at least one sensor can be removably affixed to an outer surface of the object and can be configured to detect the environmental conditions surrounding the object.

In some implementations, the at least one sensor can be a temperature sensor. The at least one sensor can be an acoustic sensor. The acoustic sensor can include a first transducer that can be configured to emit an acoustic signal and a second transducer that can be configured to receive an echo or reflection of the emitted acoustic signal. The first and second transducers can be positioned adjacent to each other on an outer surface of a shortest wall of the object. The object can be in a temperature-controlled environment that may contain a group of cases of items to be cooled during the cooling cycle inside the temperature-controlled environment. The temperature-controlled environment can be a freezer. The temperature-controlled environment can be a cold storage room. The temperature-controlled environment can be a blast cell. The object can be a case of items. The internal conditions of the object can include temperature conditions of the items inside the case. The internal conditions of the object can include temperature conditions of the items inside the case. The internal conditions of the object can include a phase of the items inside the case.

As another example, the at least one sensor can be a temperature sensor attached to a sensor housing, the sensor housing being positioned on an outer surface of the object. The outer surface can be a top outer surface of the object. The environmental conditions can include temperature signals that can be detected by the temperature sensor. A wire can be configured to extend from the sensor housing and the temperature sensor can be attached to an endpoint of the wire, the wire and the temperature sensor being configured to extend out from the sensor housing and around the object.

In some implementations, the at least one sensor can be a transducer that can be positioned on an outer surface of the object. The outer surface of the object can be a shortest side of the object. The environmental conditions can include soundwave signals that can be emitted and detected by the transducer. The method can also include retrieving, by the control system, current state data or a duration of a cooling cycle for the object. The method may include applying, by the control system, the model to the detected environmental conditions and the current state data or the duration of the cooling cycle for the object to determine the internal conditions of the object.

One or more embodiments described herein can include a system for detecting environmental conditions, the system including: a cooling system that can be configured to provide supply air to cool an object during a cooling cycle for the object, and a sensor device removably affixed to the object, the sensor device being configured to detect environmental conditions in or around the object in real-time or near real-time. The sensor device can include: at least one sensor that can be configured to detect the environmental conditions and generate signals based on the detected environmental conditions, a wireless transceiver that can be configured to transmit the signals to a control system in real-time or near real-time, a power source that can be configured to provide power to the at least one sensor and the wireless transceiver. The system can also include a control system in wireless communication with the cooling system and the sensor device and configured to perform operations that may include: receiving, via the wireless transceiver of the sensor device, the signals generated by the at least one sensor, retrieving, from a data store, at least one model that was trained to correlate the signals generated by the at least one sensor with internal conditions of the object during the cooling cycle, applying the at least one model to the received signals, determining, based on applying the at least one model, the internal conditions of the object, and returning the internal conditions of the object.

The system can optionally include one or more of the following features. For example, the system further can include at least one environmental sensor positioned near the object and separate from the sensor device. The at least one environmental sensor can be a temperature sensor that can be positioned near a vent through which the cooling system provides the supply air to the object. The at least one sensor can be a temperature sensor. The at least one sensor can be an acoustic sensor. The acoustic sensor can include a first transducer that can be configured to emit and acoustic signal and a second transducer that can be configured to receive an echo or reflection of the emitted acoustic signal.

The sensor device further can include a housing. The housing can be configured to enclose the wireless transceiver and the power source from the environmental conditions in the temperature-controlled environment, the at least one sensor being configured to extend from the housing. The sensor device further can include at least one adhesive attached to a bottom surface of the housing that can be configured to removably affix the sensor device to an outer surface of the object.

In some implementations, the internal conditions can include at least one of an object temperature, a current phase of the object, a percentage that the object is frozen, or a percentage that the object is unfrozen. Generating, based on the internal conditions of the object, cooling operation recommendations can include: determining whether the internal conditions satisfy one or more cooling criteria that correspond to the object, generating instructions to perform at least one action for completing the cooling cycle for the object, based on a determination that the internal conditions satisfy the one or more cooling criteria that correspond to the object, and transmitting the instructions to a controller of the cooling system. The controller of the cooling system can be configured to execute the instructions to cause the cooling system to complete the cooling cycle for the object. The instructions can include at least one of: (i) stopping or pausing the cooling cycle for a predetermined period of time, (ii) routing the object to one or more storage locations within a storage facility, or (iii) grouping the object with other objects having similar internal conditions. The system may also include a temperature-controlled environment that can be configured to hold a group of objects to be cooled to respective freezing temperatures during the cooling cycle. The temperature-controlled environment can be at least one of a freezer, a cold storage room, or a blast cell.

One or more embodiments described herein can include an apparatus for detecting environmental conditions, the apparatus including: a case, a circuit board housed within the case, a group of sensors communicably connected to the circuit board, the group of sensors being configured to detect environmental conditions related to an object and generate signals based on the detected environmental conditions, a wireless transceiver inside the case and connected to the circuit board, the wireless transceiver being configured to transmit the signals from the group of sensors to a remote computing system in real-time or near real-time, and a power source inside the case that can be configured to provide power to the group of sensors, the wireless transceiver, and the circuit board.

The apparatus can optionally include one or more of the following features. For example, the apparatus can also include a group of wires connected to the circuit board and at least partially extending from the case, the group of sensors being attached to endpoints of the group of wires. The group of wires can be configured to extend to different portions of the object so that the group of sensors detect the environmental conditions in or around the different portions of the object. The group of sensors can include at least one of temperature sensors or acoustic sensors. The case can be configured to be removably affixed to the object.

The devices, system, and techniques described herein may provide one or more of the following advantages. For example, the disclosed technology provides accurate, efficient, real-time or near real-time detection of temperature conditions and/or phase states of items using minimal inputs. Traditionally, a facility can include temperature sensors positioned throughout the facility. Although the temperature sensors may track ambient temperature conditions, the temperature sensors may not provide accurate readings of temperature conditions of items in the facility. The disclosed technology leverages conditions detected by a sensor device surrounding the items to accurately model real-time or near real-time internal temperature and/or phase conditions of the items. The disclosed technology is able to non-invasively and accurately determine the internal temperature conditions and/or phases of the items, which can then be used to modify operations of facility components (e.g., modify a cooling cycle in a controlled environment housing the items) on the fly, in real-time, or near real-time. The modeling techniques described herein can provide improved accuracy in item temperature and/or phase detection without actually probing or causing damage to the items. Moreover, because minimal inputs are being used to model the temperature conditions, the disclosed technology may use less processing power and compute resources (e.g., less RAM, less memory), which speeds up processing efficiency and allows for accurate real-time or near real-time modeling of temperature conditions and control of the facility components.

The disclosed techniques also can provide for efficient use of the facility and scheduling of facility operations by recommending to remove the cases of items from the controlled environment as soon as the items reach desired temperature and/or phase conditions. As a result, the controlled environment can be emptied then filled with another load of items to be cooled so that the controlled environment is efficiently being used rather than sitting idle for extended periods of time. Cooling cycles and other operations in the facility can be performed more efficiently and optimally. In addition, the disclosed technology can cause energy efficiencies and savings based on real-time or near real-time control and adjustments of the facility components.

As another example, the disclosed techniques can be less expensive to implement in different types of facilities. The disclosed technology, such as the sensor device, can be built with off-the-shelf components, such as sensors and wireless receivers. The sensor device can be retrofitted to any existing facility by simply placing the sensor device inside controlled environments (e.g., on shelves, attached to walls, attached to a floor, etc.) and/or attaching/affixing the sensor device directly to one or more cases of items. The sensor device can be easily and quickly removed or detached from the cases of items and attached/affixed to other cases of items. As a result, the disclosed technology can be easily used and implemented in various settings and/or use cases in the facility without requiring expensive, extensive, or complex installations and integrations with existing technology and components in the facilities.

The disclosed techniques can also provide improved data collection and data processing for optimizing various facility operations. The disclosed techniques can be expanded to model and generate optimized scheduling operations for controlled environments. The disclosed techniques can be expanded to model and generate optimized routing operations for the cases of items to be cooled and/or after the cases of items are cooled. As another example, the disclosed techniques can be used in combination with one or more other inputs for generating simulations of facility operations. The disclosed techniques can also be used to model and fine-tune control of physical features of the controlled environments, including but not limited to operational conditions of fans, cooling units, refrigeration systems, and/or other aspects of the controlled environments, such as quantity, placement, and/or layout of racks inside the controlled environments that can receive the cases of items to be cooled therein.

As yet another example, the sensor device described herein can be designed to protect components, such as a power source, sensors, and a wireless transceiver so that detected conditions (e.g., sensor signals) can be transmitted in real-time or near real-time to a computing system for further processing. As a result, components of the sensor device can continue to operate as expected under any environmental conditions, regardless of how cold, humid, etc. the environmental conditions may be.

As another example, the disclosed techniques can provide for internal temperature values and/or phase states of food items to be non-invasively modeled, predicted, or otherwise determined using complex algorithms and machine learning models, and without causing damage to the items. As a result, the items may not be damaged or spoiled while in transit and/or storage, up until the items are provided to end consumers. Not only can this improve and/or maintain end consumer expectations about the items they are purchasing and/or receiving, the disclosed techniques can also provide for reducing or otherwise avoiding liability of the facility and/or other middlemen between item providers and end consumers.

To provide robust and accurate temperature/phase detection of items in the facility, the disclosed technology can use a complex collection of algorithms and/or machine learning techniques to analyze data related to at least one parameter (e.g., temperature, humidity, sound, ultrasonic waves) for a case or pallet of items to inform relevant users associated with the facility about current, expected, and/or projected temperature and/or phase states of the items. Exemplary and complex techniques described herein can include: taking inputs from multiple sensors, selecting some data provided by the sensors, ignoring some of the data that was provided by the sensors, performing multiple calculations on a selected subset of the data, combining the data from these multiple calculations and then outputting that data within a short amount of time, all for multiple relevant users. This complex collection of algorithms and/or machine learning techniques can provide an unconventional solution to the problem of trying to detect temperature and/or phase information of items in the facility, such as during cooling operations. Traditional systems of detecting ambient temperature conditions in the facility may not provide the disclosed solutions for at least the following reasons: (i) the significant processing power required for to continuously monitor conditions of a particular case of items and detect temperature and/or phase states of the case of items, (ii) the considerable data storage requirements for maintaining information collected and determined by the disclosed technology, (iii) a large enough pool of parameter data to provide accurate thresholds for the disclosed algorithms and/or machine learning techniques, and/or (iv) algorithms and/or machine learning techniques that allow for the thresholds to be self-updated in light of additional data that can be added to the pool of relevant parameter data. The disclosed technology may require analyzing many data points to find similarities amongst ambient conditions, determining parameters associated with different temperature conditions and/or phase states, determining how these parameters change over time to accurately identify a temperature and/or phase of items in the facility, generating and outputting information to the relevant users based on the parameters, the identified/determined temperature of the items, the identified/determined phase of the items, and then repeating the above operations over a relatively short time period (e.g., every day, every half day, every hour, every 10 minutes, every 5 minutes, every 1 minute) and for many different cases/pallets of items and/or many different facilities.

The details of one or more implementations are set forth in the accompanying drawings and the description below. Other features and advantages will be apparent from the description and drawings, and from the claims.

### BRIEF DESCRIPTION OF THE DRAWINGS

FIG. 1A is a conceptual diagram of a system for adjusting a controlled environment based on modeling temperature conditions of items inside the controlled environment.

FIG. 1B is a conceptual diagram of a system for adjusting a controlled environment based on modeling temperature conditions of items inside the controlled environment.

FIG. 2A illustrates an example sensor device for measuring conditions around and/or inside a case of items.

FIG. 2B illustrates another implementation of the example sensor device for measuring conditions around and/or inside a case of items.

FIG. 3 is a conceptual diagram of a system for training at least one model to predict item temperature conditions during a cooling cycle inside a controlled environment.

FIG. 4A illustrates an example case of items with sensors used for training at least one model to predict item temperature conditions during a cooling cycle.

FIG. 4B illustrates an example case of items with a sensor device used at runtime to predict item temperature conditions during a cooling cycle.

FIG. 4C illustrates an example case of items with a sensor device used at runtime to predict internal item conditions during a cooling cycle.

FIG. 5 is a conceptual diagram of a system for modeling item temperature conditions during a cooling cycle inside a controlled environment based on soundwaves.

FIG. 6 is a flowchart of a process for training at least one model to predict item temperature conditions during a cooling cycle inside a controlled environment based on soundwaves.

FIG. 7A illustrates example soundwaves measured inside a case of items during a cooling cycle that indicate different phases of the cooling cycle.

FIGS. 7B, 7C, and 7D illustrate example graphs for measuring speed of sound as it travels through cases of items using the disclosed techniques.

FIG. 7E illustrates an example subtraction method for determining a phase of items based on acoustic signals.

FIGS. 8A and 8B are a flowchart of a process for adjusting a controlled environment based on modeling item temperature conditions inside the controlled environment during a cooling cycle and using temperature data.

FIGS. 9A and 9B are a flowchart of a process for adjusting a controlled environment based on modeling item temperature conditions inside the controlled environment during a cooling cycle and using sound data.

FIGS. 10A and 10B illustrate example system components that can be used to perform the disclosed techniques.

FIG. 11 is a schematic diagram that shows an example of a computing device and a mobile computing device.

FIG. 12 illustrates an example pallet having a sensor device positioned between cases of items on the pallet.

FIG. 13A is a conceptual diagram of a system for determining a phase state of items in a facility based on modeling ultrasonic wave signals.

FIG. 13B is a conceptual diagram of a sensor device configured to attach to a case of items to generate and receive ultrasonic wave signals.

FIG. 14 illustrates the sensor device of FIG. 13B.

FIGS. 15A, 15B, and 15C are a flowchart of a process for determining a phase state of items based on modeling ultrasonic wave signals.

FIG. 16 is a flowchart of a process for training a phase model based on ultrasonic wave signals and temperature signals.

FIG. 17 is a flowchart of a process for determining a phase state of items using one or more trained machine learning models.

FIG. 18 is an example graph depicting a phase change detection using the disclosed technology.

Like reference symbols in the various drawings indicate like elements.

### DETAILED DESCRIPTION OF ILLUSTRATIVE EMBODIMENTS

This document generally relates to systems, methods, techniques, and apparatuses for detecting ambient conditions surrounding items in a facility to determine internal conditions of the items. For example, the disclosed technology may include processing and modeling sensor signals (e.g., temperature, humidity, acoustic, ultrasonic) collected proximate the items in the facility to determine current, expected, and/or projected temperature and/or phase states of the items. The disclosed technology can be used to measure temperatures and/or phase states of items in real-time or near real-time as the items are being cooled or frozen during a cooling cycle/cooling operations in a controlled environment, such as a blast cell. Using the disclosed technology, a computer system can determine appropriate times to stop the cooling cycle, remove the items from the controlled environment, load the controlled environment with other items, and/or perform other cooling cycles for the other items. As a result, the disclosed technology may allow for efficient and accurate, real-time or near real-time detection of ambient conditions to determine internal temperature conditions of the items and modify or otherwise optimize operations in the facility as a whole.

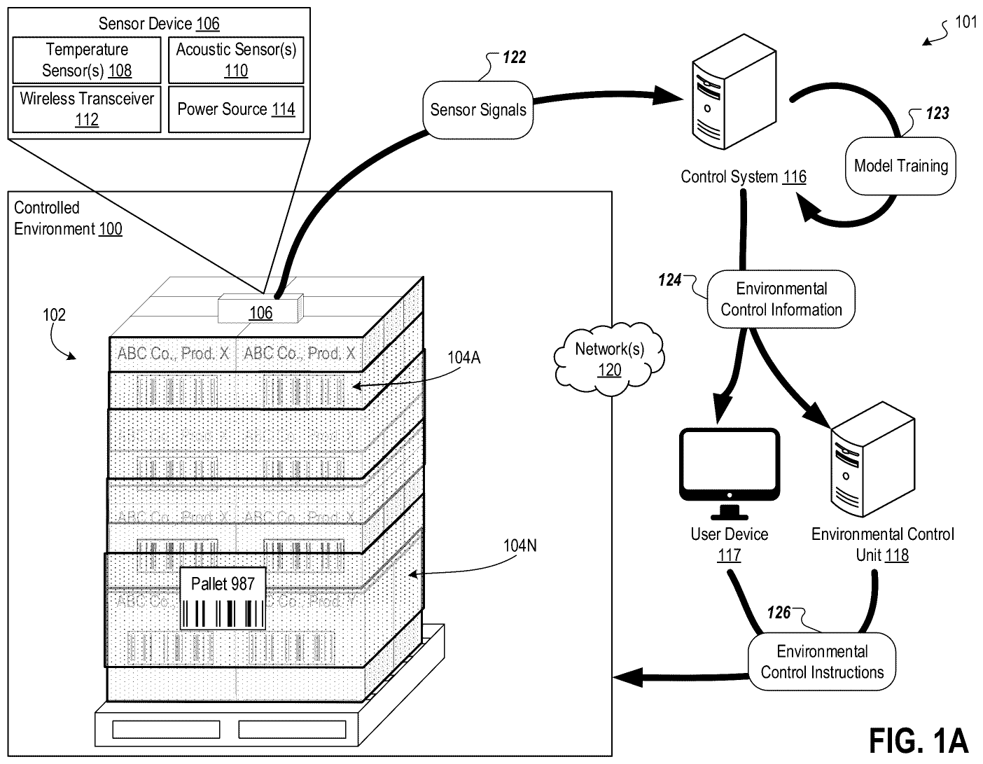

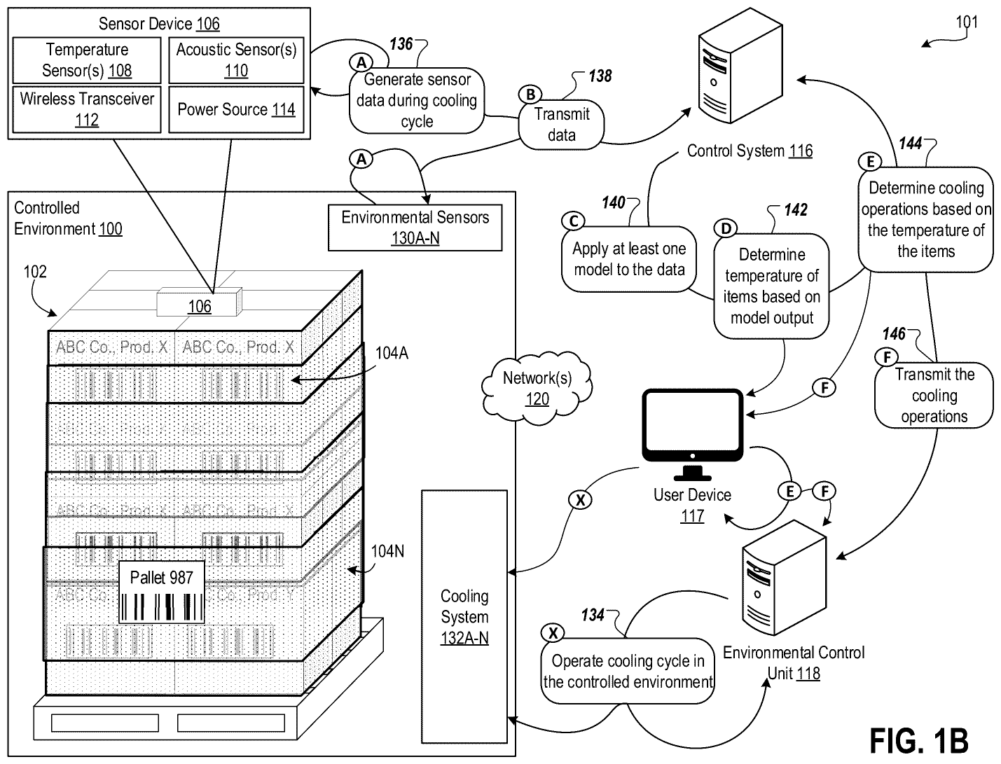

Referring to the figures, FIG. 1A is a conceptual diagram of a system 101 for adjusting a controlled environment 100 based on modeling temperature conditions of items inside the controlled environment 100. The system 101 can be a facility, such as a cold storage facility, storage facility, distribution center, and/or warehouse. The system 101 can include at least one controlled environment 100, a control system 116, a user device 117, and an environmental control unit 118 communicating with each other (e.g., wired, wirelessly) via network(s) 120. In some implementations, the control system 116 and/or the environmental control unit 118 may be separate/remote from the system 101.

The controlled environment 100 can be a blast cell or similar type of structure, room, and/or location in the system 101 (e.g., facility) that is configured to receive items and perform cooling cycles therein to cool or freeze the items. The controlled environment 100 can include refrigeration and/or cooling systems, described further below, to cause the items placed therein to be cooled to desired freezing temperatures for the respective items. Components for cooling the items inside the controlled environment 100 can be determined and/or controlled by the environmental control unit 118. In some implementations, each of the components in the controlled environment 100 can be individually controlled by respective controllers.

In the example of FIG. 1A, a pallet 102 having cases 104A-N of items can be placed inside the controlled environment 100 for a cooling cycle. The controlled environment 100 can receive multiple quantities of pallets having cases of items. In some implementations, the controlled environment 100 can include racks, layers, and/or levels to receive the cases 104A-N of items. Sometimes, the pallet 102 can have all items of a same type. Sometimes, the pallet 102 can have mixed types of items. In some implementations, the controlled environment 100 can receive pallets all having different types of items. The cases 104A-N can contain items requiring some level of cooling and/or freezing, including but not limited to perishable goods, food products, produce, vegetables, fruits, meats, fishes, food liquids, frozen foods, etc.

The control system 116 can be any type of computing system, computer, computing device, cloud-based computing system, or network of computing devices/systems. The control system 116 can be configured to determine, in real-time or near real-time, various conditions inside the controlled environment 100, such as temperature and sound, and model internal conditions of the items placed therein based on the determined conditions. The internal conditions of the items can include internal temperature values. The control system 116 can determine the various conditions inside the controlled environment 100 based on sensor signals that are detected by and received from one or more sensors positioned inside the controlled environment 100.

The user device 117 can be any type of computing system, device, mobile device, mobile phone, smart phone, tablet, laptop, and/or computer. The user device 117 can be configured to present information in one or more graphical user interfaces (GUIs) to relevant users, such as a facility manager, an operations manager, or any other user that may be adjusting and/or controlling components in the system 101.

The environmental control unit 118 can be any type of controller, computing system, computer, computing device, cloud-based computing system, or network of computing devices/systems. In some implementations, the environmental control unit 118 can be part of the control system 116. The environmental control unit 118 can be configured to determine one or more controls for the components of the controlled environment 100. For example, the environmental control unit 118 can determine whether to pause, start, or stop a cooling cycle inside the controlled environment 100. The environmental control unit 118 can determine whether one or more fan speeds can be increased or decreased to change an amount of time that it may take to cool the items inside the controlled environment 100 to their respective desired temperatures. The environmental control unit 118 can also determine one or more other controls, operations, and/or adjustments to existing controls for components such as cooling systems and/or refrigeration systems that impact the cooling cycle in the controlled environment 100.

A sensor device 106 can be removably affixed/attached to any portion of the cases 104A-N of items on the pallet 102. The sensor device 106, in the example of FIG. 1A, is affixed to a top portion of the cases 104A-N on the pallet 102. In some implementations, the sensor device 106 can be affixed along a length of any of the sides of the cases 104A-N on the pallet 102. In some implementations, the sensor device 106 can be affixed to one or more walls inside the controlled environment 100 and/or one or more components in the controlled environment 100 (e.g., on or near a supply air vent near a top surface or ceiling of the controlled environment, on or near a return air vent near a bottom surface or floor of the controlled environment).

The sensor device 106 can be configured to measure ambient conditions (e.g., temperature) in the controlled environment 100 near/proximate to the cases 104A-N of items. The measured ambient conditions can then be transmitted to the control system 116 for further processing, as described further below. Accordingly, the sensor device 106 can include at least one temperature sensor 108, at least one acoustic sensor 110, a wireless transceiver 112, and a power source 114. In some implementations, the sensor device 106 may only have one type of sensor (e.g., a temperature sensor or an acoustic sensor, such as a transducer). In some implementations, the sensor device 106 can have a plurality of group of sensors of varying sensor types.

In brief, the temperature sensor 108 can be configured to detect, in real-time or near real-time, temperature values surrounding or adjacent to the cases 104A-N of items on the pallet 102. The acoustic sensor 110 can be configured to transmit and receive/detect acoustic soundwaves or other types of soundwaves in an area surrounding, adjacent to, or otherwise inside the cases 104A-N in the controlled environment 100. As described herein, the acoustic sensor 110 can be configured to transmit and receive/detect the soundwaves passing through any of the items in the cases 104A-N in the controlled environment 100, as described further herein. The wireless transceiver 112 can be configured to transmit signals generated by the sensors 108 and 110 to the control system 116. The wireless transceiver 112 can also be configured to receive signals and/or instructions from the control system 112 or other computing systems in the system 101, the signals and/or instructions being executed at the sensor device 106 to cause the sensors 108 and/or 110 to measure conditions in the controlled environment 100, generate signals for the measured conditions, and transmit the signals to the control system 116 or other computing systems described herein. The power source 114 can include a battery, solar panel (e.g., solar panel provides power to the facility, which can power the sensor device 106), or other type of charging device that can provide power to the components of the sensor device 106.

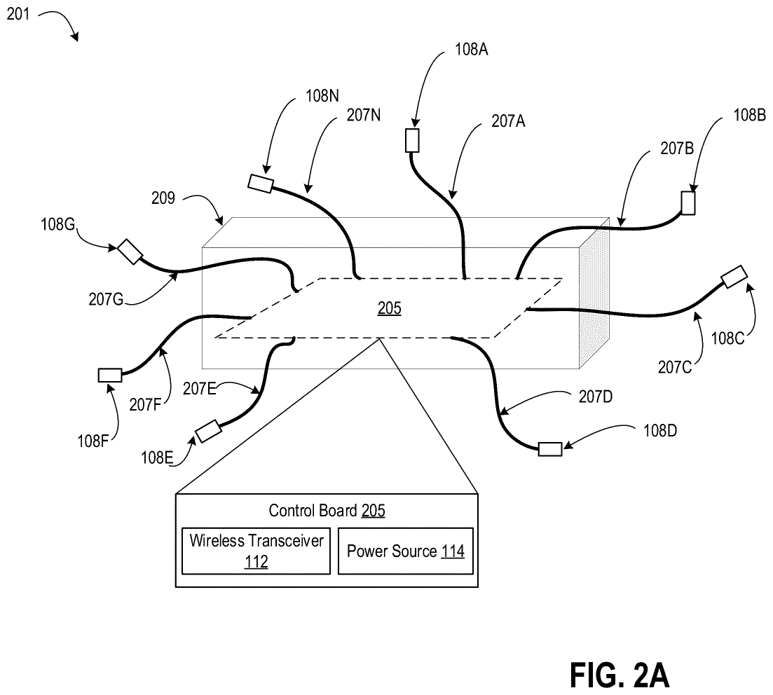

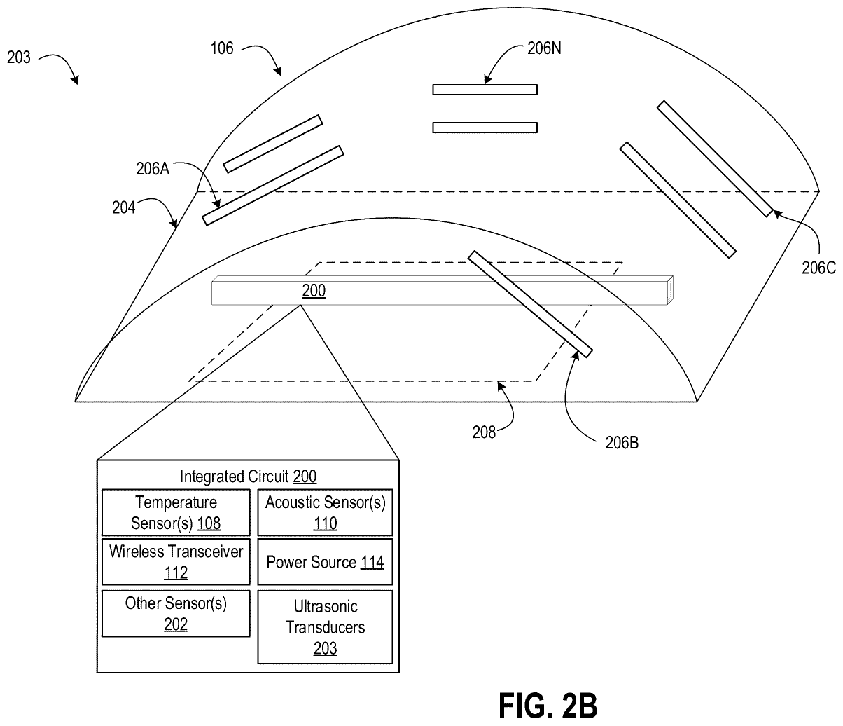

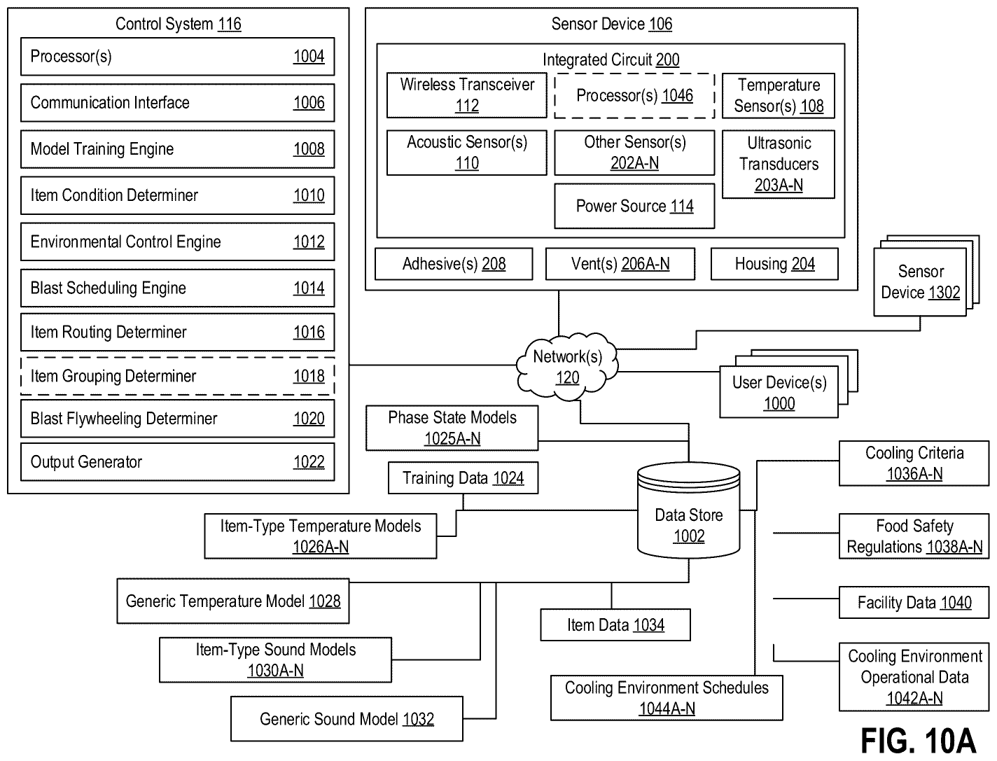

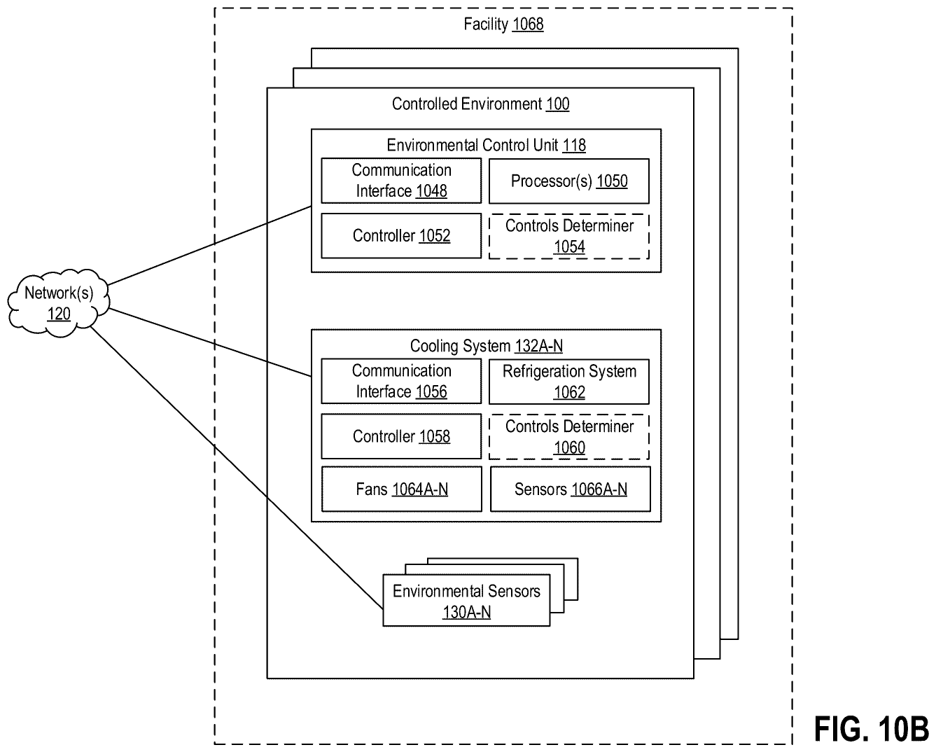

The sensor device 106, as described further in reference to FIGS. 2A-B, can include a housing configured to protect the components 108, 110, 112, and 114 from extreme ambient conditions, such as freezing temperatures, humidity, condensation, and other conditions that may cause any of the components to lag, slow down, or otherwise not properly detect and transmit information. As a result, the components 108, 110, 112, and 114 can continue to operate in real-time or near real-time to provide for up-to-date relay of information between components described herein. The components of the sensor device 106 can also be rated down to a predetermined temperature value (e.g., −40° C.) so that the components can operate as intended or expected regardless of surrounding ambient conditions/temperature. Refer to at least FIGS. 2A, 2B, 10A and 10B for further discussion about the components of the sensor device 106.

Still referring to the system 101 in FIG. 1A, the sensors 108 and/or 110 of the sensor device 106 can detect ambient conditions in the controlled environment 100 and provide sensor signals 122 of the detected conditions to the control system 116. The sensor signals 122 can include temperature values on a top surface of the cases 104A-N where the sensor device 106 is affixed. The sensor signals 122 can include soundwaves that are measured when soundwaves are emitted into the cases 104A-N and reflected or echoed back to the emitting sensor 110 or a receiving sensor 110. The sensor signals 122 can additionally or alternatively include ambient conditions that are detected surrounding the cases 104A-N of items on the pallet 102.

Using the sensor signals 122, the control system 116 can generate environmental control information 124. As described further below, the control system 116 can provide the sensor signals 122 as input to one or more machine learning models (e.g., physics model) that were trained to correlate the sensor signals 122 with internal temperature conditions of the items in the cases 104A-N. The control system 116 can determine the environmental control information 124 based on the internal temperature conditions of the items in the cases 104A-N, such as determining when to remove the pallet 102 from the controlled environment 100, when to pause, start, and/or stop a cooling cycle in the controlled environment 100, how to adjust one or more of the components used for performing the cooling cycle in the controlled environment 100, and/or other types of operations that can be adjusted/modified in the system 101 to improve efficiency therein. In some implementations, the environmental control information 124 can indicate internal conditions of the items in the cases 104A-N, including but not limited to a current temperature of the items in the cases 104A-N, a phase change or current phase of the items in the cases 104A-N, an amount of time that the items are in one or more phases, etc.

The control system 116 can transmit the environmental control information 124 to the user device 117 and/or the environmental control unit 118. The user device 117 can present the environmental control information 124 to the user. The user can use the information presented at the user device 117 to determine what operations or actions to perform with regards to the controlled environment 100.

The user device 117 and/or the environmental control unit 118 can use the environmental control information 124 to generate and/or update environmental control instructions 126. The user can provide input at the user device 117 indicating instructions to adjust operations of a cooling system for the controlled environment 100. The instructions can then be executed by the environmental control unit 118 and/or a controller of the cooling system. As another example, the environmental control unit 118 can generate the instructions. The environmental control instructions 126 can be executed by components that affect the cooling cycle in the controlled environment 100. In some implementations, the environmental control unit 118 can determine one or more controls for the components of the controlled environment 100 (such as starting, stopping, or pausing cooling cycles, adjusting fan speed(s) inside the controlled environment, turning one or more fans on/off, etc.). These controls can then be transmitted to the user device 117 and presented in the GUIs. The relevant user can view the controls and determine whether to implement any of the controls for the components of the controlled environment 100.

The control system 116 can also train the models described herein (block 123). The sensor signals 122, the environmental control information 124, and/or the environmental control instructions 126 can be provided as inputs in a training loop at the control system 116 to iteratively train and improve the model(s).

Although the system 101 is shown and described in FIG. 1A in relation to one controlled environment 100 (e.g., blast cell), the disclosed techniques can also be used for modeling temperature conditions of items in many different controlled environments inside a facility.

FIG. 1B is a conceptual diagram of the system 101 of FIG. 1A for adjusting the example controlled environment 100 based on modeling temperature conditions of items inside the controlled environment 100. Similar to the configuration of the system 101 in FIG. 1A, the system 101 in FIG. 1B includes the sensor device 106, the control system 116, the user device 117, and the environmental control unit 118 communicating (e.g., wired, wirelessly) via the network(s) 120. In some implementations, the user device 117 can be the same as the control system 116 and/or the environmental control unit 118. The system 101 can also include environmental sensors 130A-N and cooling systems 132A-N. The environmental sensors 130A-N and the cooling systems 132A-N can be positioned inside the controlled environment 100. In some implementations, one or more of the environmental sensors 130A-N and the cooling systems 132A-N can be positioned outside or adjacent to the controlled environment 100.

In brief, the environmental sensors 130A-N can include one or more different types of sensors configured to detect ambient conditions inside the controlled environment 100. The sensors 130A-N can include but are not limited to temperature sensors, humidity sensors, presence sensors, motion sensors, etc. The sensors 130A-N can be positioned inside the controlled environment 100, but unlike the sensors 108 and 110, the sensors 130A-N may not be attached/affixed to surfaces of the cases 104A-N of items. The environmental sensors 130A-N can be configured, for example, to measure supply air temperature as the supply air enters the controlled environment 100 according to instructions executed by the cooling systems 132A-N.

The cooling systems 132A-N can be any type of cooling or refrigeration system that is configured to lower a temperature inside the controlled environment 100 to cool or freeze the items placed therein. The cooling systems 132A-N can include fans, vanes, and other refrigeration components for circulating cold supply air into and through the controlled environment 100.

During runtime, the sensor device 106 can be positioned, attached, and/or affixed to an outer surface of one or more of the cases 104A-N. The temperature sensor(s) 108 can be a probe configured to measures ambient air temperature that is adjacent to the cases 104A-Non the pallet 102. In some implementations, multiple sensor devices can be used in the controlled environment 100. The sensor devices 106 can be attached to cases on various different pallets in different locations in the controlled environment 100. For example, a first sensor device can be attached to at least one pallet that is positioned in a worst location for blast freezing/cooling (e.g., a location where pallets of items cool to desired temperatures over longer periods of time because the location is farther away from vents, vanes, plenums, and/or fans that provide supply air into the controlled environment 100). The first sensor device can be attached to a pallet that is located on a lowest shelf and closest to a return air vent, which can be farthest away from an upper shelf/rack closest to a supply air vent. As another example, a second sensor device can be attached to at least one pallet that is positioned in a middle of the controlled environment 100 (e.g., a midpoint of the controlled environment, a middle shelf). As yet another example, a third sensor device can be attached to at least one pallet that is positioned in a best location for blast freezing/cooling (e.g., a location where pallets of items cool to desired temperatures over shorter periods of time because the location is closest to vents, vanes, plenums, and/or fans that provide supply air into the controlled environment 100. The third sensor device can be attached to a pallet that is located on a top shelf and closest to a supply air vent. In some configurations of the controlled environment 100, higher-up rows, racks, and/or shelves can be worst locations for blast freezing/cooling because such locations can be farthest away from the supply air. Sometimes, air circulation may not be uniform in the controlled environment 100, in part because the air cannot turn around fast enough near a top surface/ceiling of the controlled environment 100. In some configurations, sides of the controlled environment 100 can include the best locations for blast freezing/cooling in comparison to middle locations because the air can circulate and flow faster and more easily on the sides versus between and/or through cases of items in the middle of the controlled environment 100.

Still referring to FIG. 1B, the environmental control unit 118 can operate a cooling cycle in the controlled environment (block X, 134). The cooling cycle can be executed at predetermined times (e.g., based on instructions from the control system 116 and/or based on instructions associated with the cooling cycle). The cooling cycle can be executed whenever the controlled environment 100 is filled to a minimum capacity of pallets 102. Executing the cooling cycle can include controlling the cooling systems 132A-N to cool the cases 104A-N of items inside the controlled environment 100. Block X (134) can be performed before, during, and/or after any of the other blocks/operations described herein.

The temperature sensor(s) 108, the acoustic sensor(s) 110, and/or the environmental sensors 130A-N can generate sensor data during the cooling cycle inside the controlled environment 100 (block A, 136). The sensor data can include temperature values detected in ambient air surrounding the pallet 102 having the cases 104A-N of items or otherwise inside the cases 104A-N themselves. The sensor data can include soundwaves detected inside one or more of the cases 104A-N on the pallet 102. In some implementation, the sensor data can include soundwaves detected in the ambient air surrounding the pallet 102 and/or soundwaves detected as reflecting or echoing off of one or more of the cases 104A-N on the pallet 102. One or more other types of sensor data can be generated by the sensors described herein.

Any of the sensors 108, 110, and/or 130A-N can transmit the sensor data to the control system 116 (block B, 138). The sensor data can be transmitted in real-time, as ambient conditions are detected in the controlled environment 100. The sensor data can be transmitted in real-time since the components of the sensor device 106 can be protected in a housing that ensures proper functioning of the components in any type of inclement conditions (e.g., freezing temperatures, etc.). Sometimes, the sensors 108, 110, and/or 130A-N can be configured to transmit the sensor data in near real-time.

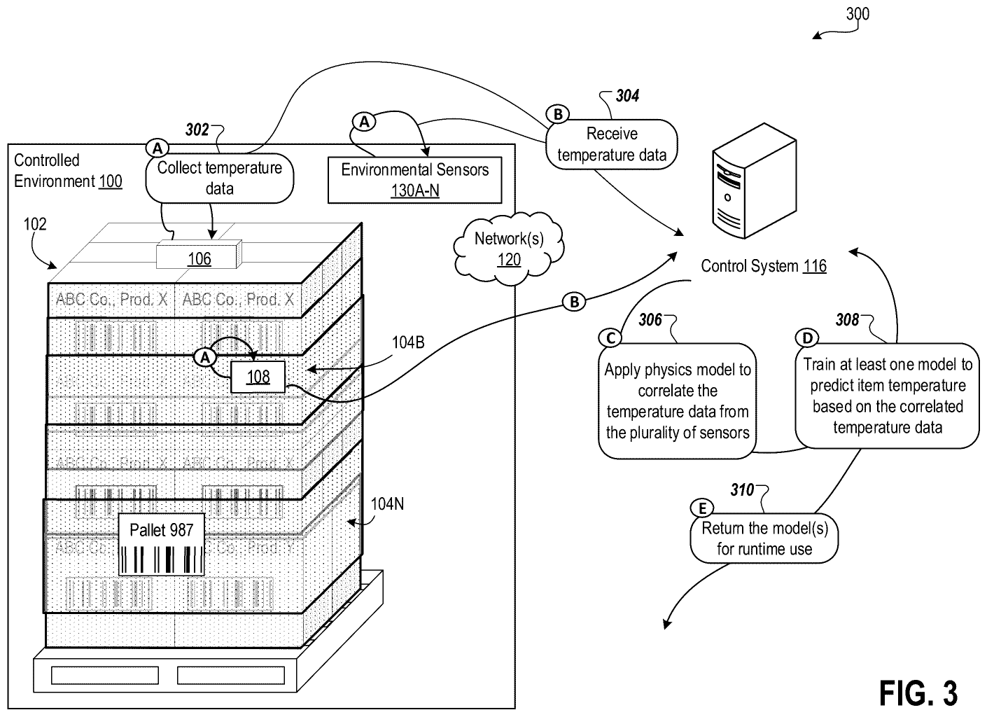

The control system 116 can apply at least one model to the received sensor data in block C (140). The model can receive, as inputs, temperature conditions around the cases 104A-N, soundwaves detected through the cases 140A-N, temperature conditions near supply air vents in the controlled environment 100, and/or information about the items in the cases 104A-N(e.g., item type, desired freezing temperature for the items). The model, as described further in reference to FIGS. 3, 4A, 4B, 5, and 6 can be trained to determine, based on the inputs, projected internal conditions of the items in the cases 104A-N. The model can be trained with physical modeling techniques for purposes of detecting internal temperature conditions for solid food items, such as meats, chicken, etc., and/or internal conditions (e.g., temperature, current phase, phase change) of liquid items, such as soups, concentrates, etc. The model can be trained with other modeling techniques to detect internal temperature conditions of liquid food items, such as soups, concentrates, etc.

Moreover, the model can be trained to take into consideration a variety of factors and/or inputs, including but not limited to a quantity of items or pallets of items in the controlled environment 100. For example, if a particular pallet of items is surrounded by other pallets of items, an ambient temperature value detected by the temperature sensor(s) 108 can be higher as a result of total temperature of the other pallets of items. Therefore, the total quantity of pallets in the controlled environment 100 can be used as a factor to determine that the particular pallet of items may need to be inside the controlled environment 100 for longer to reach a desired freezing temperature.

Accordingly, the control system 116 can determine a temperature of items inside the cases 104A-N based on the model output (block D, 142).

The computer system 116 can also determine one or more cooling operations based on the temperature of the items in block E (144). The cooling operations can indicate when the cases 104A-N of the items can or should be taken out of/removed from the controlled environment 100 (e.g., based on the determined temperature of the items reaching desired freezing temperature conditions for the particular type of items). The cooling operations can additionally or alternatively include adjustments to be made to the components of the cooling systems 132A-N, such as changing fan speeds, decreasing a temperature of the supply air entering the controlled environment 100, increasing an amount of time that the cooling systems 132A-N are operating, etc. The cooling operations can indicate when to schedule cooling cycles in the controlled environment 100 and/or other controlled environments in the facility/system 101. The cooling operations can indicate which pallets to group together for cooling cycles and/or storage in the facility. The cooling operations can indicate flywheeling determinations. The cooling operations can indicate benchmark simulations for the facility.

The control system 116 can transmit the cooling operations to the environmental control unit 118 in block F (146). In some implementations, the control system 116 can generate recommended control operations in block E (144) and transmit those recommendations to the environmental control unit 118. The environmental control unit 118 can then generate control operations based on the recommendations.

The environmental control unit 118 can operate the cooling cycle in the controlled environment 100 according to the transmitted cooling operations (block X, 134). The environmental control unit 118 can, for example, execute instructions to cause one or more adjustments to be made to the operations of the components of the cooling systems 132A-N of the controlled environment 100.

In some implementations, the temperature of the items or other internal conditions of the items that are determined in block D (142) can be transmitted to the user device 117. The user device 117 can then determine the cooling operations based on the temperature of the items (block E, 144). For example, a user at the user device 117 can provide input indicating one or more control operations to be performed at the cooling system 132A-N for the controlled environment 100. Sometimes, the cooling operations can be generated at the control system 116 in block E (144), then transmitted to the user device 117 (F, 146). The cooling operations can be presented as recommended actions in a GUI at the user device 117. The user can then provide input indicating whether or not to implement one or more of the recommended actions. The user device 117 can send instructions to the cooling system 132A-N to operate the cooling cycle in the controlled environment 100 (block X, 134). In some implementations, the user device 117 can send the cooling instructions to the environmental control unit 118 (block F, 146), and the environmental control unit 118 can operate the cooling cycle accordingly (block X, 134). Various other implementations can also be possible for performing the blocks described in FIG. 1B.

Although the disclosed technology is described with regards to modeling temperature conditions of items inside controlled environments, such as blast cells, the same or similar technology can be used for measuring temperature and other ambient conditions in different locations of a facility. For example, the disclosed technology can be used to predict future states (e.g., phase changes) of different types of food products. Sometimes, the disclosed technology can be used to measure current states of the food products. For example, the disclosed technology can be used to measure temperature conditions of items when they are unloaded from delivery trucks to determine where to route the items in the facility (e.g., to storage locations, cold storage locations, controlled environments). As another example, the disclosed technology can be used to measure temperature conditions of items while they are in storage to determine whether the items are being maintained at desired temperatures and/or whether action should be taken to change the temperature conditions of the items. Various other implementations are also possible, including but not limited to using the disclosed technology to more generally model ambient conditions in different locations of the facility

FIG. 2A illustrates an example 201 of the sensor device 106 for measuring conditions around and/or inside a case of items. The configuration of the sensor device 106 described herein can advantageously provide for a completely remote, wireless, battery-operated device having a sensor suite and ability to be removably affixed to any pallets, cases, items, or other components/structures inside a controlled environment or facility more generally.

The example implementation 201 of the sensor device 106 can include a case 209 (e.g., housing, box). A control board 205 can be positioned inside the case 209. The control board 205 can include the wireless transceiver 112 and the power source 114. Sometimes, the control board 205 can include a controller configured to control components of the sensor device 106, such as sensors. The controller can also be configured to transmit signals collected by the sensors of the sensor device to remote computing systems described herein and via the wireless transceiver 112.

The power source 114 can be protected inside the case 209 from ambient conditions external the case 209. Moreover, the control board 205 can generate some amount of heat, which can be transferred to the power source 114 to protect the power source 114 and prevent the power source 114 from dying, losing power or charge, or malfunctioning in different ambient conditions external the case 209. In some implementations, code can be written and implemented that can put the power source 114 in a standby mode until the sensors 108A-N are ready to measure the temperature conditions. The sensors 108A-N can be configured to measure the temperature conditions at predetermined time intervals, such as continuously, every 15 minutes, every 30 minutes, every 1 hour, etc. Continuous measurements of the temperature conditions can be used with the disclosed technology to accurately test and/or iteratively train and improve the models described herein. Whenever the sensors 108A-N are not measuring the temperature conditions, the power source 114 can be turned off or otherwise put in the standby mode. These code operations can therefore preserve the life of the power source 114 and protect the power source from malfunctioning or losing power or charge in different ambient conditions (e.g., below freezing external temperatures).

A plurality of wires 207A-N can be connected to the control board 205 and can extend out from the case 209. At an end of each of the wires 207A-N, a corresponding temperature sensor 108A-N can be attached. In some implementations, one or more of the sensors at the ends of the wires 207A-N can include an acoustic sensor, transducers, or other sensors described herein. In some implementations, the acoustic sensors and/or transducers can be fixedly attached to one or more external surfaces of the case 209. Sometimes, one or more of the plurality of wires 207A-N can be retractable and can go back into the case 209. Any length of the plurality of wires 207A-N can extend out from the case 209 or be positioned inside the case 209. Sometimes, an entire wire including its respective temperature sensor can be retracted back into the case 209. Sometimes, a portion of the wire can be retracted back into the case and another portion of the wire having the respective temperature sensor can extend out from the case 209.

The case 209 can be positioned against or adjacent to any case of items and/or pallet of items in a controlled environment. The wires 207A-N can be configured to extend to multiple desired positions around the case and/or pallet so that temperature signals can be collected at the multiple positions for more accurate temperature readings or environmental conditions in and/or around the case and/or pallet. Sometimes, one or more of the wires 207A-N can be extended between cases on the pallet (e.g., where spacers may be placed between stacked cases) to accurately capture temperature readings or other environmental conditions in and around the many cases stacked on the pallet. The temperature sensors 108A-N can be metal temperature probes, in some implementations. Refer to FIG. 2B below for further discussion about the components of the sensor device 106. For example, the control board can be the same as or similar to integrated circuit 200 in FIG. 2B.

FIG. 2B illustrates another implementation 203 of the example sensor device 106 for measuring conditions around and/or inside a case of items. In the illustrative implementation 203, the sensor device 106 can include a housing 204 (e.g., case, enclosure) having one or more vents 206A-N. The housing 204 can include a bottom surface 210 (e.g., base) with one or more adhesives 208. The adhesives 208 can be configured to removably attach or affix the sensor device 106 to one or more surfaces of pallets, cases of items, and/or items, as described herein. The adhesives 208 can comprise double-sided tape or other similar types of adhesives. In some implementations, the housing 204 may not include the adhesives 208. Rather, the housing 204 can simply be placed on top of a pallet or case. When the sensor device 106 is affixed to the surface of the pallet, case, and/or item, the bottom surface 210 can be adjacent to or otherwise have no gaps between the bottom surface 210 and the surface of the pallet, case, and/or item. The housing 204 can be configured to protect components of the sensor device 106 from ambient conditions, such as extreme temperatures, humidity, condensation etc. The vents 206A-N can be positioned throughout the housing 204 to allow for air to contact the sensors 108, 110, and/or 202 of the sensor device 106. In some implementations, the housing 204 can be shaped as a rectangle. The housing 204 can be sized to fit an integrated circuit 200, and sensors can extend out from the housing 204, as shown and described in reference to FIG. 2A. The housing 204 can, in some implementations, have measurements of approximately 3 in ×3 in×lin. The measurements of the housing 204 can vary based on a size of the integrated circuit 200 and/or other components positioned inside the housing 204.

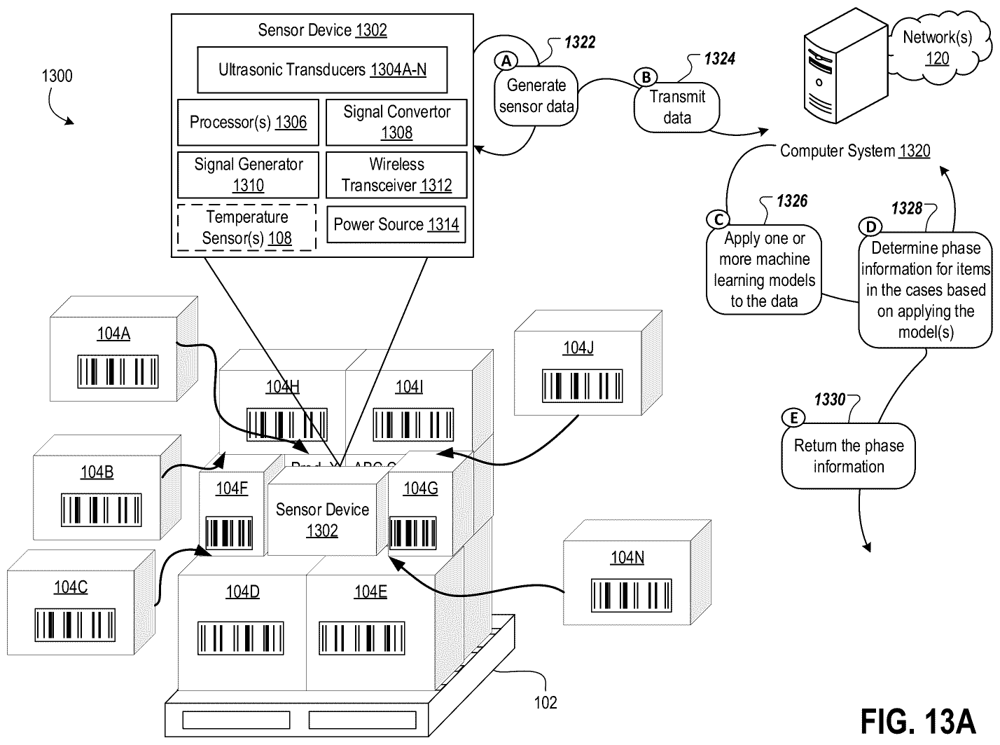

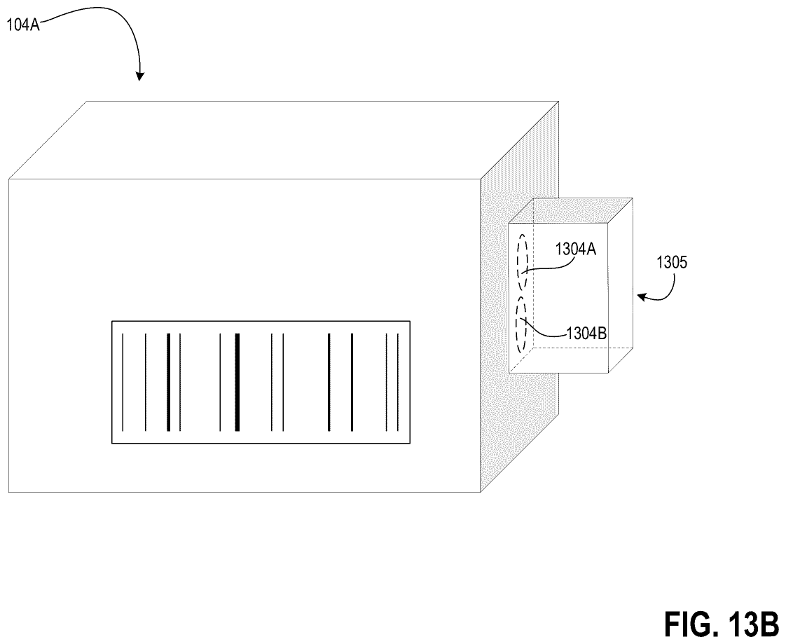

The sensor device 106 can include the integrated circuit 200. The integrated circuit 200 can include the temperature sensor(s) 108, the acoustic sensor(s) 110 (which can include an acoustic or sound generator and an acoustic or sound receiver), the wireless transceiver 112, the power source 114, other sensor(s) 202 and/or ultrasonic transducers 203. The other sensor(s) 202 can include, but are not limited to air flow sensors, humidity sensors, heat flux sensors, etc. The temperature sensor(s) 108 can include, for example, DS18B20 temperature sensors, which can use a supply voltage of approximately 3.0-5.5 V and a current of approximately 1 mA. Refer to at least FIGS. 13A and 13B for further discussion about the ultrasonic transducers 203, which can be configured to generate and receive ultrasonic wave signals. The ultrasonic wave signals can be processed similarly to any of the other signals described herein (e.g., temperature, acoustic, heat flux, humidity) to determine internal conditions of the items being measured (e.g., temperature conditions, phase state, phase change).

Refer to FIG. 2A for further discussion about the power source 114. For example, the components of the sensor device 106 can be rated down to approximately −40° C. In scenarios where the integrated circuit 200 is placed inside the housing 204, temperature inside the housing 204 can remain stable due to heat generated by a processor of the integrated circuit 200. In some implementations, the sensor device 106 can remain inside a controlled environment and operate by collecting sensor signals only when inside the controlled environment. Condensation and/or icing may not be a problem when the sensor device 106 is moved from the controlled environment to a warmer environment, which typically may happen when the sensor device 106 has completed collecting the sensor signals.

The acoustic sensor(s) 110 can, as an illustrative example, have a minimal sampling rate/frequency of approximately 50 kHz. In implementations where the acoustic sensor(s) 110 includes a transducer, the transducer can have a resonance frequency of approximately 25 kHz. The sampling frequency can be larger than or equal to twice the resonance frequency. A signal measured by the acoustic sensor(s) 110 can be approximately +−10 mV. Sometimes, an OpAmp (Operational Amplifier) can be used to amplify the signal that is detected by the acoustic sensor(s) 110. The sensor device 106 can also include an analog-to-digital converter (ADC) whenever analog signals are being measured (e.g., ultrasonic waves with transducers).

As another illustrative example, the acoustic sensor(s) 110 can include an ultrasonic sensor, which can be a combination unit with a receiver and transmitter. The ultrasonic sensor can operate at approximately 48 kHz of voltage and a maximum of approximately 150 Vp-p. The sensor can have a minimum frequency of approximately 46.5 kHz, a typical frequency of 48 kHz, and a maximum frequency of approximately 49.5 kHz. The sensor can have an echo sensitivity of approximately 1 m, and a minimum of approximately 120 mV. In some implementations, the sensor can have a sound pressure level of approximately 30 cm and a sine wave at 10 V, with a minimum of 90 dB. In some implementations, the sensor can operate at a minimum of approximately-40° C. to a maximum of approximately 80° C.

Another illustrative acoustic sensor 110 can operate at approximately 25 kHz of voltage and a maximum of approximately 150 Vp-p. The sensor can have a minimum frequency of approximately 23.5 kHz, a typical frequency of 25 kHz, and a maximum frequency of approximately 26.5 kHz. The sensor can have an echo sensitivity of approximately 1 m, and a minimum of approximately 150 mV. In some implementations, the sensor can have a sound pressure level of approximately 30 cm and a sine wave at 10 V, with a minimum of 110 dB. The sensor can have a detectable range between approximately 0.5 and 15 m. A response time of the sensor can be approximately 2 ms. In some implementations, the sensor can operate at a minimum of approximately −40° C. to a maximum of approximately 80° C.

To test the acoustic sensor(s) 110, an oscilloscope and function generator can be used. A function generator can, for example, generate a square wave pulse with a frequency such as 25 kHz or 48 kHz, depending on the transducer being used. The wave's amplitude can be approximately 5 Vp-p, with a middle of approximately 2.5 V. A resistor can also be used to reduce a current when the transducer discharges. The receiver can be connected to an oscilloscope. When it is not operating, the voltage can be changed. This implementation can be used to measure a signal through air. This implementation can also be used to measure soundwaves through a case (e.g., plastic container) having contents such as water and/or ice. Once the soundwaves are measured, the soundwaves can be further processed to determine a speed of sound. Then, the transducer(s) can be placed on a same side of the case to measure echoes that may occur inside the case when the soundwaves (e.g., ultrasonic sounds) are emitted. Data analysis and processing techniques can then be performed to calculate a distance of each measured echo.

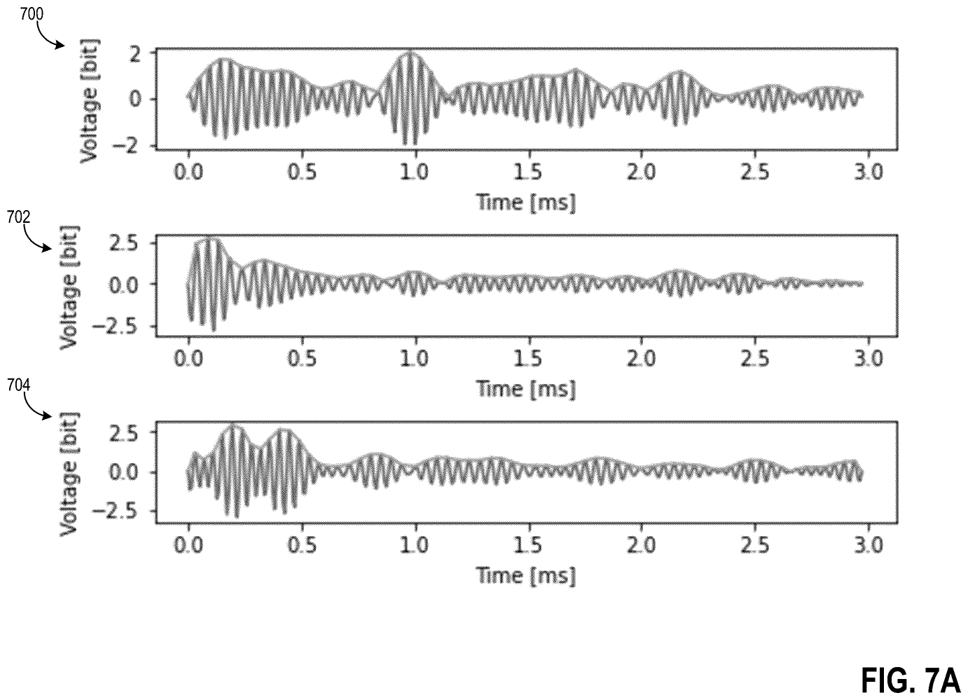

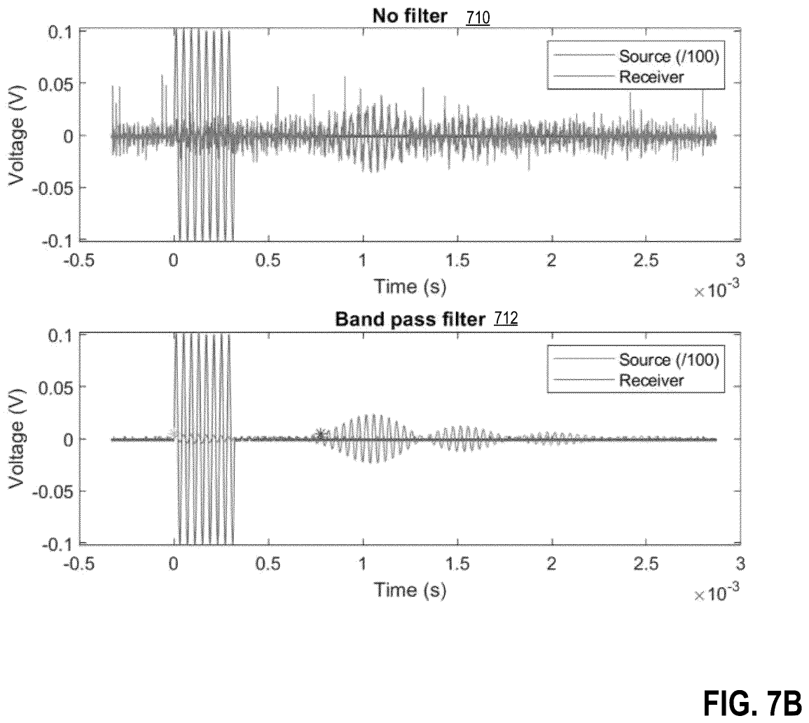

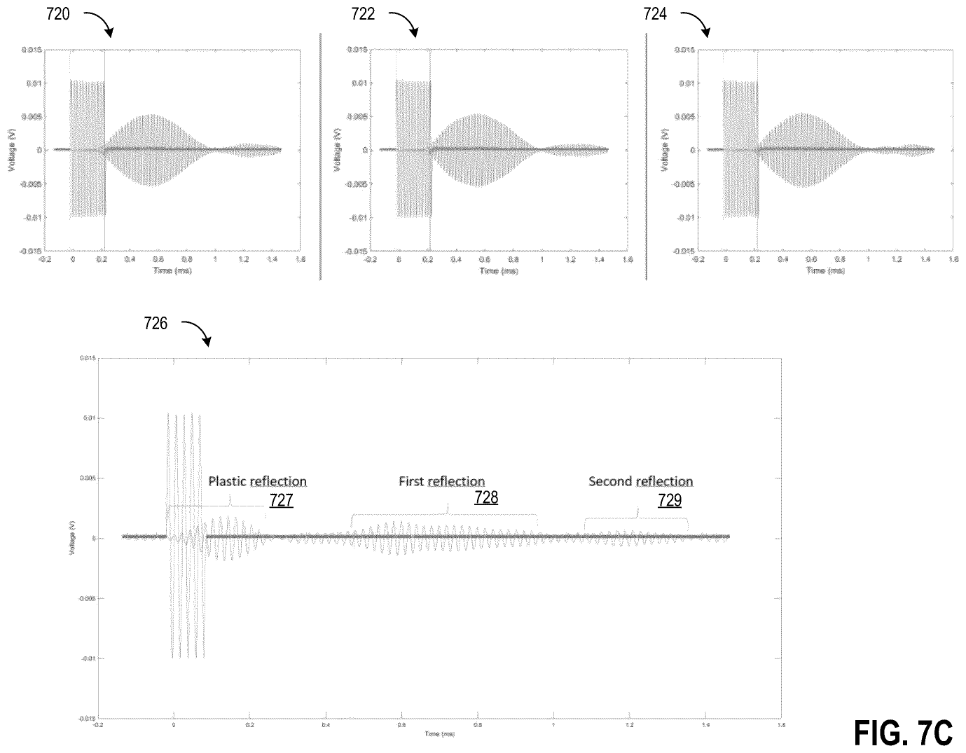

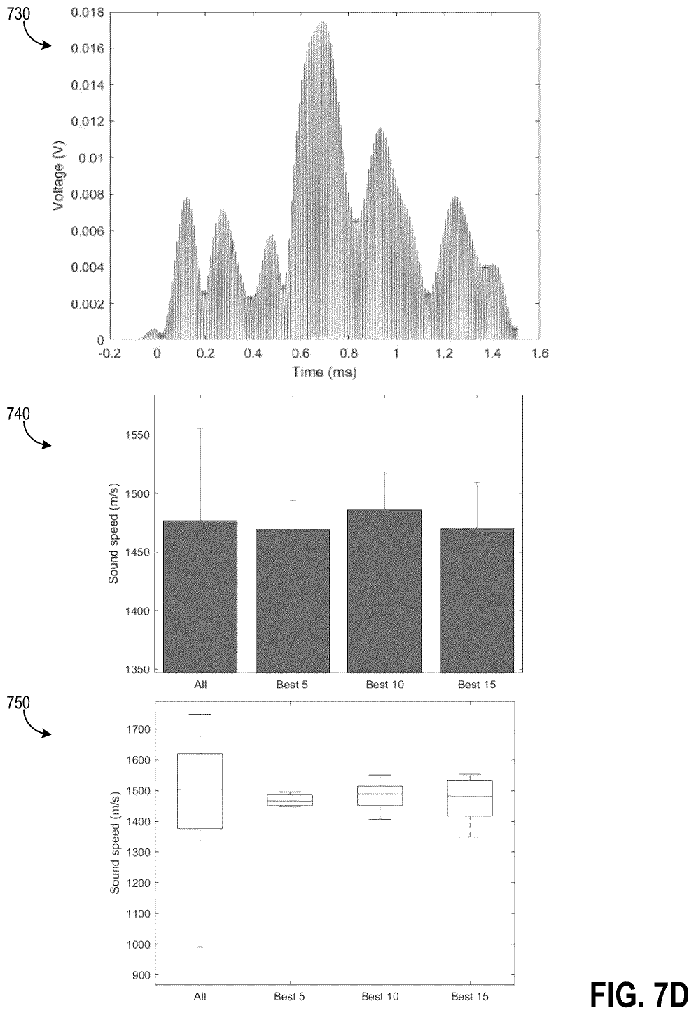

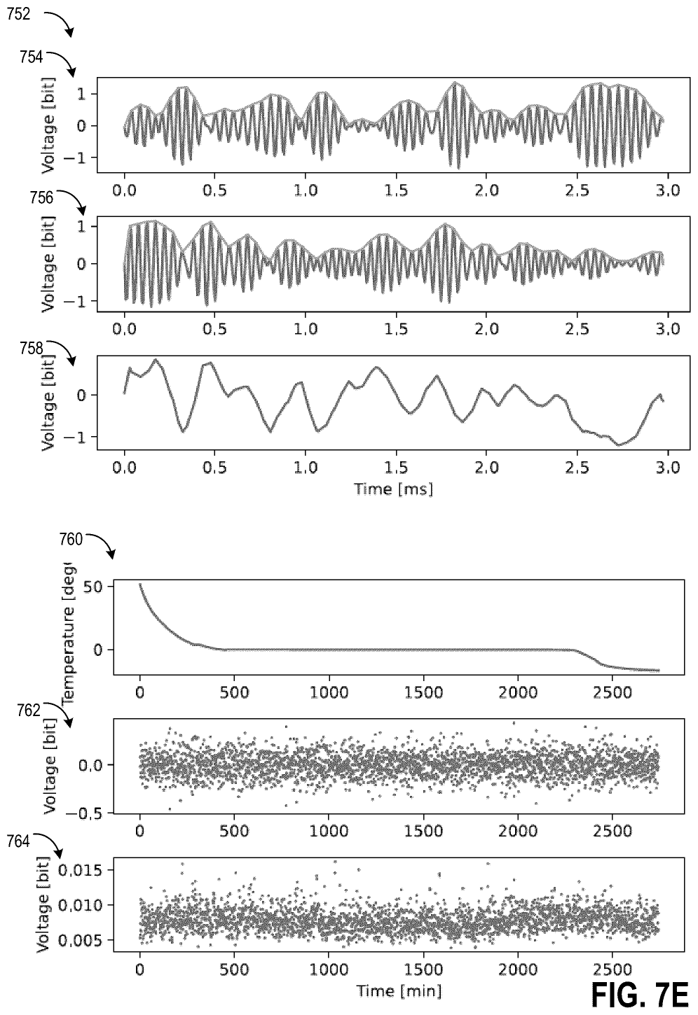

When testing the acoustic sensor(s) 110 used during runtime, one or more parameters can be variated, such as pulse length, pulse amplitude, and/or waveform. The transducers can, for example, be placed on an optical rail. In one implementation, the speed of sound in the air can be determined by measuring a time difference of the pulse. The speed can then be calculated with a formula such as v=x/Δt. A distance x can be variated. With linear regression, the speed can be calculated at each distance. Sometimes, there can be 3 measurements for each distance. In another implementations, the transducers can be placed side by side. A flat surface can be in front of the transducers so that the speed of sound can be calculated with a formula such as v=x/2Δt. The distance x can be variated. With linear regression, the speed can be calculated. At each distance, there can be 3 measurements. Refer to FIGS. 7B, 7C, and 7D for further discussion about the 3 measurements.

FIG. 3 is a conceptual diagram of a system 300 for training at least one model to predict item temperature conditions during a cooling cycle inside a controlled environment, such as the controlled environment 100. The control system 116 can receive training data from a variety of sensors and other sources (e.g., historic data in data stores, warehouse management system data, customer data, pallet data, item data). The control system 116 can receive training data from sensors in a plurality of controlled environments. In the example of FIG. 3, training data is received from sensors in one controlled environment, the controlled environment 100. In the example of FIG. 3, the training data includes temperature data of items in the cases 104A-N on the pallet 102. The training data can include other types of data as well, including but not limited to item type, item weight, expected temperature values for the items (e.g., starting temperature of the items, expected freezing temperature for the items). The training data can include data such as supply and/or return air temperatures inside the controlled environment 100, fan speeds in the controlled environment 100, and other types of facility information.

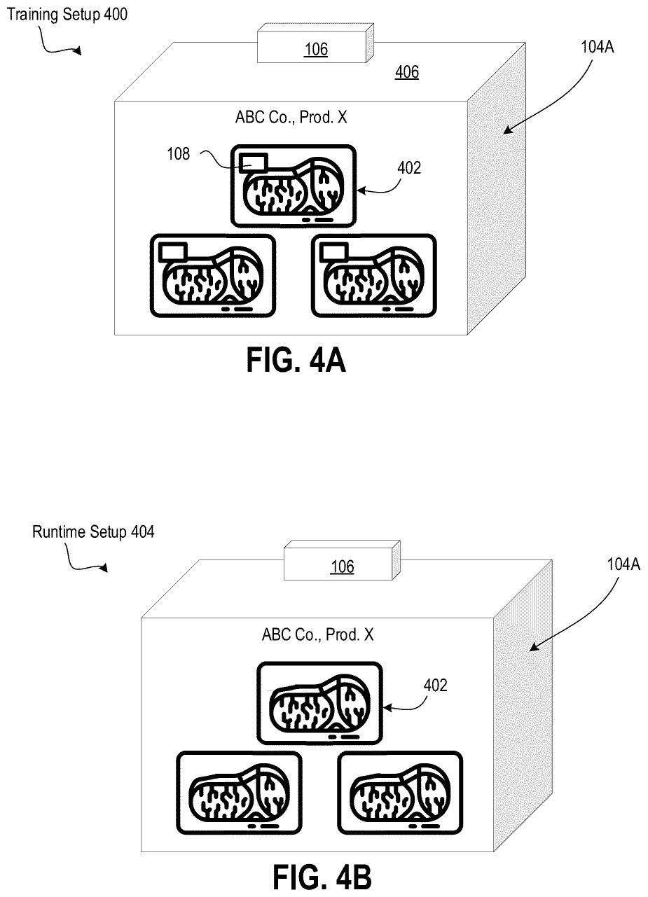

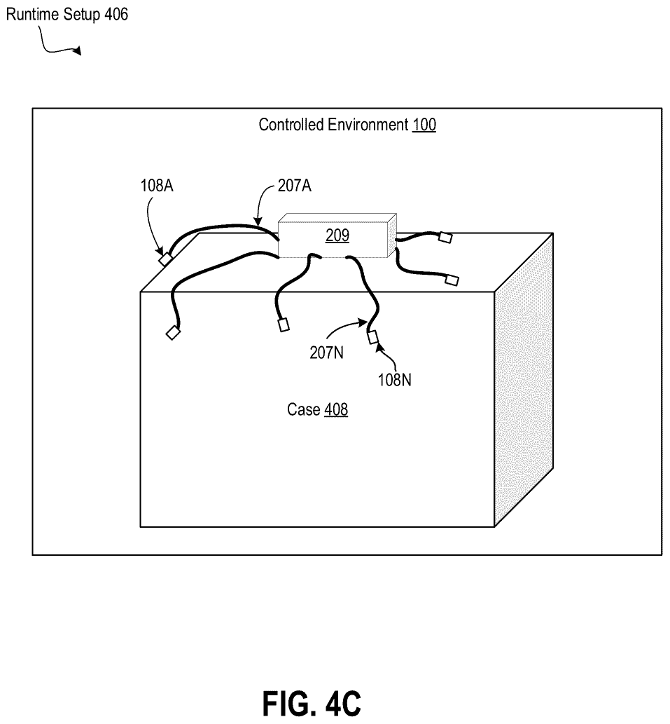

Here, a temperature sensor 108 can be attached to at least one of the cases, such as the case 104B. More particularly, as shown in FIGS. 4A and 4B, the sensor 108 can be a temperature probe that is inserted into the case 104B. The sensor 108 can be inserted into one of the items inside the case 104B. One or more additional sensors can be inserted into one or more of the other cases 104A-N on the pallet. For example, for training purposes, a temperature sensor can be inserted into a case along each side of the pallet 102, such as a left side, a right side, a top, and/or a bottom. As a result, temperature values collected from these sensors can be used to train a model to determine temperature values of items regardless of where the items are positioned on the pallet 102 and relative to cooling components in the controlled environment 100 and/or a location of the supply air.

In the example of FIG. 3, the sensor device 106 can also be used to collect temperature data. The sensor device 106 can be used to collect temperature values outside the cases 104A-N. As a result, the control system 116 can correlate such external temperature values with the internal item temperature values collected by sensors such as the temperature sensor 108.

In some implementations, the control system 116 can also collect temperature, humidity, and other ambient conditions data from the environmental sensors 130A-N inside or surrounding the controlled environment 100. The control system 116 can also correlate the data from the environmental sensors 130A-N with the other temperature data described herein to model internal item temperature values.

In some implementations, a first temperature sensor can be attached to an outside of a case, such as the case 104B, and a second temperature sensor can be injected into one or more items inside the case (e.g., into a core of the item, such as a chicken cutlet). The control system 116 can then train a model using temperature data collected for the one case and the trained model can be used for other cases having same or similar items therein. The trained model can additionally or alternatively be used for cases having various different types of items therein. In yet some implementations, the model can be trained with data from temperature sensors and acoustic sensors described herein.

Still referring to FIG. 3, the sensor device 106, the temperature sensor 108, and/or the environmental sensors 130A-N can collect temperature data in block A (302). The collected temperature data can include ambient temperature values surrounding the cases 104A-N inside the controlled environment 100 when cooling components are activated (e.g., during a cooling cycle) and/or when the cooling components are not activated. The collected temperature data can include internal temperature values of items inside the cases 104A-N when the cooling components are activated and/or deactivated.

The control system 116 can receive the temperature data in block B (304). In some implementations, the control system 116 can receive at least a portion of the temperature data and/or other training data, as described above, from other computing systems (e.g., a warehouse management system, a user device of a facility worker, a customer and/or delivery computing device) and/or a data store.

The control system 116 can apply a physics model to correlate the temperature data from the plurality of sensors described herein (block C, 306). Physics models can allow many parameters to be used to describe a variety of different scenarios all while using the same base model. For example, one of the parameters can be a starting temperature of any product, which can vary depending on the particular product, an origin of the product, customer/producer information, etc. In some implementations, a machine learning model can be used and can be trained to identify and make correlations between different variables.

The control system 116 can train at least one model to predict item temperature (e.g., internal item temperature) based on the correlated temperature data (block D, 308). The control system 116 can build and train one machine learning model for all SKUs, item/food type, and/or packaging type/information. The model can then receive different input parameters during runtime to account for particular item/food types and/or packaging information. In some implementations, the control system 116 can build and train machine learning models for different groups of SKUs, item/food types, and/or packaging type/information. For example, the control system 116 can train a model to predict internal temperature values of solid food items, such as particular types of meats. The control system 116 can train another model to predict internal temperature values of liquids or semi-liquids, such as soups, jams, etc.

Differences in item type and/or packaging can cause cooling cycles to fluctuate for different items, especially during and between phase changes (e.g., liquid to solid for some products such as soups). The model can be trained to correlate item type and/or packaging information (e.g., material type, material thickness, etc.) with temperature data to model internal item temperature values.

In some implementations, the model can also be trained to generate recommendations about when and for how long to pause cooling cycles in the controlled environment 100, when to remove particular item types from the controlled environment 100, and other operations. For example, the model can be trained to identify, based on the temperature data, when the items inside the cases 104A-N are: a particular phase (e.g., solid, liquid), approaching a next phase, undergoing a phase change, and/or a next, subsequent phase. The model can be trained to output the phase of the items. The model can also be trained to generate output indicating operations that can be performed based on the phase of the items. For example, the model may generate recommendations to pause a cooling cycle only after the items have reached a temperature that indicates they have transitioned to a next phase. The model may generate a recommendation to keep the cooling cycle going (do not pause) until the items are detected or predicted, based on their temperature values, as having transitioned to the next phase. If the items, for example, have not yet changed to the next phase, depending on the item type, the items may still be warm or otherwise at a higher risk of spoiling or going bad.

An amount of time that the cooling cycle can be paused can also vary, such as based on the item type and/or packaging information. Pausing the cooling cycle for a couple hours (e.g., 1-3 hours) may not have a significant impact on items that undergo the cooling cycle for extended periods of time (e.g., reach the next phase), such as 24 to 72 hours. The model can be trained to determine a recommended amount of time to pause the cooling cycle based at least in part on the current phase of the items, the next phase of the items, a projected amount of time until the item changes to the next phase, the item type information, and/or the packaging information. A phase change can be a longest amount of time that the items undergo the cooling cycle in the controlled environment 100 (e.g., a phase change can occur for ⅓ to ½ of a total time that the items are in the controlled environment 100). Thus, the model can be trained to determine and recommend pausing times during the phase changes, which can further be dependent on the item type and/or the packaging information.

As an illustrative example, the items can include containers of soup inside of cases that are wrapped in cellophane or a similar plastic-type material/wrapper. Although the soup is a liquid and less dense than other items such as chicken, pork, or fish, the soup can freeze slower than the other items. However, the soup may be less at risk of spoiling than the other items. Furthermore, the wrapper around the cases can cause the cool air to remain inside the cases and slow down a process of defrosting the soup. Taking into consideration all these factors, the model can generate output recommending to pause the cooling cycle for the controlled environment containing the soup for an extended period of time during a phase change and/or once the soup is in the solid phase. Since the cooling cycle can be paused for a longer period of time for the soup than the other items, more energy can be saved and/or repurposed while the cooling cycle for the soup is paused. Operations and/or energy usage inside the facility can be optimized.

As described herein, a fit of the models that are generated and trained can take into account factors such as item type and type of packaging. Customers can provide this type of information to the facility when the items are delivered to the facility for freezing then storage. This information can then be used (e.g., by the control system 116) to adjust parameters of the model during runtime implementation so that the model can efficiently and accurately model internal temperature values for the particular type of items being frozen/cooled.

The control system 116 can then return the model(s) for runtime use in block E (310). Returning the model(s) can include storing the model in a data store until it is later retrieved for runtime use. Returning the model(s) can include locally storing the model at the control system 116 or transmitting the model(s) to one or more other computing systems for runtime use.

FIG. 4A illustrates an example training setup 400 of a case 104A of items, such as an item 402, with sensors used for training at least one model to predict item temperature conditions during a cooling cycle. In the training setup 400, the sensor device 106 having at least one temperature sensor can be affixed/attached to an outer surface of the case 104A, such as a top surface 406. Sometimes, the placement of the sensors may not be important, especially in scenarios where the example sensor device 106 of FIG. 2A is used (e.g., the temperature sensors are on ends of wires that extend from a housing that is placed on the case 104A, where the temperature sensors can be extended to different portions or areas around the case 104A. Placement of the sensors themselves can be more important, which can vary depending on the type of sensor. For example, temperature sensors can be placed anywhere on the outside of the case 104A and/or pallet, out of a direct airflow. Acoustic sensors or transducers, on the other hand, can be affixed to a flat surface of the case 104A so that the transducers can transmit signals through a center of products/items inside the case 104A.

At least one temperature sensor 108 can be a temperature probe that is injected directly into the item 402 inside the case 104A. The item 402 can be a piece of meat. In some implementations, the sensor 108 can be injected into the case 104A so that the sensor 108 is adjacent to or near at least one food item inside the case 104A. The sensor 108 can be injected into any other portion of the case 104A that can provide for accurately measuring a core temperature at a center point of the case 104A.

As described in reference to at least FIG. 3, the sensor device 106 and the at least one temperature sensor 108 can collect temperature data. The temperature data can be training data that is provided to a computer system, such as the control system 116 and used to train at least one model to predict or otherwise determine internal temperature values of the item 402 and other types of items.

FIG. 4B illustrates an example runtime setup 404 of the case 104A of items, such as the item 402, with the sensor device 106 used at runtime to predict item temperature conditions during a cooling cycle. As shown by the runtime setup 404 in FIG. 4B, the temperature sensor 108 may not be injected directly into the items inside the case 104A. Injecting sensors directly into the items inside the case 104A can cause the items to become spoiled or otherwise damaged, which can further cause the items to lower in quality by the time they are delivered to end consumers. The disclosed techniques, on the other hand, advantageously can provide for the internal temperature values of the items to be non-invasively modeled, predicted, or otherwise determined using machine learning models and without causing damage to the items. Accordingly, as shown in FIGS. 4A and 4B, the at least one temperature sensor 108 can be injected into items during a model training phase so that during runtime, sensors may be positioned on or around cases of items instead of injected into the items. Instead, during runtime, the internal temperature values of the items can be modeled with the temperature values detected by the sensors positioned on or around the cases.

FIG. 4C illustrates an example runtime setup 406 of a case of items 408 with a sensor device in the case 209, as shown and described in reference to FIG. 2A, used at runtime to predict internal item conditions during a cooling cycle. As described herein, the case 408 can be placed inside the controlled environment 100 during the cooling cycle. In the runtime setup 406, the plurality of wires 207A-N having the plurality of temperature sensors 108A-N can be extended around edges, sides, surfaces, and/or other portions of the case 409. The case 409 that houses components of the sensor device described herein can be placed anywhere along a top surface of the case 408 since the wires 207A-N with the temperature sensors 108A-N can be extended in any direction to measure ambient conditions surrounding the case 408 in the controlled environment 100. The case 409 having the wires 207A-N and the temperature sensors 108A-N can be placed on an outer surface of the case of items 408 that is away from or opposite a direct airflow (e.g., air that is circulated towards the case of items 408 to freeze the items therein).

In some implementations, the sensor device described herein can be placed on one case of items on a pallet inside the controlled environment 100. For example, the case 409 having the wires 207A-N with the temperature sensors 108A-N can be placed on a case at a center of the pallet that is halfway up a total height of the pallet. As mentioned above, the case 409 can be placed on a side of the case of items that is away from or opposite the direct airflow.

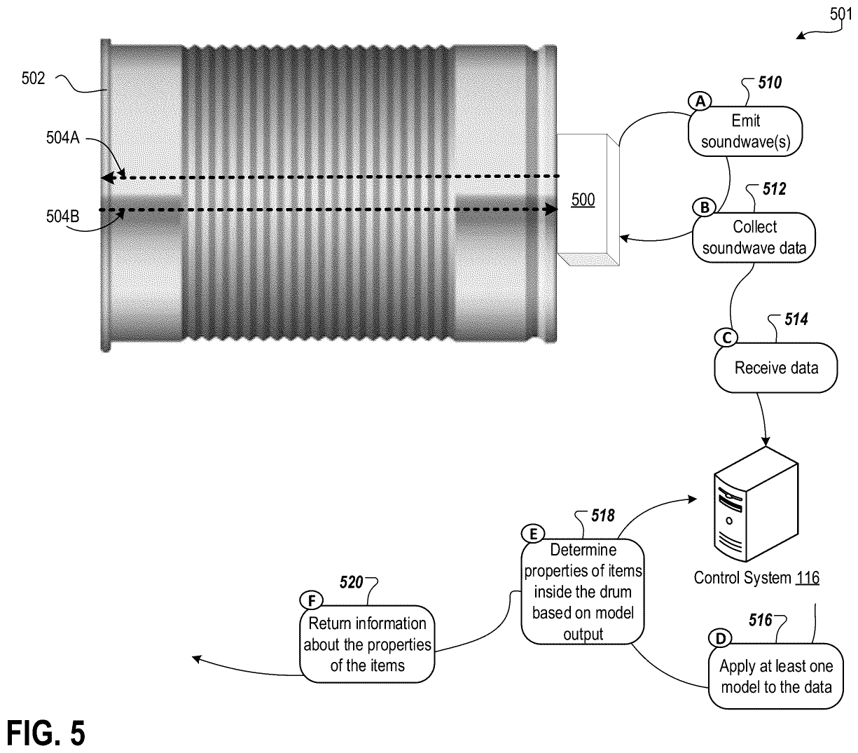

FIG. 5 is a conceptual diagram of a system 501 for modeling item temperature conditions during a cooling cycle inside a controlled environment and based on soundwaves. A drum 502 can contain liquid items/products, such as soup, jams, jellies, or other types of concentrates. The drum 502 can be filled with liquid and may not have air inside (or may have less than a predetermined threshold amount of air inside, such as approximately 2 to 3 millimeters of air within walls of the drum 502 or other container). The system 501 can also be used with other types of containers or cases, such as wax boxes, plastic boxes, or other types of boxes or containers configured to retain liquid items . . .

An acoustic sensor 500, such as a transducer, can be attached to an outer surface of the drum 502. The acoustic sensor 500 can be placed against/on a shortest side of the drum 502 so that emitted soundwaves 504A can be measured along a longest side of the case 104B. Measuring the emitted soundwaves 504A along the longest side of the case 104B can allow for more time to accurately measure an echo 504B of the emitted soundwaves 504A. Sometimes, a film or layer of material can be placed between the acoustic sensor 500 and an outer surface of the drum 502 so that soundwaves 504A emitted by the sensor 500 can penetrate the material of the drum 502 and thus enter the drum 502, bounce off a wall of the drum 502 that is opposite the wall having the sensor 500, and pass though the wall having the sensor 500 to be measured. As an example, the film or layer of material can be a material such as VASELINE.

As another example, multiple acoustic sensors can be placed on a flat surface, either directly across from each other or right next to each other. The acoustic sensors can be oriented such that one or more of the sensors emit signals through a center of the items inside the drum 502. Although not depicted in FIG. 5, another acoustic sensor can be positioned on a surface of the case 502 that is opposite the position of the acoustic sensor 500 or right next to the acoustic sensor 500. In such implementations, the acoustic sensor 500 can be a transducer and the other sensor can be a receiver. In such implementations, the transducer can emit soundwaves (e.g., ultrasonic), which can pass through the drum 502, and be received/detected by the receiver on the opposite surface of the drum 502. The receiver can be configured to collect an echo of the emitted soundwaves, as described above. The multiple sensors can be placed on the same side of the drum 502, then a time that it takes an emitted soundwave to be detected by one of the sensors can be measured and a quantity of reflections in the measured soundwave can be determined (e.g., refer to block D, 516).

In some implementations, the acoustic sensor 500 can be applied to a case in a slowest-freezing position of a controlled environment (e.g., blast cell). As a result, once this particular case is frozen, it can be inferred that all other cases inside the controlled environment (which are not located in the slowest-freezing position) are also frozen.

In the example of FIG. 5, the acoustic sensor 500 can be configured to emit soundwaves 504A that pass through the drum 502, bounce or echo off the surface that is opposite the position of the acoustic sensor 500, and then receive the bounce-back or echo 504B of the emitted soundwaves. As described further in reference to the illustrative examples of FIGS. 7A, 7B, 7C, and 7D, in some implementations, the received soundwaves can be processed to determine a speed of sound. The speed of sound can also be used as an input to determine a phase, temperature, or other properties of the liquid items inside the case 502.

Sound analysis can be performed with the techniques described in FIG. 5 in order to detect presence of ice in food items. This analysis can also be used to reduce over-cooling of the food items and also reduce blast freezer/cooling times. Reducing the cooling times can result in improving efficiency of operations in the facility (e.g., more items can be cooled/frozen in the same amount of time) and/or improving energy consumption and costs (e.g., consuming less energy to cool the same amount of items). At least one model can be trained to analyze various characteristics of soundwaves and correlate such characteristics with properties of the items that are being cooled/frozen. The model can be trained, for example, to determine the items' properties such as current temperature, current phase, and/or percentage of frozen material/content (e.g., percentage of the items that is frozen). As another example, the model can be trained to identify reflections in a soundwave/signal, determine a quantity of the identified reflections, and then correlate the quantity of reflections with a phase of the items.

Accordingly, the acoustic sensor 500 can emit at least one soundwave 504A (block A, 510). The control system 116 can generate a signal and instructions that cause the acoustic sensor 500 to emit the soundwave(s) 504. The sensor 500 can be configured to automatically emit the soundwave(s) 504 continuously and/or at predetermined time intervals (e.g., every 30 seconds, every 1 minute, every 5 minutes, every 1 hour, every 2 hours, every 4 hours, etc.).

Sometime later, the acoustic sensor 500 can collect resulting soundwave data, such as the echo 504B of the emitted soundwave 504A (block B, 512). As described herein, the soundwave(s) 504 can travel through the drum 502 and bounce, reflect, and/or echo off an internal surface of the drum 502 that is opposite the position of the acoustic sensor 500. An amount of time that it takes for the soundwave(s) 504A to bounce back can vary based on how frozen the liquid items inside the drum 502 may be, what type of items are inside the drum 502, a thickness and material type of packaging of the drum 502, a current phase of the items, and/or how frozen or warm the items are. The amount of time that it takes for the soundwave(s) 504A to bounce back can be used as input to the at least one model to determine properties of the items inside the drum 502, such as their internal temperature and/or current phase change.

The control system 116 can receive the soundwave data in block C (514).

The control system 116 can apply at least one model to the data in block D, 516.

In block E (518), the control system 116 can determine a temperature, or other quality/properties of the items inside the drum 502 based on the model output. The model can be trained to determine the properties of the items based on determining speed of sound of the soundwave(s) 504A and correlating the speed of sound with temperature values and/or phases of the items. As described above, the speed of sound can be different based on how quickly or slowly the soundwave 504A bounces back and is received by the acoustic sensor 500. Thus, the speed of sound, further based on the type of item, can indicate whether the items are frozen and/or reached a desired/threshold cooling temperature.

The control system 116 can return information about the determined properties of the items inside the drum 502 (block F, 520). The returned information can include the predicted temperature of the items. The returned information can include a current phase of the items. The returned information can include a projected amount of time that the items are in the current phase, undergoing a phase change, and/or expected to change into the next phase. The returned information can include an expected amount of time remaining until the items reach a desired/threshold cooling temperature. In some implementations, the returned information can be any other output described throughout this disclosure, including but not limited to recommended actions to take during a cooling cycle for the items.

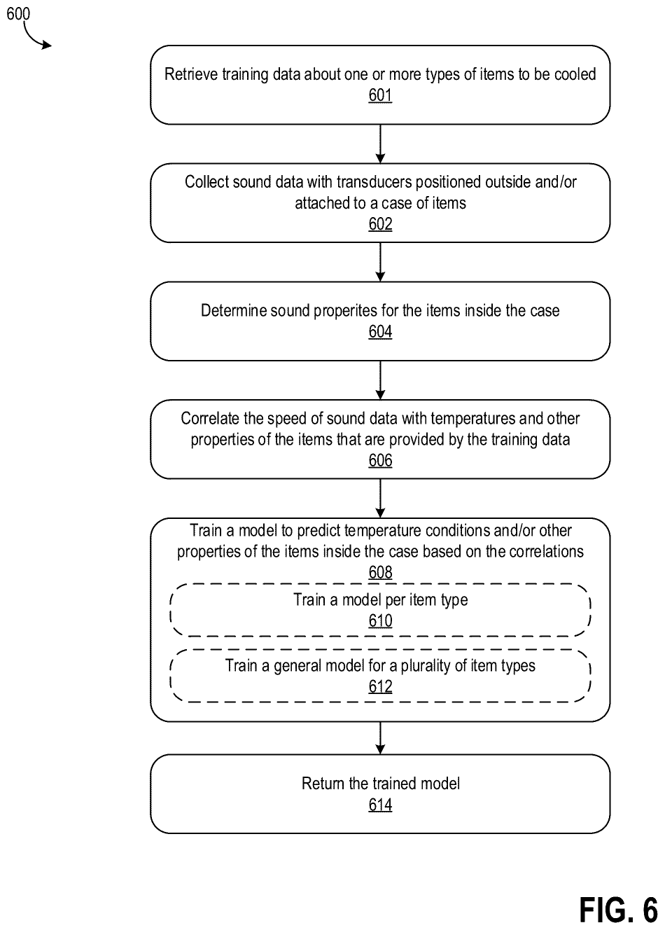

FIG. 6 is a flowchart of a process 600 for training at least one model to predict item temperature conditions during a cooling cycle inside a controlled environment based on soundwaves. The process 600 is described further in reference to FIGS. 5, 7A, 7B, 7C, and 7D.

The process 600 can be performed by the control system 116. The process 600 can also be performed by one or more other computing systems, devices, computers, networks, cloud-based systems, and/or cloud-based services. For illustrative purposes, the process 600 is described from the perspective of a computer system.

Referring to the process 600, the computer system can retrieve training data about one or more types of items to be cooled (block 601). The training data can include but is not limited to temperature values of the items at one or more different phases and/or phase changes, expected cooled temperature values, customer storing information, packaging/case type information, etc.

The computer system can collect sound data with transducers positioned outside and/or attached to a case of items in block 602. In some implementations, one acoustic sensor can be removably affixed to an external surface of the case and used for emitting and receiving soundwaves or others sound signals. In some implementations, a transducer can be removably affixed to a first surface of the case and a receiver can be removably affixed to a second surface of the case that is opposite the first surface. Refer to FIGS. 1B, 2, and 5 for additional discussion about a configuration of the case with one or more acoustic sensors.

In block 604, the computer system can determine sound properties for the items inside the case. For example, the computer system can determine speed of sound data, which can then be used to determine what phase the items are currently in and/or a current internal temperature of the items inside the case. Refer to FIGS. 5, 7A, 7B, 7C, and 7D for further discussion. In some implementations, the computer system can use statistical methods to statistically determine what phases the items are currently at. The statistical methods can include correlating positions and/or TOF info of the collected sound data (e.g., of the echo) with phases of the items. For example, the statistical methods can include determining TOF for each reflection of a soundwave that is collected using the disclosed techniques.

The computer system can correlate the speed of sound data with temperatures and other properties of the items that are provided by the training data (block 606). The computer system can identify associations or correlations between the speed of sound and different internal temperatures or phases of the items inside the case, which can be identified by labeled and annotated training data. For example, the computer system can use temperature probe data from inside the case of items to monitor a progress of a freezing process so that during training, acoustic signals can be correlated with the real-time temperature probe data and phases of the items. The correlation of temperature and acoustic data can be used to further improve the model during iterative training of the model.

The computer system can then train at least one model to predict temperature conditions and/or other properties of the items inside the case based on the correlations in block 608. In other words, the computer system can train the model to determine, based on a speed of sound inside a case of items at any given point during a cooling cycle, an internal temperature of the items inside the case and/or other properties of the items (e.g., a current phase, a percentage of frozen material, a length of time for a phase change, an amount of time remaining in a current phase).

Optionally, the computer system can train a model per item type (block 610). Each model can be trained to correlate properties of the particular item type with speed of sound data for the particular item type. For example, models that process acoustic signals can be generated for particular types of cases that the items may be stored in. The models can be generated for different case dimensions, wax boxes/cases, plastic boxes/cases, metal casings, drums, metal drums, intermediate bulk containers (IBCs), and other types of cases that may be used for specific types of items (e.g., strawberry drums). As another example, models that process temperature signals can be trained more generically so that these models can be applied to various different item types. The model can be trained with a plurality of different parameters, which can be adjusted to fine tune the model for particular implementations/applications. The parameters can include, but are not limited to total mass, item weight, item type, heat capacity, etc. Other parameters are described throughout this disclosure. Any combination of the parameters described herein can be used for training the models and implementing the models during runtime.

Optionally, the computer system can train a general model for a plurality of item types (block 612). The model can include adjustable parameters. A relevant user, or the computer system, can adjust the parameters by providing different inputs based on the particular type of item that is being modeled. The different inputs and/or parameters can include, but are not limited to, specific heat (e.g., how much energy it takes to change a temperature of a particular substance), mass (e.g., amount of the item), case dimensions, starting temperature of the items, etc.

In block 614, the computer system can return the trained model. Returning the model can include storing the model in a data store for later retrieval and use by the computer system. Returning the model can include storing the model locally (e.g., in local RAM or memory) for efficient and fast processing during runtime use.

As described throughout this disclosure, temperature sensors and modeling of temperature signals can be used for determining internal conditions of all types of items, food items, and/or products. If an item is a liquid-based item, then acoustic sensors can additionally or alternatively be used to determine internal conditions of the liquid-based item. In some implementations, signals from both the temperature and acoustic sensors can be used to generate model outcome. Then the model outcome can be correlated to improve model predictions of the internal conditions of the items (e.g., phase state, internal temperature).

FIG. 7A illustrates example soundwaves 700, 702, and 704 measured inside a case of items during a cooling cycle that indicate different phases of the cooling cycle. More particularly, the soundwaves 700, 702, and 704 indicate an echo of a case (e.g., box) filled with a liquid. As shown, the soundwaves 700, 702, and 704 change with each phase and temperature of the liquid as the liquid changes to a solid. The changes in signals of the soundwaves 700, 702, and 704 is in part due to phase changes, during which extra interfaces are created.

In the illustrative example of FIG. 7A, the soundwave 700 can be captured at approximately 100 minutes into the cooling cycle of the liquid inside the case. At this point in time, the liquid is in a liquid phase. The soundwave 702 can be captured at approximately 1,200 minutes into the cooling cycle, during which the liquid is undergoing a phase change to a solid phase. The soundwave 704 can be captured at approximately 2,500 minutes into the cooling cycle, during which the liquid has now fully transitioned into a solid and is in the solid phase (e.g., the liquid is not frozen into ice).

The soundwaves 700, 702, and 704 are generated, captured, and processed using the techniques described at least in reference to FIG. 5. For example, at least one acoustic sensor (e.g., a transducer) can be attached to the case of liquid. The sensor(s) can emit a signal through the liquid (e.g., the liquid can be an item and/or an item like chicken having at least a threshold quantity of liquid inside the case), which can pass through the case and contents therein, bounce off an opposite wall of the case, and be detected by the sensor(s) or another sensor. The sensor(s) can therefore measure an echo taking place inside the case. Data analysis techniques can be performed to calculate a distance of each measured echo. One or more calculations described herein can also be performed to determine a speed of sound corresponding to the echo. The speed of sound can then be correlated to an internal temperature and/or phase of the contents of the case. One or more filtering techniques can also be performed to clean out ambient noise (e.g., fans or other cooling equipment that are operating during the cooling cycle). For example, a band pass filter can be used. In some implementations, one or more different measurements can be averaged to improve a visibility of signals that are detected by the sensor(s).

In some implementations, statistical process control (SPC) techniques can be used to detect out-of-control or abnormalities. This can be done by determining a center line as a mean of all soundwave samples over a time range and upper/lower control limits (UCL, LCL). These can be 3 standard deviations from a norm but also can be 95% CI. With a set of rules, the abnormalities can then be detected. Sometimes, a band-pass filter can be used to filter unwanted noise and artifacts, and a first value of the soundwave signal can be deleted as well. This value can be deleted because the value may often be incorrect (e.g., due to serial reading errors). The cut-off frequencies of the band-pass filter can be approximately 19 kHz and 25 kHz. Because the sampling frequency can be estimated, the best cut-off can be lower than 25 kHz. After these steps, the data can be analyzed with SPC.

For the example soundwaves 700, 702, and 704, an outline of 2 signals (e.g., the line above each of the soundwaves 700, 702, and 704), a different between the soundwaves 700, 702, and 704 can be calculated. A mean and standard deviation (std) can then be calculated for each difference. If the soundwaves 700, 702, and/or 704 are the same, the mean can be 0 and the std can be low. If the soundwaves 700, 702, and/or 704 are different (e.g., due to extra peaks, due to extra interfaces during phase change(s)), the mean can have an offset and the std can be higher. This can provide 2 variables to plot over time in a control chart, namely the mean and the std. A signals outline as shown in FIG. 7A can be determined by finding peaks of the signal. The outline can also be interpolated to achieve an array of a predetermined quantity of data points (e.g., 200 to 400 data points) since the signals may not have a same number of peaks and therefore cannot be subtracted.

A first parameter can be time between subtractions t_{dif}, which can be defined as: (1) subtracting a new sample always from a first measurement, t_{dif}=base and/or (2) subtracting the new sample from a sample some time ago, t_{dif}=#, with a minimal time of 1 minute ago. If the new sample is subtracted from the old sample, t_{dif}=1, then the mean can be around 0 because in 1 minute, not much changes in terms of phase change. There can also be a normal distribution about 0 bits, which can be expected due to some noise in the signal collection. The std can have a skewed normal distribution and/or an unknown distribution at 1 minute.

As mentioned above, a control chart can also be created and used with the disclosed techniques. To create the control chart, the center line (CL), UCL, and LCL can be calculated. The CL can be a mean of the data and/or a moving average or a previous set of the average. The data can be the soundwave signals 700, 702, and/or 704. The UCL and LCL can be calculated with standard deviation of the data and a type 1 error probability. The type 1 error can be a point that is outside the control limits when it is not an error. As an example, with 3.09, the probability of a type 1 error can be approximately 0.0001. UCL can be defined as μ+(3.09)σ and LCL can be defined as μ−(3.09)σ. An upper warning limit (UWL) and a lower warning limit (LWL) ca also be defined to indicate an abnormal soundwave sample, but one that is not out of control. UWL can be defined as μ+(2)σ and LWL can be defined as μ−(2)σ. Another upper control limit (UC) and lower control limit (LC) can also be defined, which can indicate out-of-control situations or abnormalities. UC can be defined as μ+σ and LC can be defined as μ−σ. One or more of the parameters t_{dif}, a method to calculate the mean, and/or a method to calculate the limits can be adjusted or changed when creating and using the control chart.

Each of the limits LCL, LWL, LC, CL, UC, UWL, and UCL can define zones. Illustrative zones can include: Zone++ (voltage>UCL, where UCL can be approximately 0.0250 bits), Zone A+ (UWL<voltage<UCL, where UWL can be approximately 0.0224 bits), Zone B+ (UC<voltage<UWL, where UC can be approximately 0.0180 bits), Zone C+ (CL<voltage<UC, where CL can be approximately 0.0150 bits), Zone C− (LC<voltage<CL, where LC can be approximately 0.0125 bits), Zone B− (LWL<voltage<LC, where LWL can be approximately 0.0090 bits), Zone A− (LCL<voltage<LWL, where LCL can be approximately 0.0055 bits), and Zone−− (voltage<LCL). One or more rules may also be defined and used to detect nonrandom patterns on the control charts described and used with the disclosed techniques. For example, the rules can include, but are not limited to: one or more points being outside of the control limits, 2 or 3 consecutive points being outside 2-sigma warning limits but inside the control limits, 4 or 5 consecutive points beyond 1-sigma limits, approximately 8 consecutive points in one side of the center line, 6 points in a row steadily increasing or decreasing, approximately 15 points in a row in Zone C (both above and below the center line), approximately 14 points in a row alternating up and down, 8 points in a row on both sides of the center line with none in Zone C, an unusual or nonrandom pattern in the data, and/or one or more points near a warning or control limit. The first rule of one or more points being outside of the control limits can be a most important rule. Hence, if errors occur only from the first rule, the errors are more concentrated in an expected position, which is around a time of phase change. With the right setting of the rules, the phase change can therefore be detected. The other rules described above can be designed and used to improve sensitivity of detecting out-of-control points.

FIGS. 7B, 7C, and 7D illustrate example graphs for measuring speed of sound as it travels through cases of items using the disclosed techniques.

FIG. 7B illustrates unfiltered soundwave results 710 and filtered soundwave results 712 using the disclosed techniques. To achieve the results 710 and 712, a setup can include 2 transducers. A function generator can control 1 transducer. The other transducer can be a read-out with an oscilloscope. The transducers can be placed opposite of each other and only a direct signal can be measured, with no reflections in some implementations. The measured direct signal is shown in the results 710.

The measured direct signal can then be filtered with a band-pass filter, as shown in the results 712. The band-pass filter can have a range of approximately 20 kHz to 30 kHz and/or 40 kHz to 50 kHz, depending on a type of transducer being users. A starting point can be determined by a first value that exceeds a threshold of the source, such as 0.05 V (e.g., in FIG. 7B as 0.05/100 V). An endpoint can be a first value that exceeds 0.005 V of the receiver.

The transducers can have different responses to a sine pulse of a resonance frequency. For example, one transducer, the source, can have 3 hills and the other transducer, the receiver, can have 1 damping sinewave. A number of pulses can have an influence on an amplitude of the response. Furthermore, the band-pass filter can be used to clear the data of noise before further being used to determine the speed of the sound.

In some implementations, other techniques can be used than the band-pass filter. For example, an echo of a soundwave can be measured at predetermined time intervals (e.g., every 1 second, 3 seconds, 5 seconds, 10 seconds, etc.), then averaged to a predetermined quantity of minutes (e.g., 1 minute, 2 minutes, 3 minutes, etc.). As an illustrative example, every 5 seconds, an echo can be measured and then all averaged to 1 minute. 12 measurements can be taken and averaged to 1 so that every 1 minute, there's an echo (which can also reduce noise in individual signals). The noise can be reduced significantly, for example with a range of approximately 50 bits before averaging and approximately 10 bits after averaging. Sometimes, instead of 12 measurements, 6 and/or 24 measurements can be taken. The purpose of averaging is to increase the signal-to-noise ratio, while still ensuring the averaging time is not too large. Sometimes, the measured echoes can be averaged to 5 minutes. As another example, other data-cleaning steps can be used. The measurements can be averaged every 1 minute then a band-pass filter can be used with a pass band of approximately 19 kHz to 25 kHz. An outline of the signal can be subtracted from a previous signal (e.g., a signal from approximately 1 to and/or 30 minutes ago). A mean and standard deviation of the subtraction can further be determined (e.g., with control charts from statistical process control),

Averaging the signals can suppress noise and less probable reflections. The band-pass filter can filter the rest of the noise in the signal. When the outline of the signal is subtracted from a signal 1 minute ago, the mean of the subtracted outline may have a normal distribution centered at zero. This can be expected because, in a 1-minute time interval, the echo may not change, due to a timescale of phase change. The standard deviation of the signal may have a skewed normal distribution. This can be expected because there can be a minimum error present in the subtraction. By eye, there may be no visible change in the mean of standard deviation, for the subtraction of the signal with a signal 30 minutes ago. However, the phase change can be detected, with a control chart and Western Electric Rules of statistical process control, for example.

FIG. 7C illustrates soundwaves 720, 722, and 724 that are detected using the disclosed techniques to measure a speed of sound in a container of liquid (e.g., water). The soundwaves 720, 722, and 724 can be generated using different settings for sending a pulse. For example, am amplitude of the pulse can be approximately 5 Vp-p, 10 Vp-p and/or any value in between. A waveform can be a sine and/or square wave. In some implementations, the square wave at 20 Vp-p can be preferred due to higher amplitude of the pulse (which can cause a higher response amplitude). Pulse lengths can also vary, including but not limited to 1, 2, 4, 6, 8, 10, etc. periods. A frequency used can be approximately 25 kHz. In some implementations, the generated soundwaves can also be measured at different distances, such as 0.15, 0.20, 0.30, and/or 0.40 meters.

In the illustrative example of FIG. 7C, the speed of sound in a liquid can be determined using a reflection of the soundwaves on a back wall of the container. A threshold for pulses can also be adjusted to refine calibration of the disclosed techniques. In some implementations, a 25 kHz and/or 48 kHz transducer(s) can be used.

Here, the container can be 21.5 cm by 31.5 cm. The soundwaves 720, 722, and 724 illustrate one-way measurements over 31.5 cm. For data cleaning, a band-pass filter can be applied. The first milliseconds may not be set to zero (since the signal may move too fast), so the pulse and signal can overlap. A mean speed can be 1,485.7 m/s with a standard deviation of 0.5 m/s for these three measurements 720, 722, and 724. In this example, the water used can be tap water and approximately 20° C. A theoretical sound speed can be approximately 1,482 m/s.

A plot 726 illustrates a signal with reflection for the container of liquid. A pulsetrain can travel to one side of the container and then back. A speed of sound calculation can be approximately 1,438.5 m/s, which can be approximately 50 m/s less than the theoretical sound speed. This can be possible because the pulsetrain travels through 0.67 m water and +/−1 cm of plastic (e.g., the material of the container). The speed in plastic can be lower than the speed in the water.

In the plot 726, there are also 3 signals. A first signal 727 can be a possible reflection from the plastic-water interface. A second signal 728 can be a first reflection at a back end of the container (e.g., a surface opposite the surface from which the signal is emitted). A third signal 729 can be a second, last reflection, when the pulse is traveling 4 times through the container. The speed of that pulse can be around 1,280 m/s.

As another illustrative example, a first transducer can be used to send a pulse and a second transducer can be used to receive a pulse. Sometimes, grounding issues can arise, which may cause a leaking signal to a receiver from a source. The leaking signal may have no value to the data. In some implementations, a voltage can be set to 0 V for a duration of the leaking signal (e.g., a first 0.4 ms, because 0.4 ms can be 10 periods of the 25 kHz frequency). Furthermore, during the first 0.42 ms, no signal may be expected due to a minimum time for the pulse to reach the receiver being 0.42 ms at a velocity of v=350 m/s. However, one-way measurements in different phases can have different minimum times. For example, for a one-way measurement in water, a minimum time to reach the receiver from the source can be approximately 0.1 ms over 0.15 m. In ice, as another example, the time for a one-way trip can result in a minimum time of approximately 0.04 ms.

To reduce noise in the signal, a band-pass filter can be used. A bandwidth of the filter can be approximately 24 kHz to 26 kHz, in some implementations.

To determine a TOF, a trigger can be used. The TOF can be calculated with a formula such as TOF=(2*x)/v, where x can be a length of the container and v can be a speed of sound (e.g., 1472 ms⁻¹ at 20° C.). Sometimes, the pulse at the TOF can be a four-way reflection. Six-way and eight-way reflections may also be visible at one or more TOFs.

The TOF can be length of time between a signal being produced and measured. For example, the TOF can be a measure for each reflection of a soundwave. The source, for example, can be above 0.75 V to set a beginning time. That value can be chosen because that can be 3 times a resolution of an oscilloscope that is used with the disclosed techniques. The trigger of the receiver can be approximately 0.002 V, although this may not be the start of the pulse. Generally, this can be a third period that triggered. Therefore, the actual start time can be lower, and can sometimes be corrected by subtracting two periods of approximately 25 kHz.

FIG. 7D illustrates example plots 730, 740, and 750 for measuring a speed of sound through a container of liquid using the disclosed techniques. In the example of FIG. 7D, a pulsetrain used can be approximately 4 to 6 periods, 48 kHz transducer (which can improve accuracy in comparison to a transducer with a lower frequency), and in a square waveform. An amplitude of the pulsetrain can be variated between approximately 5 and 10 Vp-p. Transducers can be placed against a flat container filled with water. A water temperature can be approximately 19.1° C., which means a theoretical sound speed can be 1,479 m/s.

Similar to the example in FIG. 7C, noise reduction techniques can be performed, as shown by plot 730. A voltage can be set to 0 from t=0 until 5 periods of 48 KHz are over. A band-pass filter can then be used to further reduce noise in the signal. An absolute value of the signal response can be captured, as shown in the plot 730. Minimum valleys in the plot 730 can also be defined.

In some illustrative examples, a speed of sound through the water can then be determined. A first minimum can be taken after 0.4 ms. With that time, the speed of sound can be calculated. For example, the speed of sound can be calculated for 26 measurements of the signal. The speed of sound can also be calculated with any other quantity of signal measurements as desired. Plots 740 and 750 illustrate calculated speeds with their 95% confidence intervals. A best 5, 10, and 15 are shown as closest speeds for determining the speed of sound of water in a container to perform the disclosed techniques.

FIG. 7E illustrates an example subtraction method 752 for determining a phase of items based on acoustic signals. The subtraction method 752 includes analyzing a different between two signals shifted in time to detect a phase change. As shown in FIG. 7E, two consecutive signals, 1 minute apart, can be detected, as shown by plots 754 and 756. Bottom plot 758 can show a difference between the signals. When the two signals are both in the same phase, except for a phase change itself, the difference should be zero. When the signals are measured in different phases, the difference should be non-zero because the speed of sound has changed and peak positions may not be aligned. If the difference between two signals is less than 5 minutes, the difference should be zero, since the freezing process is slow. This is because the only reflections present should come from reflections off case walls, which may not change over time.

The difference can be calculated for each pair of samples: for example, if the difference between the signals is 2 minutes, the difference can be calculated as follows, dᵢ fᵢ=sigᵢ−sig₃, dᵢ f₂=sig₂−sig₄, etc. Here dᵢ f is the difference and sig the signal. The difference can be a time-dependent variable (e.g., of approximately 399 samples), shown in the bottom plot 758. Therefore a mean and standard deviation of the difference signals can be calculated, which transforms the difference signal into two variables. When the standard deviation is near zero, the signal difference can be a flat line. When the standard deviation is larger, there may be peaks in the signal difference. These peaks can be formed in several ways: when one signal is measured with a higher temperature, which can lead to shifted two-way, four-way peaks; when one signal is in a phase change, this signal can have more peaks due to extra interfaces (ice-water and water-ice interface). Sometimes, it can be challenging to measure the first way with the setup because the resolution can be too low.

Plot 760 shows an item's core temperature, plot 762 shows a mean of the difference described above, and plot 764 shows a standard deviation of the difference. The time difference between the subtracted signals can be approximately 1 minute. The item can start to freeze at around minute 300 and may be done at approximately 2,300 minutes. The plot 764 can show a dip during a phase change because the signals during the phase change may have more peaks from reflection off water-ice interfaces. These peaks can cancel each other out by accident, sometimes. The same data from the plots 760, 762, and 764 can be used to determine mean and standard deviation of the difference of signals 30 minutes apart. SPC methodology can be used to detect out-of-control or non-random processes and thus to detect times that correspond to start and end times of the phase change (e.g., 300 minute and 2,300 minute).

SPC can be used to detect out-of-control processes or abnormalities. SPC can use control charts for process monitoring. When an unusual event occurs, the measurement may fall outside the control limits, established from previous behavior of the system. In this scenario, when the item's phase starts changing, the signals can be different than when the item was liquid, which will trigger an unusual value. The same holds for when the item stops phase changing. Refer to FIG. 7A for further discussion about the control ranges and limits.

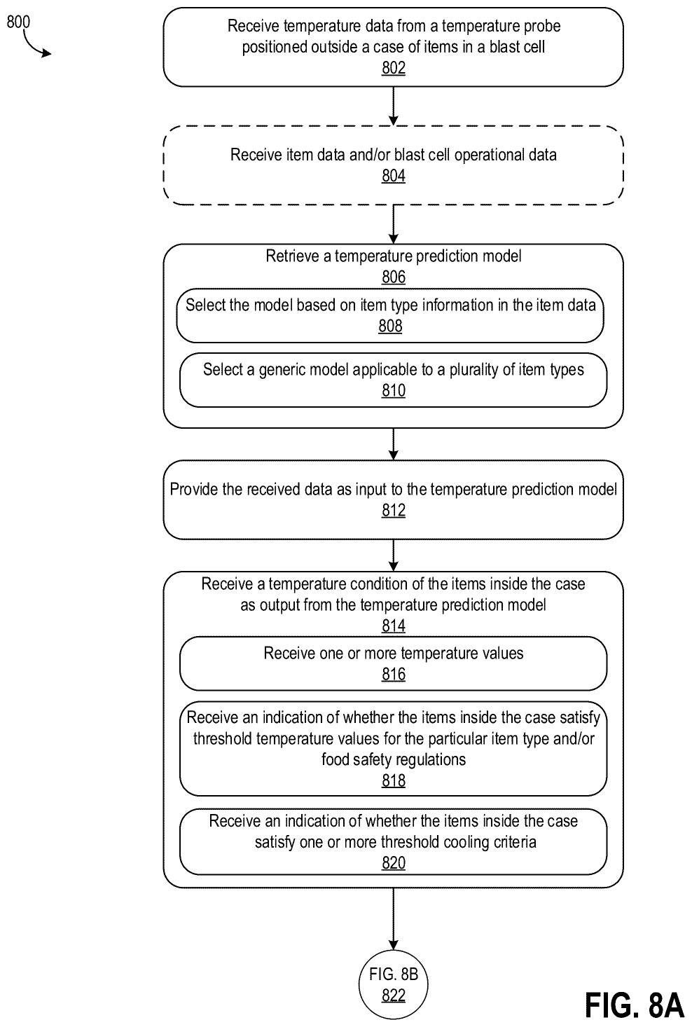

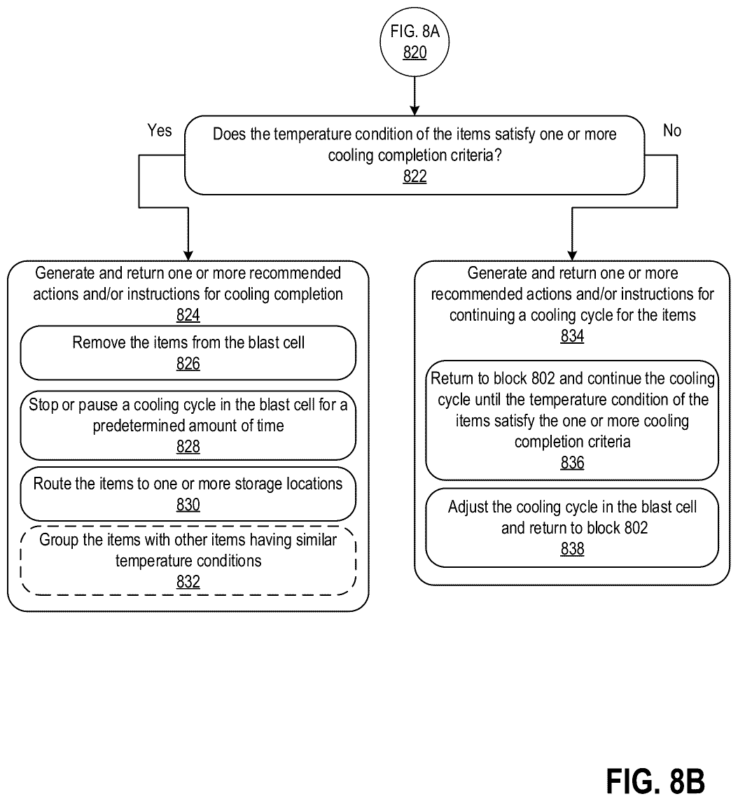

FIGS. 8A and 8B are a flowchart of a process 800 for adjusting a controlled environment based on modeling item temperature conditions inside the controlled environment during a cooling cycle and using temperature data. The process 800 can be performed by the control system 116. The process 800 can also be performed by one or more other computing systems, devices, computers, networks, cloud-based systems, and/or cloud-based services. For illustrative purposes, the process 800 is described from the perspective of a computer system.

Referring to the process 800 in FIGS. 8A and 8B, the computer system can receive temperature data from a temperature probe positioned outside a case of items in a blast cell (block 802). The blast cell can be a controlled environment, such as the controller environment 100 described herein. Refer to at least FIG. 1A for further discussion.

The computer system can optionally receive item data and/or blast cell operational data in block 804. The item data and/or blast cell operational data can include supply air temperature in blast cell, return air temperature from the blast cell, type of item inside the cases, type of case material/packaging, and other types of data described throughout this disclosure.

In block 806, the computer system can retrieve a temperature prediction model. The computer system can select the model based on item type information in the item data (block 808). Additionally or alternatively, the computer system can select a generic model applicable to a plurality of item types (block 810). By selecting the generic model, the computer system can also provide inputs to adjust parameters of the model so that the model can accurately predict temperature conditions of the particular items in the case. The parameters can include, for example, item type information and/or case material, type, and/or packaging information. Various other parameters can also be defined during training and used to fit the model to the particular runtime application.

The computer system can provide the received data as input to the temperature prediction model in block 812. The computer system can provide any portion of the received data as model input. For example, the computer system may only provide the temperature data from the temperature probe as input. The computer system can additionally or alternatively provide temperature data from environmental sensors inside the blast cell as input. In some implementations, the item data and/or the blast cell operational data can be provided as inputs to the model and/or as inputs for adjusting the parameters of the model.

In block 814, the computer system can receive a temperature condition of the items inside the case as output from the temperature prediction model. For example, the computer system can receive one or more temperature values in block 816. Thus, the temperature condition can indicate a projected/modeled current internal temperature of the items inside the case. The temperature condition can indicate a projected/modeled current phase of the items inside the case. The temperature condition can indicate a projected/modeled phase change of the items inside the case.

As another example, the computer system can receive an indication of whether the items inside the case satisfy threshold temperature values for the particular item type and/or food safety regulations (block 818). The threshold temperature values for the particular item type and/or the food safety regulations can be received in block 804. The threshold temperature value(s) can indicate a desired frozen temperature for the particular item type. The threshold temperature value(s) can be defined by a customer and/or provider of the items inside the case. The threshold temperature value(s) can be a desired temperature of the items during a phase change, which can indicate whether the items are being cooled/frozen at a desired speed.

As yet another example, the computer system can receive an indication of whether the items inside the case satisfy one or more threshold cooling criteria (block 820). The one or more threshold cooling criteria can indicate desired temperatures and/or desired amounts of time for the particular items to remain in a particular phase, for the particular items to start a phase change, for the particular items to undergo the phase change, and/or for the particular items to reach a next phase. In some implementations, the threshold cooling criteria can include one or more ranges for phases, time in controlled environments, and/or desired temperature. Various other conditions can also be determined by the model output, as described herein.

In block 822, the computer system can determine whether the temperature condition of the items satisfy one or more cooling completion criteria. The cooling completion criteria can be the same as or different from the threshold cooling criteria in block 820. The cooling completion criteria can, for example, indicate a desired cooled/frozen temperature for the particular items. Once the desired temperature is reached (and/or maintained for at least a threshold amount of time), the items can be removed from the blast cell and moved to one or more storage locations in the facility.

If the cooling completion criteria is/are satisfied, the computer system can proceed to block 824, in which the computer system can generate and return one or more recommended actions and/or instructions for cooling completion. In other words, the computer system can determine and/or recommend actions to end the cooling cycle for the particular items. In some implementations, the computer system can send a notification to one or more other computing systems to generate and return the recommended actions and/or instructions for cooling completion. The recommended actions can include, but are not limited to, removing the items from the blast cell (block 826), stopping or pausing the cooling cycle in the blast cell for a predetermined amount of time (block 828), and/or routing the items to one or more storage locations (block 830). Optionally, the recommended actions can include grouping the items with other items having similar temperature conditions (block 832).

In some implementations, recommending actions to stop and/or pause the cooling cycle can include determining how to shift an amount of time that the items remain inside the blast cell and continue through the cooling cycle once the cycle is resumed. Flywheeling technology can also be used to determine when to defrost and/or frost the items inside the blast cell before, during, and/or after the cooling cycle is stopped or paused. The flywheeling technology can be used in combination with the disclosed techniques to determine when and how to shift energy consumption and operation of the blast cell. As a result, energy can be saved and used efficiently across operational tasks being performed in the facility. Decisions about whether to stop or pause the cooling cycle can also vary depending on the food safety regulations and whether bacteria may grow or otherwise cause food quality issues in the items inside the blast cell. Whether the cooling cycle can be paused and for how long may depend on the particular item type in the blast cell.

If the one or more cooling completion criteria is not satisfied in block 822, the computer system can generate and return one or more recommended actions and/or instructions for continuing the cooling cycle for the items (block 834). For example, the computer system can return to block 802 and continue the cooling cycle through the process 800 until the temperature condition of the items satisfy the one or more cooling completion criteria (block 836). As another example, the computer system can generate recommended actions to adjust the cooling cycle in the blast cell in block 838. In some implementations, the computer system can also return to block 802 and continue through the process 800 as described above. The cooling cycle can be adjusted by increasing a fan speed (which can cause the items inside the blast cell to be cooled/frozen faster), decreasing a temperature of the supply air entering the blast cell, activating additional fans inside the blast cell to circulate the cooled air more quickly around the cases of items stacked therein, and/or performing other operational adjustments to refrigeration and/or cooling systems that are used in the blast cell. In some implementations, the computer system can send a notification to one or more other computing systems to generate and return the recommended actions.

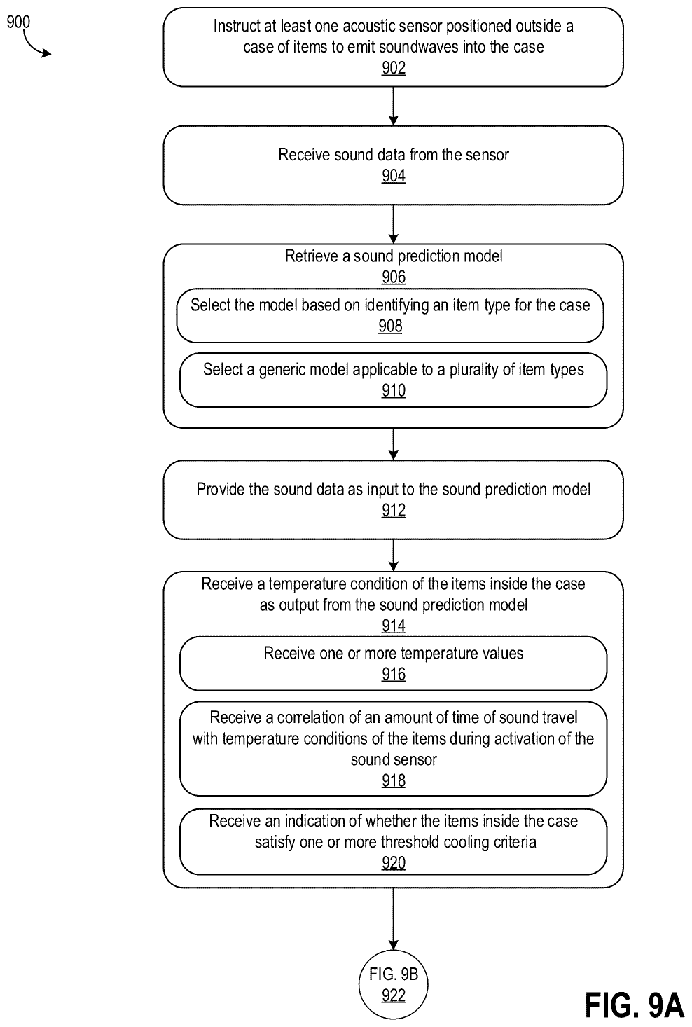

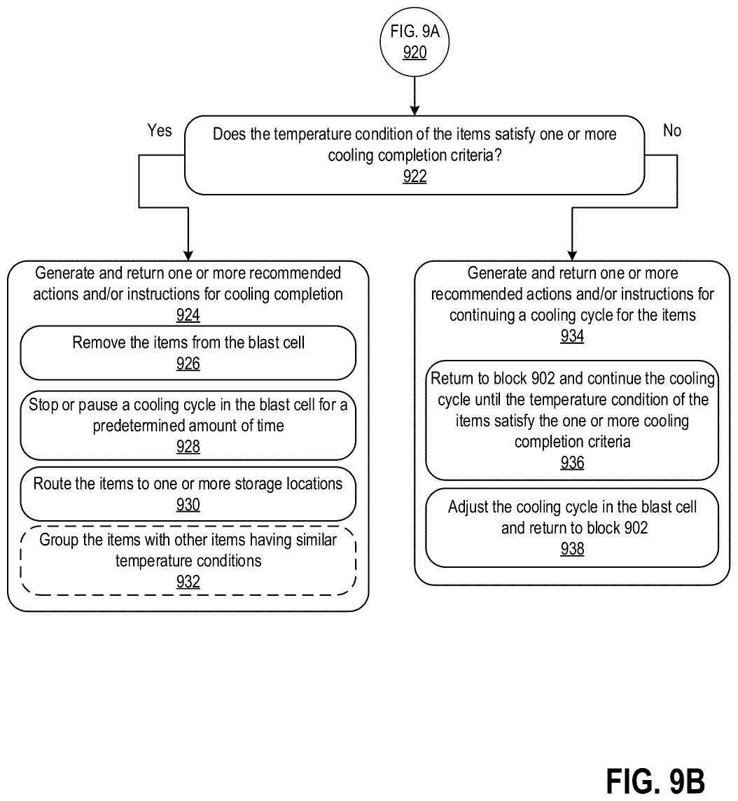

FIGS. 9A and 9B are a flowchart of a process 900 for adjusting a controlled environment based on modeling item temperature conditions inside the controlled environment during a cooling cycle and using sound data. Sometimes, both the processes 800 (refer to FIGS. 8A and 8B) and 900 can be performed to determine the item conditions and recommend adjustments for the controlled environment. Both the process 800 and 900 can be used to check/verify determinations that are made from each of the processes. Temperature data collected and modeled in the process 800 can beneficially provide information about core temperature of the items and how far along the items are in a phase change. Acoustic sound data collected and modeled in the process 900 can beneficially provide information about how far along the items are in a phase change and thus act as a time indication of the phase change for the items. The combination of the process 800 and 900 can provide accurate and robust information about the items during the cooling cycle.

Sometimes, the process 900 may be used based on a type of the items inside the controlled environment. For example, if the items are liquid-based items, then the process 900 can be used (alone or in combination with the process 800). On the other hand, for example, if the items are solid-based, like vegetables or meats that are sealed without any liquids, then the process 800 may be performed instead of the process 900. The process 900 may be performed when sound data can travel and be measured through quantities of liquid in the controlled environment.

The process 900 can be performed by the control system 116. The process 900 can also be performed by one or more other computing systems, devices, computers, networks, cloud-based systems, and/or cloud-based services. For illustrative purposes, the process 900 is described from the perspective of a computer system.

Referring to the process 900 in FIGS. 9A and 9B, the computer system can instruct at least one acoustic sensor positioned outside a case of items to emit soundwaves into the case (block 902). The sensor can be a transducer. The sensor can be a sound probe. Refer to FIGS. 2, 5, 6, 7A, 7B, 7C, and 7D for further discussion.

The computer system can receive sound data from the sensor in block 904. The sound data can include, for example, ultrasound waves, echoes, or other reflections of the emitted sound pulse/signals that bounces off edges, walls, or other surfaces inside the case. Refer to at least FIGS. 7A, 7B, 7C, and 7D for further discussion. In some implementations, as described in reference to block 804 in the process 800 of FIG. 8A, the computer system can also receive item data and/or blast cell operational data. This data can be used by the computer system as additional inputs to a model. This data can be used to adjust and/or fit parameters of the model for the particular type of items inside the case.

The computer system can retrieve a sound prediction model in block 906. For example, the computer system can select the model based on identifying an item type for the case of items (block 908). As another example, the computer system can select a generic model applicable to a plurality of items types (block 910). Refer to blocks 806-810 in the process 800 of FIG. 8A for further discussion.

In block 912, the computer system can provide the sound data as input to the sound prediction model. The sound data can include one or more signals representing the detected soundwaves. The sound data can include an amount of time that it took for the emitted soundwave(s) to travel through the case of items, bounce off a far edge of the case, and return to a position of the sensor. The sound data can include an amount of time that it took for the emitted soundwave(s) to travel through the case and be received by another sensor positioned on the opposite side of the case from the position of the sensor that emitted the soundwave. Refer to at least FIGS. 7A, 7B, 7C, and 7D for further discussion about the sound data.

The computer system can receive a temperature condition of the items inside the case as output from the sound prediction model (block 914). For example, the computer system can receive one or more temperature values in block 916. Additionally or alternatively, the computer system can receive a correlation of an amount of time of sound travel with temperature conditions of the items during activation of the sound sensor (block 918). Additionally or alternatively, the computer system can receive an indication of whether the items inside the case satisfy one or more threshold cooling criteria (block 920). Refer to at least FIGS. 2, 5, 6, 7A, 7B, 7C, and 7D for further discussion. Furthermore, refer to blocks 814-820 in the process 800 of FIG. 8A for further discussion about the temperature condition.

In block 922, the computer system can determine whether the temperature condition of the items satisfy one or more cooling completion criteria. For example, if an amount of time for being inside the blast cell during the cooling cycle exceeds a threshold value, then the items may be cooled/frozen enough. If the amount of time is less than the threshold value, then the items may not be cooled/frozen enough. As another example, if the temperature of the items is within a desired temperature range, then the items may be cooled/frozen enough. If the temperature of the items is greater than the desired temperature range, then the items may be frozen/cooled too much. The cooling completion criteria can provide a sliding scale and/or range of temperature values and/or cooling time to be used by the computer system to determine whether the items have been cooled enough during the cooling cycle in the blast cell. Refer at least to block 822 in the process 800 of FIG. 8B for additional discussion.

If the one or more cooling completion criteria is satisfied, the computer system can generate and return one or more recommended actions and/or instructions for cooling completion (block 924). For example, the recommended actions can include, but are not limited to, removing the items from the blast cell (block 926), stopping or pausing the cooling cycle in the blast cell for a predetermined amount of time (block 928), routing the items to one or more storage locations (block 930), and/or optionally grouping the items with other items having similar temperature conditions (block 932). Refer to blocks 824-832 in the process 800 of FIG. 8B for further discussion.

Referring back to block 922, if the one or more cooling completion criteria is not satisfied, the computer system can generate and return one or more recommended actions and/or instructions for continuing the cooling cycle for the items in block 934. For example, the computer system can return to block 902 and continue the cooling cycle in the process 900 until the temperature condition of the items satisfy the one or more cooling completion criteria (block 936). As another example, the computer system can recommend actions to adjust the cooling cycle in the blast cell and then return to block 902 and iterate through the process 900 as described herein. Refer to blocks 834-838 in the process 800 of FIG. 8B for further discussion.

FIGS. 10A and 10B illustrate example system components that can be used to perform the disclosed techniques. Referring to both FIGS. 10A and 10B, the control system 116, the sensor device 106, a data store 1002, user device(s) 1000, an environmental control unit 118, the cooling systems 132A-N, the environmental sensors 130A-N, and a sensor device 1302 can communicate (e.g., wired and/or wirelessly) via the network(s) 120.

The control system 116 can include processor(s) 1004, a communication interface 1006, a model training engine 1008, an item condition determiner 1010, an environmental control engine 1012, a blast scheduling engine 1014, an item routing determiner 1016, an optional item grouping determiner 1018, a blast flywheeling determiner 1020, and/or an output generator 1022. The processor(s) 1004 can be configured to perform one or more operations described herein or otherwise execute instructions that cause components of the control system 116 to perform the operations. The communication interface 1006 can be configured to provide communication between the components of the control system 116 and other system components described herein.

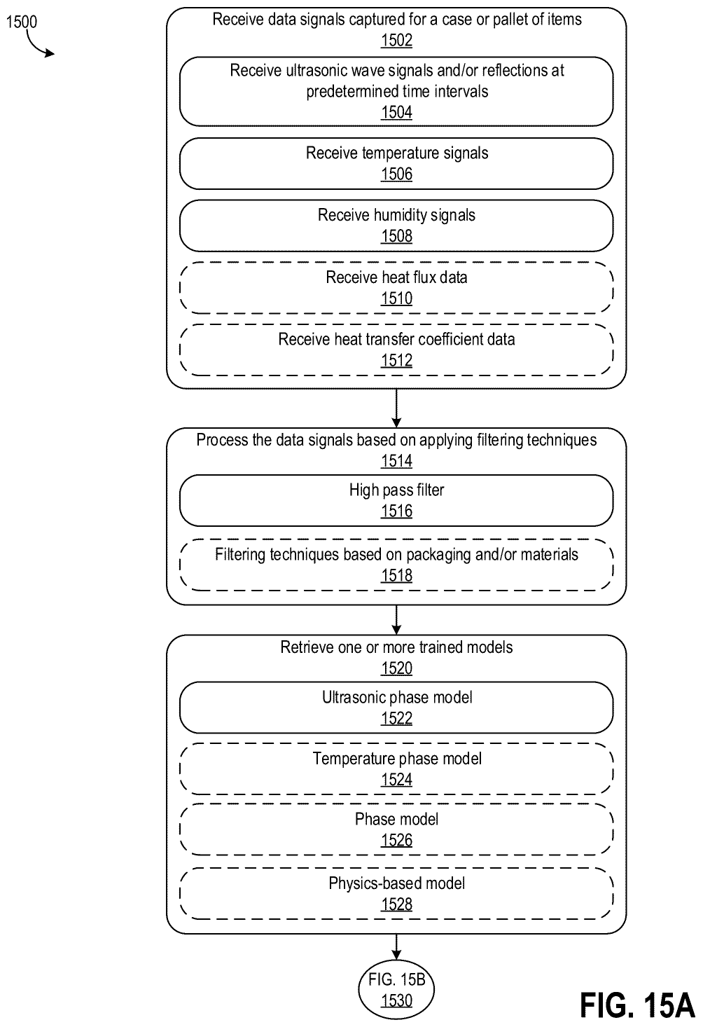

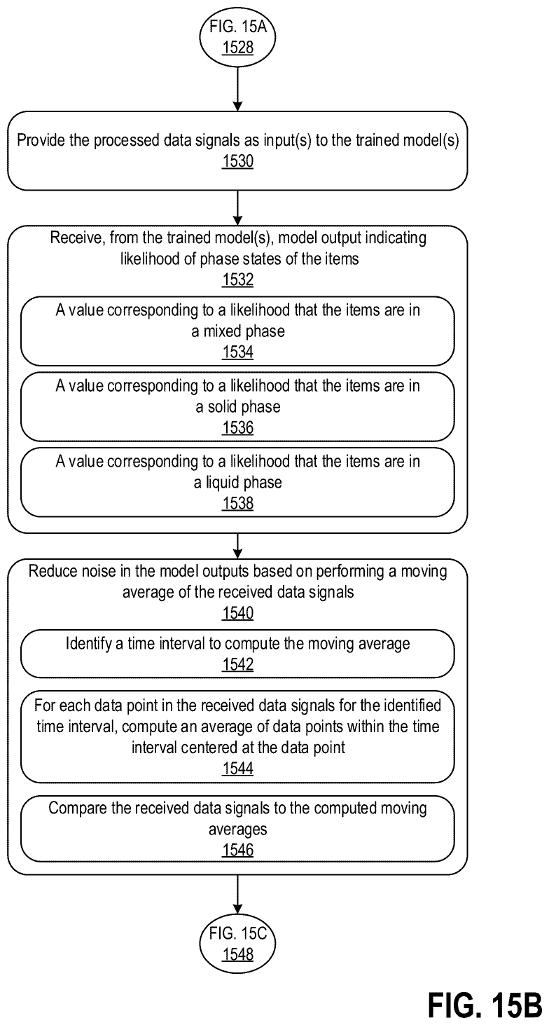

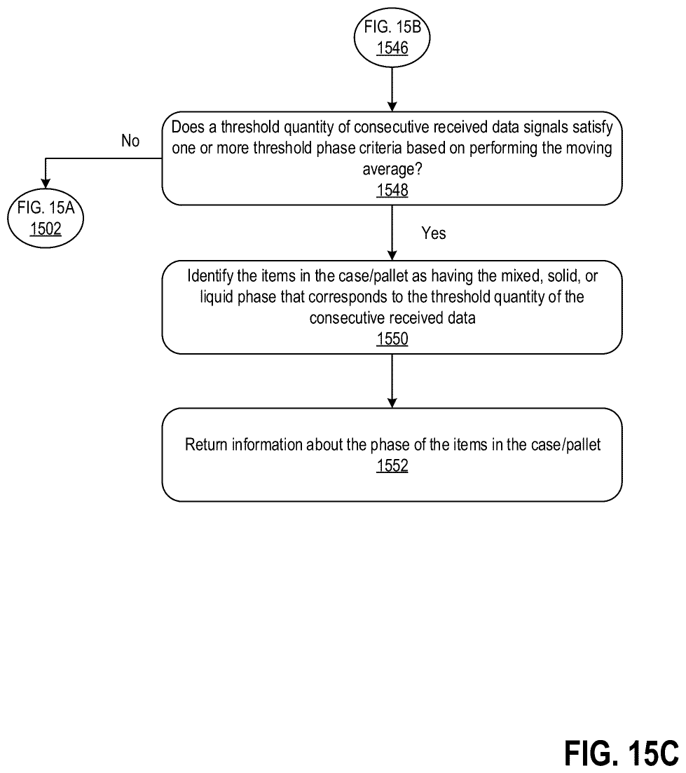

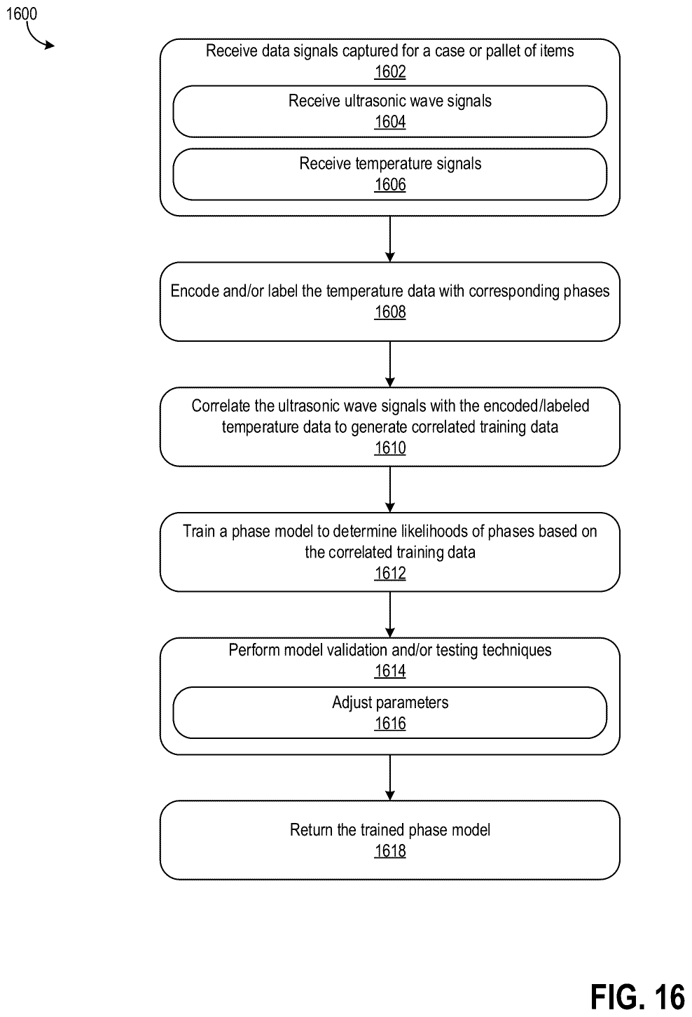

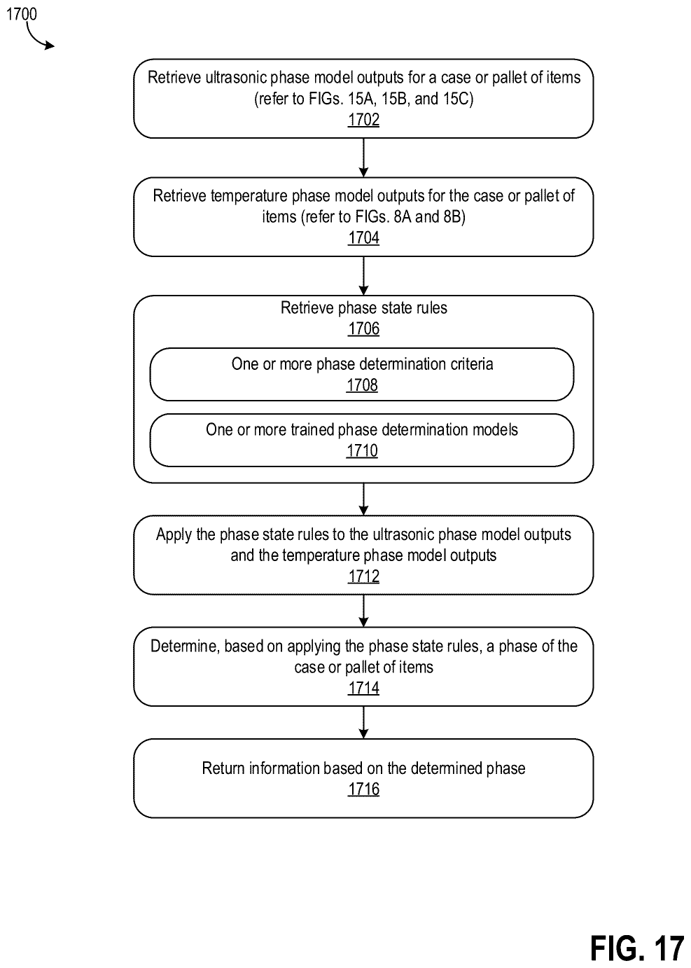

The model training engine 1008 can be configured to generate, train, and iteratively train/improve any of the models described herein, such as item-type temperature models 1026A-N, a generic temperature model 1028, item-type sound models 1030A-N, and a generic sound model 1032. The engine 1008 can retrieve training data 1024 from the data store 1002. The engine 1008 can also retrieve other data from the data store 1002 for purposes of training the models, such as item data 1034, cooling environment schedules 1044A-N, cooling criteria 1036A-N, food safety regulations 1038A-N, facility data 1040, and/or cooling environment operational data 1042A-N. Refer to FIGS. 3, 4A, 4B, and 6 for further discussion about training the models described herein. The model training engine 1008 may also be configured to train one or more phase state models 1025A-N. The phase state models 1025A-N can be trained to determine likelihood of the items being at one or more phases (e.g., liquid, solid, mixed). Refer to at least FIGS. 15A, 15B, 15C, and 16 for further discussion.

The item condition determiner 1010 can be configured to apply one or more of the models 1025A-N, 1026A-N, 1028, 1030A-N, and 1032 to sensor data generated by the sensor device 106 and/or the environmental sensors 130A-N to determine a temperature of items inside the controlled environment 100 during a cooling cycle and/or a phase state of the items. The determiner 1010 can be configured to determine one or more other conditions of the items, including but not limited to a freeze percentage, a current phase, an upcoming phase, an amount of time in a current phase or undergoing a phase change, a food safety indication, etc. Refer to at least FIGS. 1B, 5, 8A, 8B, 9A, 9B, 15A, 15B, 15C, and 17 for further discussion.

The environmental control engine 1012 can be configured to generate one or more controls or adjustments to controls/operations of the cooling system 132A-N. Such controls and/or adjustments can be generated based on the item conditions generated by the item condition determiner 1010. Such controls and/or adjustments can also be generated based on the item data 1034, the cooling environment schedules 1044A-N, the cooling criteria 1036A-N, the food safety regulations 1038A-N, the facility data 1040, and/or the cooling environment operational data 1042A-N. The controls and/or adjustments can further be generated based on sensor data detected by the environmental sensors 130A-N.

In some implementations, the engine 1012 can generate a recommendation for adjusting the operations of the cooling system 132A-N. The recommendation can then be transmitted to the environmental control unit 118, which can generate one or more adjustments to the operations of the cooling system 132A-N. Refer to at least FIGS. 1A, 1B, 8A, 8B, 9A, and 9B for further discussion about generating the adjustments and/or controls for the cooling system 132A-N.

The blast scheduling engine 1014 can be configured to generate and/or modify the cooling cycles (e.g., blast schedules) for one or more of the controlled environments 100 in a facility 1068. The engine 1014 can use any determinations made by the item condition determiner 1010 and/or the environmental control engine 1012 to generate and/or modify the cooling cycles. The engine 1014 can also receive sensor data from the environmental sensors 130A-N and/or the sensor device 106, which can be used to generate and/or modify the cooling cycles. In some implementations, the engine 1014 can generate and/or modify the cooling cycles using one or more data retrieved/accessed from the data store 1002, such as the item data 1034, the existing cooling environment schedules 1044A-N, the cooling criteria 1036A-N, the food safety regulations 1038A-N, the facility data 1040, and/or the cooling environment operational data 1042A-N. Refer to at least FIGS. 8A, 8B, 9A, and 9B for further discussion about generating and/or modifying the cooling cycles.

The item routing determiner 1016 can be configured to generate and/or modify instructions for routing the items throughout the facility 1068, to one or more controlled environments 100, and/or to one or more storage locations in the facility 1068. The determiner 1016 can use any determinations made by the item condition determiner 1010, the environmental control engine 1012, and/or the blast scheduling engine 1014 to generate and/or modify routing instructions for the items received in the facility 1068. In some implementations, the determiner 1016 can generate and/or modify the routing instructions using one or more data retrieved/accessed from the data store 1002, such as the item data 1034, the existing cooling environment schedules 1044A-N, the cooling criteria 1036A-N, the food safety regulations 1038A-N, the facility data 1040, and/or the cooling environment operational data 1042A-N. Refer to at least FIGS. 8A, 8B, 9A, and 9B for further discussion about generating and/or modifying the item routing instructions.

The optional item grouping determiner 1018 can be configured to generate and/or modify grouping information for items received in the facility 1068 that can be loaded into the controlled environment 100 together, cooled/frozen under similar and/or same conditions in the controlled environment 100, and/or routed to one or more storage locations in the facility 1068 together. The optional determiner 1018 can use any determinations made by the item condition determiner 1010, the environmental control engine 1012, the blast scheduling engine 1014, and/or the item routing determiner 1016 to generate and/or modify grouping of items. In some implementations, the engine 1014 can generate and/or modify the groupings using one or more data retrieved/accessed from the data store 1002, such as the item data 1034, the existing cooling environment schedules 1044A-N, the cooling criteria 1036A-N, the food safety regulations 1038A-N, the facility data 1040, and/or the cooling environment operational data 1042A-N. Refer to at least FIGS. 8A, 8B, 9A, and 9B for further discussion about generating and/or modifying the item groupings.

The blast flywheeling determiner 1020 can be configured to generate and/or modify operational instructions for one or more of the controlled environments 100 in the facility 1068. The operational instructions can be generated in order to optimize scheduling of tasks and completion of operations in the facility 1068 while also efficiently utilizing energy resources. Thus, the determiner 1020 can determine when to operate one or more of the controlled environments 100 in light of energy supply, energy demand, and/or energy prices, as well as food safety regulations and standards corresponding to items that require to be cooled/frozen in the controlled environments 100.

The determiner 1020 can use any determinations made by the item condition determiner 1010, the environmental control engine 1012, the blast scheduling engine 1014, the item routing determiner 1016, and/or the optional item grouping determiner 1018 to generate and/or modify blast flywheeling instructions. The determiner 1020 can also receive sensor data from the environmental sensors 130A-N and/or the sensor device 106, which can be used to generate and/or modify the blast flyhweeling instructions. In some implementations, the determiner 1020 can generate and/or modify the instructions using one or more data retrieved/accessed from the data store 1002, such as the item data 1034, the existing cooling environment schedules 1044A-N, the cooling criteria 1036A-N, the food safety regulations 1038A-N, the facility data 1040, and/or the cooling environment operational data 1042A-N. The determiner 1020 can also generate and/or modify the instructions based on receiving information from other computing systems, such as an energy market computing system and/or an energy provider system. Refer to at least FIGS. 8A, 8B, 9A, and 9B for further discussion about generating and/or modifying the blast flywheeling instructions.

The output generator 1022 can be configured to generate one or more graphical user interfaces (GUIs) for presenting information at the user device(s) 1000. For example, the generator 1022 can receive any of the determined information from other components of the control system 116 and generate GUIs to present that information. The generator 1022 can also generate GUIs that present any of the information that is generated by other components described herein, such as the sensor device 106, the environmental sensors 130A-N, and/or the environmental control unit 118. The generator 1022 can then transmit the output to the user device(s) 1000.

The sensor device 106 can include the integrated circuit 200 and hardware or physical components such as the adhesive(s) 208, the vent(s) 206A-N, and the housing 204. The integrated circuit 200 can include the wireless transceiver 112, the temperature sensor(s) 108, the other sensors 202A-N, the acoustic sensor(s) 110, the power source 114, an optional processor(s) 1046, and/or the ultrasonic transducers 203A-N. The processor(s) 1046 can be configured to perform operations or otherwise execute instructions that cause one or more of the other components of the sensor device 106 to perform the operations. As described herein, the sensor device 106 can be designed to ensure battery/power source life can be sustained for extended periods of time in inclement conditions (e.g., cold or freezing temperatures) so that the sensors of the sensor device 106 can continue to measure signals and transmit signals in real-time or near real-time to the control system 116. Refer to FIGS. 1A, 1B, and 2 for further discussion about the components of the sensor device 106.

The user device(s) 1000 can be any type of computing device such as a laptop, tablet, computer, mobile phone, smart phone, and/or wearable device. The user device 1000 can be used by relevant users associated with the facility 1068, including but not limited to facility workers, operational managers or workers in the facility, customers of the items being stored in the facility, or other relevant stakeholders. The user device(s) 1000 can include input devices (e.g., keyboards, touch screens, mice, microphones) and output devices (e.g., display screens, speakers, haptic feedback) for receiving user input and presenting information or output to the user. As described herein, the user device(s) 1000 can output GUIs that are generated by the output generator 1022. The user device(s) 1000 can receive user input indicating one or more changes or modifications to a cooling cycle/blast schedule, item routing, item grouping, blast flywheeling, and/or environment control(s) that can be generated by components of the control system 116. The user input can then be transmitted to the control system 116 and used to adjust the controlled environment 100 or facility operations accordingly.

The user device(s) 1000 can additionally or alternatively present GUIs with information about energy markets, facility energy usage, and/or operations/scheduling tasks in the facility 1068. The relevant user can view this information to make determinations and/or adjustments to any of the control operations and/or scheduling tasks described herein. For example, the user can provide user input indicating a desire to flywheel cooling cycles in one or more of the controlled environments 100 at a particular time. The user can provide user input indicating a desire to change schedules and/or movements of the one or more items and/or groups of items in the facility 1068. The user input can be received by at least the control system 116 and executed by components in the facility 1068, as described herein.

The sensor device(s) 1302 can be configured to detect conditions surrounding items a facility or in the items themselves, such as ultrasonic wave signals, temperature signals, humidity signals, heat flux signals, and/or transfer of heat coefficient signals. Signals collected by the sensor device(s) 1302 can be transmitted to the control system 116 and used as inputs to one or more of the models described herein to determine conditions of the items (e.g., temperature, phase state). Refer to FIGS. 13A and 13B for further discussion about the sensor device(s) 1302.

Still referring to FIGS. 10A and 10B, a plurality of the controlled environments 100 can be located in the facility 1068. The facility 1068 can be any type of storage facility, cold storage, warehouse, and/or distribution center. The controlled environments 100 can be blast cells, as described herein. Each of the controlled environments 100 may include the environmental control unit 118, the cooling system 132A-N, and/or the environmental sensors 130A-N. In some implementations, one or more of the components 118, 132A-N, and/or 130A-N can be remote from the controlled environment(s) 100 but communicably coupled (e.g., wired, wirelessly via network(s)) to components inside the controlled environment(s) 100.

The environmental sensors 130A-N, for example, can be positioned throughout the facility 1068 and the controlled environments 100. One or more environmental sensors 130A-N can be positioned outside or near the controlled environments 100, such as around vents or other components for providing supply air to the controlled environments 100 and receiving return air from the controlled environments 100. The environmental sensors 130A-N, as described herein, can include temperature, acoustic, ambient air, motion, air flow, and/or humidity sensors. One or more other sensors are also possible.

The environmental control unit 118 can include a communication interface 1048, processor(s) 1050, a controller 1052, and an optional controls determiner 1054. The control unit 118 can be configured to generate and/or execute operations for controlling components associated with the respective controlled environment 100. The control unit 118 can control components of the cooling system 132A-N corresponding to the respective controlled environment 100, for example.

The communication interface 1048 can provide communication between components of the environmental control unit 118 and other system components described in FIGS. 10A and 10B. The processor(s) 1050 can be configured to perform or execute instructions for performing the operations described herein.

The controller 1052 and be configured to execute controls, adjustments to controls, operations, and/or other tasks to control the cooling system 132A-N. The controller 1052 can receive control instructions from the environmental control engine 1012, the blast scheduling engine 1014, the blast flywheeling determiner 1020, and/or the user device(s) 1000. The controller 1052 can then execute the received control instructions. In some implementations, the controller 1052 can access or retrieve the cooling environment schedules 1044A-N and/or the cooling environment operational data 1042A-N from the data store 1002 to then execute the control instructions for the particular controlled environment 100.

The optional controls determiner 1054 can be configured to generate one or more controls for the cooling system 132A-N. Refer to the environmental control engine 1012 of the control system 116 for further discussion.

Each of the cooling systems 132A-N can be configured to operate cooling cycles to lower a temperature inside a respective controlled environment 100. The cooling systems 132A-N can therefore be configured to lower temperatures of items placed inside the controlled environments 100 to temperatures that correspond to desired cooled or frozen temperatures for the items. Each of the cooling systems 132A-N may include a communication interface 1056, a controller 1058, fans 1064A-N, a refrigeration system 1062, an optional controls determiner 1060, and sensors 1066A-N.

The communication interface 1056 can be configured to provide communication between components of the cooling system 132A-N and other system components described in FIGS. 10A and 10B.

The controller 1058 can be configured to execute instructions to control components of the cooling system 132A-N. The instructions can be received from the environmental control unit 118, the environmental control engine 1012, the blast scheduling engine 1014, the blast flywheeling determiner 1020, and/or the user device(s) 1000. Sometimes, the controller 1058 can retrieve control instructions from the data store 1002, such as the cooling environment schedules 1044A-N and/or the cooling environment operational data 1042A-N.

The fans 1064A-N can be configured to circulate supply air and return air inside the controlled environment 100. For example, the fans 1064A-N can be positioned at various locations in the controlled environment 100 to push or otherwise move the supply air from the refrigeration system 1062 around pallets of items that are arranged inside the controlled environment 100. In some implementations, one or more of the fans 1064A-N can be positioned near vents or channels for supply air entering the controlled environment 100. One or more of the fans 1064A-N can also be positioned near vents and/or channels for routing return air out of the controlled environment 100.

The refrigeration system 1062 can be configured to provide cool supply air to the controlled environment 100 to cool/freeze the items placed therein. The refrigeration system 1062 can include an HVAC unit or any other type of components that can be used to cool or otherwise freeze items placed therein. The refrigeration system 1062 can be controlled by the controller 1058.

The optional controls determiner 1060 can be configured to generate one or more controls for the components of the cooling system 132A-N. Refer to the environmental control engine 1012 of the control system 116 for further discussion.

The sensors 1066A-N can be any type of sensors described herein. The sensors 1066A-N can be the same as or similar to the temperature sensor(s) 108, the acoustic sensor(s) 110, the other sensor(s) 202A-N, and/or the environmental sensors 130A-N.

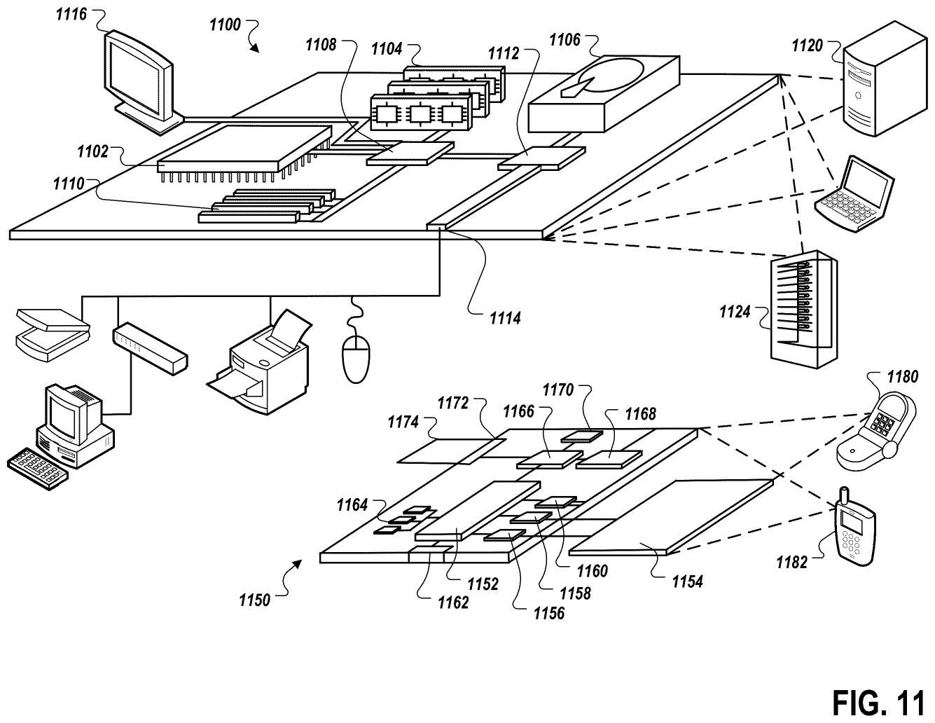

FIG. 11 shows an example of a computing device 1100 and an example of a mobile computing device that can be used to implement the techniques described here. The computing device 1100 is intended to represent various forms of digital computers, such as laptops, desktops, workstations, personal digital assistants, servers, blade servers, mainframes, and other appropriate computers. The mobile computing device is intended to represent various forms of mobile devices, such as personal digital assistants, cellular telephones, smart-phones, and other similar computing devices. The components shown here, their connections and relationships, and their functions, are meant to be exemplary only, and are not meant to limit implementations of the inventions described and/or claimed in this document.

The computing device 1100 includes a processor 1102, a memory 1104, a storage device 1106, a high-speed interface 1108 connecting to the memory 1104 and multiple high-speed expansion ports 1110, and a low-speed interface 1112 connecting to a low-speed expansion port 1114 and the storage device 1106. Each of the processor 1102, the memory 1104, the storage device 1106, the high-speed interface 1108, the high-speed expansion ports 1110, and the low-speed interface 1112, are interconnected using various busses, and can be mounted on a common motherboard or in other manners as appropriate. The processor 1102 can process instructions for execution within the computing device 1100, including instructions stored in the memory 1104 or on the storage device 1106 to display graphical information for a GUI on an external input/output device, such as a display 1116 coupled to the high-speed interface 1108. In other implementations, multiple processors and/or multiple buses can be used, as appropriate, along with multiple memories and types of memory. Also, multiple computing devices can be connected, with each device providing portions of the necessary operations (e.g., as a server bank, a group of blade servers, or a multi-processor system).

The memory 1104 stores information within the computing device 1100. In some implementations, the memory 1104 is a volatile memory unit or units. In some implementations, the memory 1104 is a non-volatile memory unit or units. The memory 1104 can also be another form of computer-readable medium, such as a magnetic or optical disk.

The storage device 1106 is capable of providing mass storage for the computing device 1100. In some implementations, the storage device 1106 can be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product can also contain instructions that, when executed, perform one or more methods, such as those described above. The computer program product can also be tangibly embodied in a computer- or machine-readable medium, such as the memory 1104, the storage device 1106, or memory on the processor 1102.

The high-speed interface 1108 manages bandwidth-intensive operations for the computing device 1100, while the low-speed interface 1112 manages lower bandwidth-intensive operations. Such allocation of functions is exemplary only. In some implementations, the high-speed interface 1108 is coupled to the memory 1104, the display 1116 (e.g., through a graphics processor or accelerator), and to the high-speed expansion ports 1110, which can accept various expansion cards (not shown). In the implementation, the low-speed interface 1112 is coupled to the storage device 1106 and the low-speed expansion port 1114. The low-speed expansion port 1114, which can include various communication ports (e.g., USB, Bluetooth, Ethernet, wireless Ethernet) can be coupled to one or more input/output devices, such as a keyboard, a pointing device, a scanner, or a networking device such as a switch or router, e.g., through a network adapter.

The computing device 1100 can be implemented in a number of different forms, as shown in the figure. For example, it can be implemented as a standard server 1120, or multiple times in a group of such servers. In addition, it can be implemented in a personal computer such as a laptop computer 1122. It can also be implemented as part of a rack server system 1124. Alternatively, components from the computing device 1100 can be combined with other components in a mobile device (not shown), such as a mobile computing device 1150. Each of such devices can contain one or more of the computing device 1100 and the mobile computing device 1150, and an entire system can be made up of multiple computing devices communicating with each other.

The mobile computing device 1150 includes a processor 1152, a memory 1164, an input/output device such as a display 1154, a communication interface 1166, and a transceiver 1168, among other components. The mobile computing device 1150 can also be provided with a storage device, such as a micro-drive or other device, to provide additional storage. Each of the processor 1152, the memory 1164, the display 1154, the communication interface 1166, and the transceiver 1168, are interconnected using various buses, and several of the components can be mounted on a common motherboard or in other manners as appropriate.

The processor 1152 can execute instructions within the mobile computing device 1150, including instructions stored in the memory 1164. The processor 1152 can be implemented as a chipset of chips that include separate and multiple analog and digital processors. The processor 1152 can provide, for example, for coordination of the other components of the mobile computing device 1150, such as control of user interfaces, applications run by the mobile computing device 1150, and wireless communication by the mobile computing device 1150.

The processor 1152 can communicate with a user through a control interface 1158 and a display interface 1156 coupled to the display 1154. The display 1154 can be, for example, a TFT (Thin-Film-Transistor Liquid Crystal Display) display or an OLED (Organic Light Emitting Diode) display, or other appropriate display technology. The display interface 1156 can comprise appropriate circuitry for driving the display 1154 to present graphical and other information to a user. The control interface 1158 can receive commands from a user and convert them for submission to the processor 1152. In addition, an external interface 1162 can provide communication with the processor 1152, so as to enable near area communication of the mobile computing device 1150 with other devices. The external interface 1162 can provide, for example, for wired communication in some implementations, or for wireless communication in other implementations, and multiple interfaces can also be used.

The memory 1164 stores information within the mobile computing device 1150. The memory 1164 can be implemented as one or more of a computer-readable medium or media, a volatile memory unit or units, or a non-volatile memory unit or units. An expansion memory 1174 can also be provided and connected to the mobile computing device 1150 through an expansion interface 1172, which can include, for example, a SIMM (Single In Line Memory Module) card interface. The expansion memory 1174 can provide extra storage space for the mobile computing device 1150, or can also store applications or other information for the mobile computing device 1150. Specifically, the expansion memory 1174 can include instructions to carry out or supplement the processes described above, and can include secure information also. Thus, for example, the expansion memory 1174 can be provide as a security module for the mobile computing device 1150, and can be programmed with instructions that permit secure use of the mobile computing device 1150. In addition, secure applications can be provided via the SIMM cards, along with additional information, such as placing identifying information on the SIMM card in a non-hackable manner.

The memory can include, for example, flash memory and/or NVRAM memory (non-volatile random access memory), as discussed below. In some implementations, a computer program product is tangibly embodied in an information carrier. The computer program product contains instructions that, when executed, perform one or more methods, such as those described above. The computer program product can be a computer- or machine-readable medium, such as the memory 1164, the expansion memory 1174, or memory on the processor 1152. In some implementations, the computer program product can be received in a propagated signal, for example, over the transceiver 1168 or the external interface 1162.

The mobile computing device 1150 can communicate wirelessly through the communication interface 1166, which can include digital signal processing circuitry where necessary. The communication interface 1166 can provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication can occur, for example, through the transceiver 1168 using a radio-frequency. In addition, short-range communication can occur, such as using a Bluetooth, WiFi, or other such transceiver (not shown). In addition, a GPS (Global Positioning System) receiver module 1170 can provide additional navigation- and location-related wireless data to the mobile computing device 1150, which can be used as appropriate by applications running on the mobile computing device 1150.

The mobile computing device 1150 can also communicate audibly using an audio codec 1160, which can receive spoken information from a user and convert it to usable digital information. The audio codec 1160 can likewise generate audible sound for a user, such as through a speaker, e.g., in a handset of the mobile computing device 1150. Such sound can include sound from voice telephone calls, can include recorded sound (e.g., voice messages, music files, etc.) and can also include sound generated by applications operating on the mobile computing device 1150.

The mobile computing device 1150 can be implemented in a number of different forms, as shown in the figure. For example, it can be implemented as a cellular telephone 1180. It can also be implemented as part of a smart-phone 1182, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

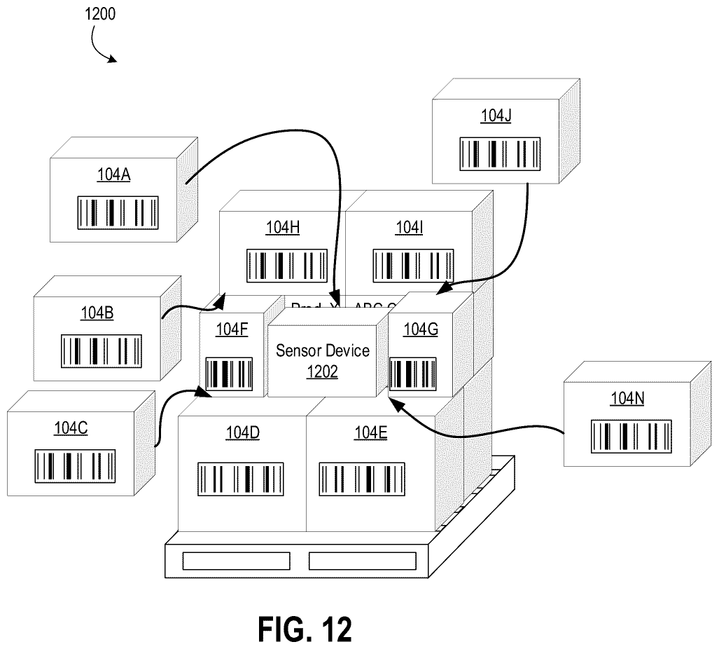

FIG. 12 illustrates an example pallet 1200 having a sensor device 1202 positioned between cases 104A-N of items on the pallet 1200. The sensor device 1202 can be the same as or similar to the sensor device 106 and/or any of the implementations of the sensor device described herein. The sensor device 1202 can be an empty case having similar properties (e.g., dimensions, size) as any of the cases 104A-N on the pallet 1200. When the pallet 1200 is assembled with the cases 104A-N to then be put inside a controlled environment described herein, a worker, forklift, or autonomous vehicle can arrange the cases 104A-N on the pallet 1200 with the sensor device 1202 positioned at a center of the pallet 1200 (e.g., equidistant from each outside side of the stacked cases 104A-N and halfway up a total height of the cases 104A-N once stacked on the pallet 1200. The sensor device 1202 can be shaped and designed as a stand-in for any of the cases 104A-N. The sensor device 1202 can include a plurality of different types of sensors, including but not limited to temperature, acoustic, humidity, and other types of sensors. The sensor device 1202 can accordingly collect data about cases that are adjacent to the sensor device 1202 on the pallet 1200. More data can be collected about the plurality of cases 104A-N to accurately determine internal conditions of the items inside the cases 104A-N in real-time or near real-time.

FIG. 13A is a conceptual diagram of a system 1300 for determining a phase state of items in a facility based on modeling ultrasonic wave signals. The pallet 102, as described herein, can include the cases 104A-N. The pallet 102 can be placed in a controlled environment, such as a blast cell or freezing/cooling room in the facility. In some implementations, the cases 104A-N can be disassembled from the pallet 102 and positioned/placed in the controlled environment. In the example of FIG. 13A, a sensor device 1302 can be positioned amongst the cases 104A-N on the pallet 102. In some implementations, as shown in FIG. 13B, the sensor device 1302 can be configured to attach or otherwise affix to one or more of the cases 104A-N. As an illustrative example, for every given product/item type in a blast cell, at least one of those products/items can be configured with or proximate/adjacent a sensor device 1302. In some implementations, the sensor device(s) 1302 can be attached/affixed to or otherwise placed proximate one or more of the cases 104A-N that are positioned in least desirable locations in the blast cell. The least desirable locations in the blast cell can include locations where the one or more cases 104A-N receive a less than optimal flow of air to freeze/cool the items therein. For example, the least desirable location in the blast cell may include a top row of the cases 104A-N, closest fans and/or a refrigeration system in the blast cell. Thus, in blast cells where air is moved from the top down, the cases 104A-N in the top row may receive the least amount of air and/or may receive the air after it has circulated down, into, and through other rows in the blast cell. In some implementations, the least desirable locations in the blast cell can vary based on a setup/configuration of the blast cell and/or a direction of airflow in the blast cell. In yet some implementations, the cases 104A-N in dense, middle rows may be identified as the least desirable locations and/or the cases 104A-N that are farthest away from the fans and/or the refrigeration system can be considered in the least desirable locations.

The sensor device 1302 can be configured to generate and receive one or more signals indicating ambient conditions of the cases 104A-N of items on the pallet 102. The signals can include ultrasonic wave signals, temperature signals, or other signals described herein. The signals can be transmitted over the network(s) 120 to a computer system 1320 for additional processing to determine conditions of the items in the cases 104A-N, such as a phase state of the items and/or temperature conditions of the items. The computer system 1320 can be any type of computing system described herein, configured to process the signals received from the sensor device 1302 to determine conditions of the items in the cases 104A-N. The computer system 1320 can also be configured to train one or more machine learning models for runtime use in determining the conditions of the items in the cases 104A-N. Refer to FIGS. 15A, 15B, 15C, and 16 for further discussion about operations performed by the computer system 1320. In some implementations, the computer system 1320 may be part of or the same as the control system 116 described herein.

The sensor device 1302 may include ultrasonic transducers 1304A-N, processor(s) 1306, signal convertor 1308, signal generator 1310, wireless transceiver 1312, optional temperature sensor(s) 108, and/or a power source 1314. The processor(s) 1306 can be configured to store signals and/or control the ultrasonic transducers 1304A-N. The wireless transceiver 1312 can be configured to transmit the signals collected at the sensor device 1302 to the computer system 1320 over the network(s) 120. In some implementations, the transducers 1304A-N can be attached to the case 104A using one or more adhesive features, such as straps or ratchet straps. Attaching the transducers 1304A-N like so can ensure that the transducers 1304A-N and/or other components, such as the temperature sensor(s) 108 do not move or shift during a cycle of emitting and receiving soundwaves.

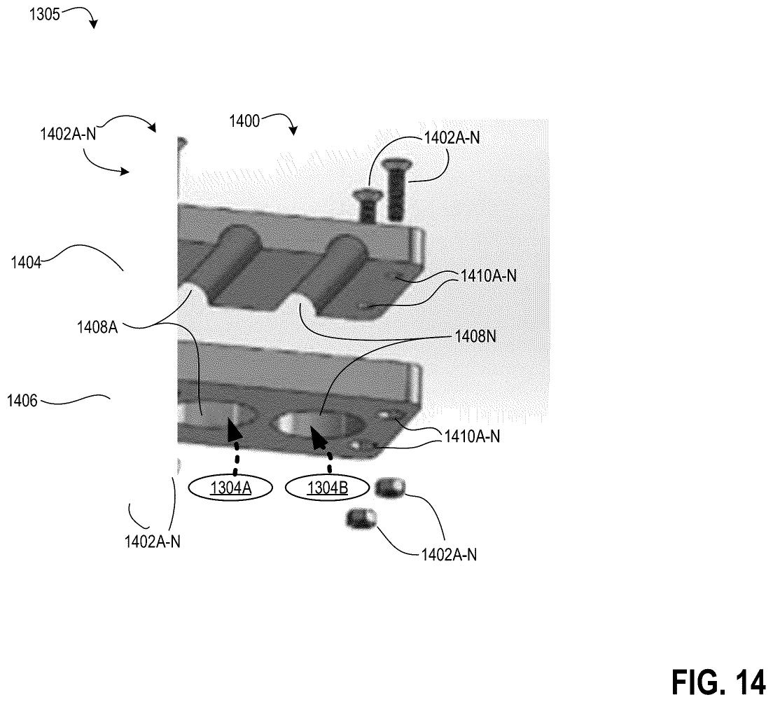

In some implementations, the sensor device 1302 may include two ultrasonic transducers 1304A-N-one to emit ultrasonic soundwaves and another to receive the ultrasonic soundwaves. Sometimes, as shown in FIG. 14, the ultrasonic transducers 1304A-N can be physically arranged on a same side of the sensor device 1302. The signal generator 1310 can be configured to generate ultrasonic wave pulses to be sent by one or more of the ultrasonic transducers 1304A-N. Sometimes, the signal generator 1310 can be a pulse moderator. The signal convertor 1308 can be configured to read the received signals from one or more of the ultrasonic transducers 1304A-N and convert the signals to a format that is readable by the computer system 1320. For example, the signal convertor 1308 can be an analog-to-digital convertor (ADC).

Still referring to the system 1300 in FIG. 13A, the sensor device 1302 can generate sensor data in block A (1322). The sensor data, as described herein, can include ultrasonic wave signals and/or temperature signals. Generating the sensor data can include the processor(s) 1306 of the sensor device 1302 executing instructions to cause: the signal generator 1310 to generate ultrasonic waves/pulses, the ultrasonic transducers 1304A-N to emit and receive those ultrasonic waves/pulses, the signal convertor 1308 to convert the received signals to a computer-readable format, and the wireless transceiver 1312 to transmit the converted signals as the sensor data to the computer system 1320.

Accordingly, once the sensor data is generated, the sensor data can be transmitted to the computer system 1320 over the network(s) 120 (block B, 1324).

The computer system 1320 can apply one or more machine learning models to the data in block C (1326). The machine learning models, as described herein, can include but are not limited to NNs and/or CNNs. The models can be trained to determine conditions of the items in the cases 104A-N, such as temperature and/or phase states of the items. The models can be trained to generate probabilities or likelihoods of the phase states of the items, as described further in reference to FIGS. 15A, 15B, 15C, and 16.

Each material phase can have characteristic signals. The models can be trained to identify and correlate such characteristic signals with different phases, such as liquid, solid, and/or mixed (liquid and solid, such as a phase change) phases. For example, the liquid phase can have a characteristic signal of 1 ultrasonic wave reflection. The solid phase may also have a characteristic signal of 1 ultrasonic wave reflection. The mixed phase may have a characteristic signal of multiple ultrasonic wave reflections. Moreover, the models can also be trained to detect and correlate the signals with the different phases and different materials or packaging of the items in the cases 104A-N. The different materials and/or packaging may impact, for example, how slowly or how quickly the items may cool to reach a desired freezing/cooling temperature. Similarly, different types of items may also impact how slowly or quickly the items cool. Liquid products, such as concentrates and/or juices, may not develop air gaps, which means that the packaging or material of the cases 104A-N for such items may not be a prevalent parameter or factor in determining the conditions of the liquid products. On the other hand, gel-based products (e.g., jams, jellies) and/or solid products (e.g., peas, corn, chicken) may develop air gaps in their cases 104A-N, which can result in more ultrasonic wave reflections and, subsequently, additional signal processing techniques. The models can therefore be trained with additional parameters, such as distribution of the products inside the cases 104A-N, packaging, case materials, types of products/items, etc.

Once the model(s) is applied to the received data, the computer system 1320 can determine phase information for the items in the cases 104A-N(block D, 1328). As described in reference to FIGS. 15A, 15B, and 15C, the computer system 1320 can apply one or more filtering techniques, thresholds, and/or criteria to the model output to determine a current, expected, and/or projected phase state of the items in the cases 104A-N.

The computer system 1320 may return the phase information in block E (1330). The phase information can be used by relevant users in the facility. For example, the phase information can be used to determine whether cooling operations/blast cycles are properly determined/performed or whether they should be adjusted (e.g., increase duration of running a cycle, decrease the duration). The phase information can be used to determine whether any of the cases 104A-N should be removed from the blast cell or other controlled environment. The phase information can be used to determine whether any of the cases 104A-N should be put into a blast cell or other controlled environment to reach a desired temperature for storage and/or transportation. In some implementations, the phase information can be used to determine whether refrigeration systems in trucks or other transport vehicles are operating as expected or desired while the cases 104A-N are in transit to consumers, producers, end users, facilities, and/or warehouses. Any of the determinations described in reference to block E (1330) can be performed automatically by the computer system 1320 or other computing systems described herein. In some implementations, one or more of the determinations can be additionally or alternatively performed by the relevant users.

FIG. 13B is a conceptual diagram of a sensor device 1305 configured to attach to an example case 104A of items to generate and receive ultrasonic wave signals. The sensor device 1305 can be similar to the sensor device 1302 described herein. For example, the sensor device 1305 can include one or more of the components described in reference to the sensor device 1302 in FIG. 13A. Here, the sensor device 1305 can be configured to attach or otherwise affix to an individual case, such as the case 104A. The sensor device 1305 can include at least the ultrasonic transducers 1304A and 1304B, which can be positioned alongside each other or otherwise parallel to each other within the sensor device 1305. Refer to FIG. 14 for further discussion about the configuration of the ultrasonic transducers 1304A and 1304B.

The sensor device 1305 can be easily attached to or detached from different cases. For example, the sensor device 1305 can be attached to a case using one or more types of bands, tapes, adhesives, elastics, and/or straps. As an illustrative example, the sensor device 1305 can be easily and efficiently attached to a case using ratchet straps, thereby allowing the sensor device 1305 to be easily detached from the case and applied to other cases. Therefore, the disclosed configuration of the sensor device 1305 allows for the sensor device 1305 to be quickly and efficiently deployed in different facilities and/or other settings. The sensor device 1305 can be affixed to the case 104A, can measure ultrasonic wave signals for the case 104A, and then be removed from the case 104A and attached to another case to measure the signals for the other case.

FIG. 14 illustrates the sensor device 1305 of FIG. 13B. As described herein, the sensor device 1305 can be the same as or similar to the sensor device 1302. The sensor device 1302 may include one or more of the components described in FIG. 14 in reference to the sensor device 1305.

The sensor device 1305 can include a case 1400 (e.g., holder), which can be configured to house components described herein. The components can include at least the ultrasonic transducers 1304A and 1304B. The case 1400 can also be configured to house other components described in reference to FIG. 13A, including but not limited to the processor(s) 1306, the signal convertor 1308, the signal generator 1310, the wireless transceiver 1312, the temperature sensor(s) 108, and/or the power source 1314. For illustrative purposes, the case 1400 is shown as only housing the ultrasonic transducers 1304A and 1304B in FIG. 14.

The case 1400 may include an upper portion 1404 and a lower portion 1406, which can be configured to affix to each other to form the case 1400 (e.g., the housing). When affixed to each other, the upper portion 1404 and the lower portion 1406 can form defined openings 1408A-N through which to receive and house the components of the sensor device 1305. For example, the openings 1408A-N can be shaped, sized, and defined to hold, house, or otherwise retain the ultrasonic transducers 1304A and 1304B. The upper portion 1404 and the lower portion 1406 of the case 1400 can be affixed to each other using fastening elements 1402A-N. The fastening elements 1402A-N can be fitted into and through openings 1410A-N in the upper portion 1404 and the lower portion 1406 of the case 1400. In some implementations, the fastening elements 1402A-N may include screws, nuts, and/or bolts.

The construction and configuration of the case 1400 can make it easy and efficient to service and/or build the sensor device 1305 in any setting. For example, if a component such as the ultrasonic transducer 1304A needs servicing and/or replacement, any relevant user can simply and easily unscrew the fastening elements 1402A-N, remove the fastening elements 1402A-N from the openings 1410A-N, disassemble the upper portion 1404 and the lower portion 1406, and service the ultrasonic transducer 1304A. The user may then simply and easily reassemble the components of the case 1400 before deploying the sensor device 1305 back in the field.

FIGS. 15A, 15B, and 15C are a flowchart of a process 1500 for determining a phase state of items based on modeling ultrasonic wave signals. Although the process 1500 is described from the perspective of determining the phase state of items in a case or a pallet, the process 1500 can be performed for many different items, many different cases, and/or many different pallets. Sometimes, the process 1500 can be performed in parallel for the different cases and/or the different pallets.

The process 1500 can be performed by components described herein, such as the computer system 1320. In some implementations, the process 1500 can be performed by the control system 116 or any other computing systems/devices described. For illustrative purposes, the process 1500 is described from the perspective of a computer system.

Referring to the process 1500 in FIGS. 15A, 5B, and 15C, the computer system can receive data signals for a case or pallet of items in block 1502. The computer system may receive ultrasonic wave signals and/or reflections at predetermined time intervals (block 1504). For example, a sensor device as described herein can be configured to measure reflections of ultrasonic waves that are emitted by the sensor device at the predetermined time intervals (e.g., every n minutes). The computer system may receive temperature signals (block 1506). As described herein, the sensor device can include one or more temperature sensors configured to detect ambient temperature conditions surrounding, at, or otherwise near the case/pallet of items. The computer system may receive humidity signals (block 1508), similarly to the temperature signals. The computer system may optionally receive heat flux data (block 1510). The heat flux data can be generated by the sensor device described herein and can measure a rate of heat transfer per unit area. The sensor device can, for example, include a heat flux sensor having thermocouples and/or thermopiles to measure a temperature difference across a surface (e.g., the surface of the case/pallet of items). Knowing thermal properties of the material making up the surface, the heat flux can be determined. The computer system may optionally receive heat transfer coefficient data (block 1512). The heat transfer coefficient data can indicate a measure of thermal conductivity or ability of the material of the case/pallet to conduct heat. The heat transfer coefficient data can be determined by the computer system or another computing system using signals that are generated by the sensors described herein. For example, the computer system can determine the heat transfer coefficient for the case/pallet of items based on applying one or more models and/or empirical correlations to the temperature signals and/or the heat flux data. The computer system may use a physics-based model to mine the heat transfer coefficient data, which can be used as an additional input to other models described herein.

Any of the data signals that are received in blocks 1502-1512 can be generated by sensor devices described herein at the predetermined time intervals. For example, the data signals can be generated every 5 minutes, every 1 minute, every 10 minutes, every 15 minutes, every 1 hour, etc. The predetermined time intervals can vary based on parameters, including but not limited to a type of location having the case or pallet of items (e.g., blast freezer, blast cell, cold-storage room, external environment, freight or other transport vehicle), a type of the items on the case/pallet, packaging used for the case/pallet, materials used for the case/pallet, etc. Refer to at least FIGS. 1A, 1B, 2A, 2B, 13A, and 13B for further discussion about generating and transmitting the various sensor signals described herein.

The computer system can process the data signals based on applying filtering techniques in block 1514. For example, the computer system may apply a high pass filter to one or more of the data signals in block 1516. Sometimes, the computer system may apply a band pass filter. Sometimes, the computer system can additionally or alternatively filter the data signals using parameters such as amplitude of the data signals (e.g., amplitude of a soundwave). The high pass filter can be applied to some or all of the received signals to reduce noise (e.g., low-frequency noise) that may be present in the signals (e.g., in acoustic signals, in ultrasonic wave signals). The high pass filter can have a determined cut off frequency. An illustrative cut off frequency can include 6.6 kHz. One or more greater or lesser cut off frequencies may be defined and used with the disclosed techniques.

Optionally, the computer system may apply filtering techniques based on packaging and/or materials of the cases/pallets of items (block 1518). Sometimes, for example, the computer system may apply one or more machine learning models that are trained to process the received signals based on packaging/material parameters. Certain packaging and/or materials may make it more challenging for cold air to permeate through to cool an internal temperature of the items in the case/pallet. The data signals collected from the case/pallet having such packaging and/or materials can be filtered differently than cases/pallets having other packaging and/or materials to ensure sufficient sensitivity to the strength of signals being generated and collected.

The computer system can retrieve one or more trained models in block 1520. The models can include an ultrasonic phase model (block 1522). Refer to FIG. 16 for further discussion about training the ultrasonic phase model. Additionally or alternatively, the computer system may optionally retrieve a temperature phase model (block 1524), a phase model (block 1526), and/or a physics-based model (block 1528). The computer system can use any combination of models described herein to generate information about the items in the case/pallet. Such information can include internal temperature information and/or phase state information. Any of the models described herein can be NNs and/or CNNs. Advantageously, NNs and/or CNNs can be complex enough models that they can analyze various signals and signal types, and identify differences between the signals that may correspond to different phases (e.g., solid, liquid, mixed). The model(s) can include, as an illustrative example, 2 convolutional layers and 3 normal layers. The models can be trained using the techniques described throughout this document.

In some implementations, the computer system can process all of the received signals through a single model, such as the phase model (block 1526), to determine the information about the items. As another example, the computer system can process the ultrasonic wave signals through the ultrasonic phase model (block 1522) and the temperature signals through the temperature phase model (block 1524). Then, the computer system can combine outputs from both models and apply one or more rules and/or criteria to the combined output to determine the information about the items. As yet another example, the computer system can process the outputs from both models through another model, such as the phase model (block 1526). As another example, the computer system can use the physics-based model (block 1528) to mine heat transfer coefficient information, which can then be fed as additional inputs to one or more of the other models described herein.

In block 1530, the computer system can provide the processed data signals as inputs to the trained model(s). As an illustrative example, the computer system can provide the processed ultrasonic wave signals as input to the ultrasonic phase model (block 1522).

The computer system can receive, from the trained model(s), model output indicating likelihood of phase states of the items in the cases/pallets (block 1532). A NN, for example, can include a softmax function in an output layer of the NN, which can be used to make the output represent probabilities across multiple classes. For example, the model output can include a value (e.g., numeric, integer, percentage, probability, ratio) corresponding to a likelihood that the items are in a mixed phase (block 1534). The model output can include a value corresponding to a likelihood that the items are in a solid phase (block 1536). The model output can include a value corresponding to a likelihood that the items are in a liquid phase (block 1538). In some implementations, the model(s) can be trained to generate output including all three of the values in blocks 1534-1538. In some implementations, the model(s) can be trained to generate output including less than all three of the values, such as one of the values, in blocks 1534-1538.

Next, the computer system may reduce noise in the model outputs based on performing a moving average of the received data signals (block 1540). Performing the moving average can help with smoothing out fluctuations or noise in a time series or sequential/consecutive data as described herein. By taking an average over a window of consecutive data points, the overall trend can become more apparent while reducing the influence of short-term fluctuations in the data. Removing the short-term fluctuations may also help with identifying trends or patterns, such as when the items have reached a different phase/phase change during a cooling cycle.

In some implementations, the moving average techniques may include recursive moving averages and/or rolling moving averages. Both techniques can be used to smooth data and identify trends. In a recursive moving average, a current moving average value can be calculated based on a previous moving average value and the addition of a new data point. The moving average can be updated as new data becomes available without recalculating the entire series. This approach can be advantageously computationally efficient since it may not require storing an entire data series. In the rolling moving average, a fixed-size window (e.g., 12 1 hour windows, 2 12 hour windows, 5 1 hour windows) can be applied to the data, and the average can be calculated within that window as it moves through the data series. As new data points become available, that data can enter the window, and old data points may exit. Rolling moving averages can be useful for smoothing data and identifying trends over a fixed period. They can also be less sensitive to outliers compared to the recursive moving average.

For example, the computer system can identify a time interval to compute the moving average (block 1542). As an illustrative example, the time interval can be 5 minute blocks. One or more other time intervals are possible, including but not limited to every 1 minute, every 2 minutes, every 10 minutes, every 15 minutes, etc. The time interval can correspond to the predetermined time intervals at which the data signals are generated/collected in blocks 1502-1512. The computer system can, for each data point in the received data signals for the identified time interval, compute an average of data points within the time interval centered at the data point (block 1544). The computer system can also compare the received data signals to the computed moving averages in block 1546. As an illustrative example, the computer system can compute the moving average of 3 probabilities determined by the model for 5 minute intervals/blocks of time. Computing the moving average can provide a smooth line and cleaned data for making decisions described below.

In block 1548, the computer system can determine whether a threshold quantity of consecutive received data signals satisfy one or more threshold phase criteria based on performing the moving average. The threshold quantity of consecutive received data signals can be defined by a decision window size, such as a period of time. As an illustrative example, the decision window size can be 30 minute windows. In some implementations, the decision window size can include 6 consecutive signals over 30 minute windows. The one or more threshold phase criteria can correspond to a defined probability or other numeric value indicating a likelihood that the items are experiencing defined phase (solid phase, liquid phase, mixed phase). As an illustrative example, the numeric value can include 0.7. The decision in block 1548 can then be whether 6 consecutive data signals during a 30 minute window of time had corresponding model output values above 0.7, or 70%, indicating that the items are likely solid. If at least 6 consecutive data signals during the 30 minute window had model output values above 0.7, then the items can be identified as being in a solid state. The numeric value can be defined differently based on parameters, such as the type of the items, the packaging, the materials, etc. In some implementations, the decision in block 1548 can be based on comparing the consecutive data signals in a predetermined window of time to multiple threshold values or ranges (e.g., a threshold value/range for solid phase probability, a threshold value/range for liquid phase probability, a threshold value/range for mixed phase probability).

If the one or more threshold phase criteria is not satisfied in block 1548, then the computer system can return to block 1502, receive the data signals, and continue processing the data signals to detect phase information for the cases/pallets of items. The computer system can continue performing the process 1500 until the phase and/or temperature of the items can be determined. The computer system may continue performing the process 1500 until user input is received indicating that the process 1500 can be stopped (e.g., the case/pallet of items is removed from a controlled environment, the sensor device is removed from the case/pallet and/or affixed to another case/pallet).

If the one or more threshold phase criteria is satisfied in block 1548, then the computer system can proceed to block 1550, in which the computer system can identify the items in the case/pallet as having the mixed, solid, or liquid phase that corresponds to the threshold quantity of the consecutive received data. In the illustrative example above, if 6 consecutive data points during a 30 minute window of time have respective model output probabilities of 0.7 for the solid phase, then the computer system can determine that the one or more threshold phase criteria is satisfied and the items in the case/pallet are likely in the solid phase state.

The computer system can then return information about the phase of the items in the case/pallet in block 1552. The information can include the phase determination made in block 1550. The information can include other information described herein, such as an internal temperature of the items, an expected amount of time until the items reach the next phase, a percentage of the items that are frozen, etc. Returning the information can include storing the information in a data store. Returning the information can include providing the information to computing devices of relevant users. The computing devices can present the information in one or more GUI displays. The users can view and interact with the presented information to perform actions and/or make decisions with regards to the case/pallet of items. In some implementations, returning the information can include transmitting the information to other computing systems, such as a control system, to perform operations that control and optimize usage of facility components (e.g., blast cells, refrigeration systems, cooling units, compressors, fans).

FIG. 16 is a flowchart of a process 1600 for training a phase model based on ultrasonic wave signals and temperature signals. The process 1600 can be performed by components described herein, such as the computer system 1320. In some implementations, the process 1600 can be performed by the control system 116 or any other computing systems/devices described. For illustrative purposes, the process 1600 is described from the perspective of a computer system.

Referring to the process 1600, the computer system can receive data signals captured for a case or pallet of items in block 1602. For example, the computer system can receive ultrasonic wave signals in block 1604. The computer system can receive temperature signals in block 1606. The signals can be received for one or more periods of time (e.g., current, historic). The signals can be generated using any of the sensor devices described herein. In some implementations, the signals for training can be generated by sensors that are positioned inside the case or pallet of items. The signals may additionally or alternatively be generated and collected for a variety of cases and/or pallets of items, including different types of items, items having different types of packaging, items having different types of materials, etc. As a result, the model can be trained and used to detect phase states of various different items having different parameters/conditions in a controlled environment, freight vehicle, facility, etc.

The computer system can encode and/or label the temperature data with corresponding phases in block 1608. In other words, the computer system may label the temperature data to identify what temperature values are indicative of different phases (e.g., liquid, solid, mixed) for the items. In some implementations, labels may correspond to different temperature bins, which can be used to provide an estimation of core temperature of a product instead of (or in addition to) a phase. With temperature, increased resolution may be achieved. The temperature bin labels can therefore be used for training a regression model, where an exact (or near exact) temperature can be predicted instead of a range in temperature The encoding/labeling can be performed automatically by the computer system. The encoding/labeling can additionally or alternatively be performed by a relevant user.

In block 1610, the computer system can correlate the ultrasonic wave signals with the encoded/labeled temperature data to generate correlated training data. In some implementations, the computer system may augment the correlated training data, by applying transformations, to increase diversity and improve the model's generalization and robustness. The computer system may additionally or alternatively perform normalization and/or dropout techniques to further improve robustness of the model before training the model.

The computer system can train a phase model to determine likelihoods of phases based on the correlated training data (block 1612). The phases can include, but are not limited to, a solid phase, a liquid phase, and a mixed phase. Sometimes, the model can be trained to determine a likelihood for less than all the phases (e.g., just the mixed phase). As described herein, the model can be a NN and/or CNN. NN and/or CNN training techniques may be used to train the model to determine the likelihoods of the phases. The model can be trained using techniques including but not limited to forward and backward passes. One or more optimizers may also be used in addition to or instead of a mini-batch gradient descent. For a loss function during training, categorical cross-entropy techniques may also be used.

In block 1614, the computer system perform model validation and/or testing techniques. For example, the computer system may adjust one or more parameters and/or hyper parameters based on the validation and/or testing. The computer system can iteratively improve the model accuracy. The computer system can perform block 1614 at predetermined time intervals (e.g., every 4 hours, every 6 hours, every 12 hours, once a day, every 1 day, every 3 days, every 5 days).

The computer system can return the trained phase model in block 1618. Returning the trained model can include storing the model in a data store. Returning the model can include deploying the model for runtime use at the computer system or at other computing systems described herein.

FIG. 17 is a flowchart of a process 1700 for determining a phase state of items using one or more trained machine learning models. In the process 1700, various inputs and machine learning models can be used to determine the phase state (e.g., solid, liquid, mixed) of the items in cases and/or pallets in a facility, such as a cold storage warehouse. The process 1700 can be performed by components described herein, such as the computer system 1320. In some implementations, the process 1700 can be performed by the control system 116 or any other computing systems/devices described. For illustrative purposes, the process 1700 is described from the perspective of a computer system.

Referring to the process 1700 in FIG. 17, the computer system can retrieve ultrasonic phase model outputs for a case or pallet of items in block 1702. In other words, the computer system may perform the process 1500 described in FIGS. 15A, 15B, and 15C to process ultrasonic wave signals from a sensor device (refer to the sensor device 1302 in FIG. 13A and the sensor device 1305 in FIG. 13B) and generate a phase state probability by providing the ultrasonic wave signals as input to the ultrasonic phase model described in the process 1500.

The computer system can also retrieve temperature phase model outputs for the case or pallet of items in block 1704. For example, the computer system can perform the process 800 described in FIGS. 8A and 8B to process temperature signals from a sensor device (refer to the sensor device 106 described herein) and generate information indicating internal temperature and/or phase state of the items by providing the temperature signals as input to the temperature phase model (or other models described in at least the process 800).

Blocks 1702 and 1704 can be performed in any order and/or at the same time. In some implementations, as described herein, the temperature signals and the ultrasonic wave signals can be processed together by the same model in the process 1700.

In block 1706, the computer system can retrieve phase state rules. For example, the computer system can retrieve one or more phase determination criteria in block 1708. As another example, the computer system can retrieve one or more trained phase determination models in block 1710. The phase state rules retrieved in blocks 1706-1710 can be used by the computer system to determine how to combine the model outputs from blocks 1702 and 1704 to make a determination of the phase state for the case/pallet of items. Moreover, the model(s) in block 1710 can be trained to correlate the model outputs from blocks 1702 and 1704 to determine the phase state for the case/pallet of items.

The model(s) can be trained similarly to the process 1600 described in FIG. 16 with regards to correlating ultrasonic signals with temperature signals to identify phase state information. For example, as described herein, the model(s) can be trained using forward and backward passes. The forward passes can be used to determine predictions and the backward passes can be used to determine gradients, which can further be used to update the model parameters to minimize loss and improve overall model performance. The forward and backward passes can be iterated over multiple epochs (passes through an entire training dataset), until the model's performance reaches a predetermined performance level and/or until a predetermined stop criteria has been met. An optimizer can be used for further training, such as an Adaptive Moment Estimation (ADAM). In some implementations, the training may also include applying a mini-batch gradient descent. Mini-batch gradient descent can be used to divide the training dataset into smaller subsets and computing gradient by averaging gradients of a loss function calculated on each mini-batch. The mini-batches can be randomly sampled from the training dataset. For the loss function, categorical cross-entropy may be used, especially since the model(s) is being trained for classification tasks.

The computer system can apply the phase state rules (e.g., the one or more phase determination criteria and/or the one or more trained phase determination models) to the ultrasonic phase model outputs and the temperature phase model outputs in block 1712.

Based on applying the phase state rules, the computer system can determine a phase of the case or pallet of items (block 1714).

The computer system can then return information based on the determined phase in block 1716. Refer to at least the process 1500 in FIGS. 15A, 15B, and 15C for further discussion about the returned information.

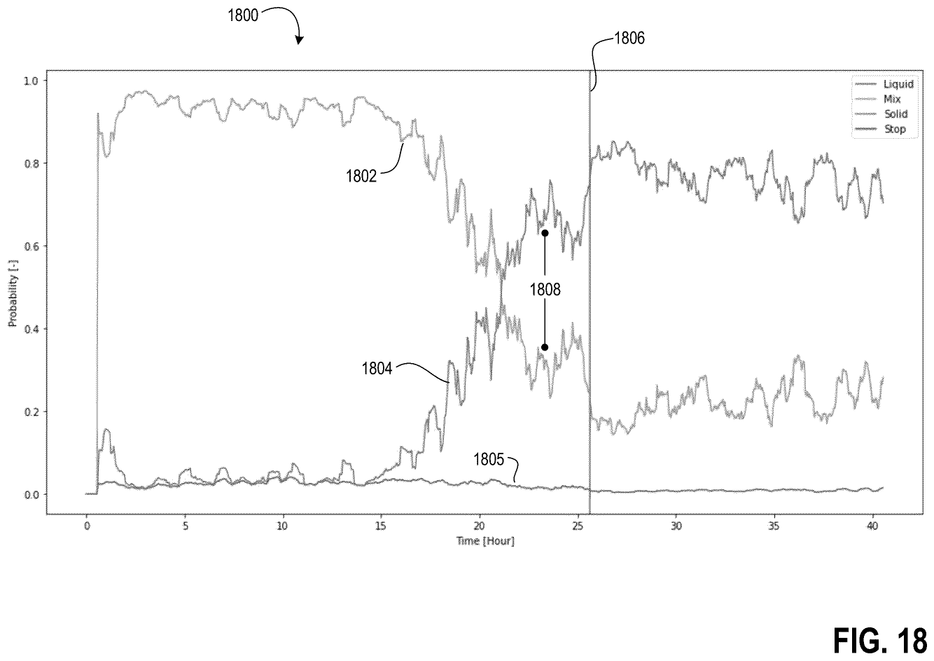

FIG. 18 is an example graph 1800 depicting a phase change detection using the disclosed technology. The graph 1800 depicts probability of phase states over time, the probability of phase states having been determined using the disclosed techniques, such as an ultrasonic phase model as described in reference to the process 1500 in FIGS. 15A, 15B, and 15C. As described herein, the model can determine probabilities for multiple phase states, such as a solid phase, a liquid phase, and a mixed phase, for a particular case of items or pallet of items (refer to blocks 1532-1538 in the process 1500).

Here, the graph 1800 includes a line 1802 depicting the probability of the mixed phase, a line 1804 that depicts the probability of the solid phase, and a line 1805 that depicts the probability of the liquid phase for the case/pallet of items. The lines 1802, 1804, and 1805 can be plotted using the techniques described herein, such as by performing a moving average of received data signals in blocks 1540-1546 of the process 1500.

For the first 20 or so hours, the mixed phase (line 1802) appears with high certainty, indicating that the model is most likely certain that the items are in the mixed phase (solid and liquid). The line 1805 depicting the probability of the liquid phase remains relatively constant and low for the duration of the modeling time (e.g., 0 to 40 hours). The line 1804 depicting the probability of the solid phase begins to increase between hours 15 through 25 while the line 1802 begins to decrease during the same time period, indicating that the items are becoming more solid with time. This transition of phases aligns with a cooling cycle that the items may be undergoing (e.g., in a blast cell or other controlled environment).

The model and/or the computer system(s) described herein can make a determination of what phase the items are experiencing at a time corresponding to vertical line 1806 (approximately 26 hours in to a cooling cycle for the items). As described in block 1548 of the process 1500, the computer system(s) can determine whether a threshold quantity of consecutive received data signals satisfy one or more threshold phase criteria based on performing the moving average. As an illustrative example, the computer system can determine whether, during the time corresponding to a phase change 1808 (approximately hours 20 through 25), the line 1802 depicting the mixed phase remains within a threshold probability range of 0.2-0.4 and the line 1804 depicting the solid phase remains within a threshold probability range of 0.5-0.7. If such threshold probability ranges are satisfied during the phase change 1808 time period, the computer system(s) can determine that the items have transitioned from the mixed phase to the solid phase. As another example, using a rolling moving average as described in reference to the process 1500, the computer system(s) can determine whether the one or more threshold phase criteria is satisfied for predetermined intervals of time (e.g., 5 hour windows). Once the computer system(s) reaches the 5-hour window between hours 20 and 25, the computer system(s) may determine that the one or more threshold phase criteria is met and that the items are transitioning (or have transitioned) to the solid phase from the mixed phase.

The time corresponding to the vertical line 1806 can be predetermined, can be based on settings/operations regarding the cooling cycle, and/or can be determined and adjusted based on information about the items undergoing the cooling cycle (e.g., item type, packaging, materials, customer information). As described above and in reference to the process 1500, the computer system(s) can determine time intervals for which to check the phase state of the items and the time corresponding to the vertical line 1806 can be identified at the time when the computer system(s) identifies that the items in fact changed phase states. As shown in FIG. 18, the vertical line 1806 is set after the end of the phase change 1808, and allows for accurately determining, by the computer system described herein, that the items have transitioned properly from the mixed phase (shown by the line 1802) to the solid phase (shown by the line 1804) during the cooling cycle.

While this specification contains many specific implementation details, these should not be construed as limitations on the scope of the disclosed technology or of what may be claimed, but rather as descriptions of features that may be specific to particular embodiments of particular disclosed technologies. Certain features that are described in this specification in the context of separate embodiments can also be implemented in combination in a single embodiment in part or in whole. Conversely, various features that are described in the context of a single embodiment can also be implemented in multiple embodiments separately or in any suitable subcombination. Moreover, although features may be described herein as acting in certain combinations and/or initially claimed as such, one or more features from a claimed combination can in some cases be excised from the combination, and the claimed combination may be directed to a subcombination or variation of a subcombination. Similarly, while operations may be described in a particular order, this should not be understood as requiring that such operations be performed in the particular order or in sequential order, or that all operations be performed, to achieve desirable results. Particular embodiments of the subject matter have been described. Other embodiments are within the scope of the following claims.

What is claimed is:

1. An apparatus for detecting phase state conditions of an object in a facility, the apparatus comprising:
a case;
a circuit board housed within the case;
a plurality of sensors communicably connected to the circuit board, wherein the plurality of sensors is configured to detect environmental conditions related to an object and generate signals based on the detected environmental conditions, wherein the plurality of sensors include two or more of temperature sensors, ultrasonic transducers, acoustic sensors, or humidity sensors;
a wireless transceiver inside the case and connected to the circuit board, wherein the wireless transceiver is configured to transmit the signals from the plurality of sensors to a computer system; and
a power source inside the case and configured to provide power to the plurality of sensors, the wireless transceiver, and the circuit board.

2. The apparatus of claim 1, further comprising a plurality of wires connected to the circuit board and at least partially extending from the case, wherein the plurality of sensors are attached to endpoints of the plurality of wires and configured to extend to different portions of the object so that the plurality of sensors detect the environmental conditions around the different portions of the object.

3. The apparatus of claim 1, wherein the case is configured to be removably affixed to a side of the object.

4. The apparatus of claim 1, wherein the apparatus is configured to be positioned amongst a plurality of objects on a pallet in a controlled environment, wherein the controlled environment comprises at least one of a cold storage room, a blast cell, or a temperature-controlled transit vehicle.

5. A system for detecting phase state conditions of an object in a facility, the system comprising:
a sensor device having a plurality of sensors that are configured to generate signals indicating detected conditions associated with an object, wherein the sensor device is configured to be removably affixed to the object; and
a computer system in network communication with the sensor device, wherein the computer system is configured to:
receive the signals generated by the plurality of sensors;
provide the received signals as inputs to one or more machine learning models;
receive, as output from the machine learning models, information identifying potential conditions of the object;
determining, based on applying one or more phase determination criteria to the information identifying the potential conditions of the object, a phase state of the object; and
returning information about the phase state of the object.

6. The system of claim 5, wherein the machine learning models were trained to generate values indicating likelihoods that the object is at one or more predetermined phases, the one or more predetermined phases including a solid phase, a liquid phase, and a mixed phase.

7. The system of claim 5, wherein the object comprises a case of items, the items comprising food products.

8. The system of claim 5, wherein the plurality of sensors comprise two or more of temperature sensors, acoustic sensors, ultrasonic transducers, or humidity sensors.

9. The system of claim 5, wherein the plurality of sensors include acoustic sensors, the acoustic sensors comprising at least one transducer configured to emit and receive an acoustic signal.

10. The system of claim 5, wherein the plurality of sensors comprise a first transducer configured to emit a sensor signal and a second transducer configured to receive an echo or reflection of the emitted sensor signal.

11. The system of claim 5, wherein determining, based on applying one or more phase determination criteria to the information identifying the potential conditions of the object, a phase state of the object comprises:
identifying a time interval to compute a moving average of the received signals;
for each data point in the received signals for the identified time interval, computing an average of data points within the time interval centered at the data point;
comparing the received signals to the computed moving averages;
determining whether a threshold quantity of consecutive signals amongst the received signals satisfy the one or more phase determination criteria based on performing the comparison; and
identifying the object as having a phase that corresponds to a phase of the threshold quantity of the consecutive signals amongst the received signals that satisfy the one or more phase determination criteria.

12. The system of claim 5, wherein the one or more machine learning models was trained, by the computer system, in a process that comprises:
receiving ultrasonic wave signals and temperature signals for a plurality of objects;
encoding or labeling the temperature signals with corresponding phase information;
correlating the ultrasonic waves signals with the encoded temperature signals to generate correlated training data; and
training the machine learning models to determine likelihoods of phases based on the correlated training data.

13. The system of claim 5, wherein the sensor device is configured to be positioned amongst a plurality of objects on a pallet in the facility.

14. A method for detecting phase state conditions of an object in a facility, the method comprising:
receiving, from at least one sensor, environmental conditions detected in or around an object in a controlled environment;
retrieving, from a data store, a model that was trained with machine learning techniques to correlate the detected environmental conditions with internal conditions of the object;
applying the model to the detected environmental conditions;
determining, based on applying the model, the internal conditions of the object;
generating, based on applying one or more rules to the internal conditions of the object, cooling operations for the controlled environment; and
returning the cooling operations for execution.

15. The method of claim 14, wherein:
the object comprises a case of items, and
the model was trained using labeled or encoded training data to determine the internal conditions of a plurality of different types of items and a plurality of different packaging and materials of the case,
wherein the model comprises parameters that are adjustable based on a type of the items inside the case.

16. The method of claim 14, wherein the at least one sensor is configured to (i) emit a wave signal that passes through the case and (ii) receive an echo or reflection of the wave signal when the wave signal reflects off a surface of the case that is opposite a location of the at least one sensor, wherein the wave signal comprises at least one of a soundwave signal or an ultrasonic wave signal.

17. The method of claim 16, the method further comprising:
receiving the echo or reflection of the wave signal;
filtering the echo or reflection of the wave signal with a band-pass filter;
processing the filtered wave signal to determine a time of flight (TOF) for each reflection of the wave signal; and
determining the internal conditions of the items inside the case based on applying the model to the TOF for each reflection of the wave signal.

18. The method of claim 14, wherein determining, based on applying the model, the internal conditions of the object comprises determining a percentage of the object that is frozen.

19. The method of claim 14, wherein the model was trained using a process comprising:
receiving temperature data for items inside a plurality of cases of items, wherein the temperature data comprises ambient temperature conditions surrounding the plurality of cases of items and internal temperature conditions of the items inside the plurality of cases;
applying a physics model to the received temperature data to correlate the ambient temperature conditions with the internal temperature conditions of the items inside the plurality of cases; and
training the model to predict the internal temperature conditions of the items based on the correlated temperature data.

20. The method of claim 14, wherein generating the cooling operation comprises:
generating a recommended action to complete a cooling cycle for the object, based on a determination that the internal conditions satisfy one or more cooling criteria that correspond to the object.
